(19) United States
(12) Patent Application Publication
(10) Pub. No.: US 2026/0233113 A1
(43) Pub. Date: Aug. 13, 2026

(54) Behavior Control System

(71) Applicant: SoftBank Group Corp., Tokyo (JP)

(72) Inventors: Masayoshi Son, Minato-ku, Tokyo (JP); Kenichi Yoshida, Minato-ku, Tokyo (JP); Satoshi Yano, Minato-ku, Tokyo (JP); Michio Ikeda, Minato-ku, Tokyo (JP)

(21) Appl. No.: 19/473,092

(22) PCT Filed: Apr. 11, 2024
(86) PCT No.: PCT/JP2024/014726

§ 371 Date: Oct. 6, 2025

(30) Foreign Application Priority Data
Apr. 11, 2023 (JP) 2023-064496
Apr. 13, 2023 (JP) 2023-065924
Apr. 13, 2023 (JP) 2023-065943
Apr. 26, 2023 (JP) 2023-072773
Apr. 27, 2023 (JP) 2023-073826
Apr. 28, 2023 (JP) 2023-075229
May 16, 2023 (JP) 2023-081014
May 19, 2023 (JP) 2023-083456
Jul. 24, 2023 (JP) 2023-120321

(51) Int. Cl. A63H 11/00; B25J 11/00

(52) U.S. Cl. CPC A63H 11/00; B25J 11/001; A63H 2200/00

(57) ABSTRACT

A behavior control system includes: an emotion determination unit that determines an emotion of a user or an emotion of a robot; and a behavior determination unit that generates a behavior content of the robot for a behavior of the user and the emotion of the user or the emotion of the robot based on a sentence generation model having a dialogue function that causes the user and the robot to have a dialogue with each other, and determines a behavior of the robot corresponding to the behavior content. The behavior determination unit generates, as the behavior content, an utterance content for a consultation from the user based on information regarding a specific person.

### TECHNICAL FIELD

The present disclosure relates to a behavior control system.

### BACKGROUND ART

Patent Literature 1 discloses a technology for determining an appropriate behavior of a robot for a state of a user. In the related art of Patent Literature 1, a reaction of the user in a case where the robot performs a specific behavior is recognized, and in a case where a behavior of the robot for the recognized reaction of the user cannot be determined, the behavior of the robot is updated by receiving information regarding a behavior appropriate for a recognized state of the user from a server.

Patent Literature 2 discloses a persona chatbot control method executed by at least one processor, the persona chatbot control method including: a step of receiving a user utterance; a step of adding the user utterance to a prompt including an instructional sentence associated with a description of a character of a chatbot; a step of encoding the prompt; and a step of inputting the encoded prompt into a language model to generate a chatbot utterance as a response to the user utterance.

### CITED LITERATURE

### Patent Literature

Patent Literature 1: Japanese Patent No. 6053847
Patent Literature 2: Japanese Patent Application Laid-Open No. 2022-180282

### SUMMARY OF INVENTION

### Technical Problem

However, in the related art, there is room for improvement in causing the robot to perform an appropriate behavior for a behavior of the user.

Further, in the related art, during a baseball game, it is easier for a batter to hit a ball in a case where the batter knows what pitch the pitcher is going to throw, but it is difficult to predict the next pitch.

### Solution to Problem

According to the first aspect of the disclosure, a behavior control system is provided. The behavior control system includes: an emotion determination unit that determines an emotion of a user or an emotion of a robot; and a behavior determination unit that generates a behavior content of the robot for a behavior of the user and the emotion of the user or the emotion of the robot based on a dialogue function of causing the user and the robot to have a dialogue with each other, and determines a behavior of the robot corresponding to the behavior content, in which the behavior determination unit generates, as the behavior content, an utterance content for a consultation from the user based on information regarding a specific person.

According to a second aspect of the disclosure, a behavior control system is provided. The behavior control system includes: an emotion determination unit that determines an emotion of a user or an emotion of a robot; and a behavior determination unit that generates a behavior content of the robot for a behavior of the user and the emotion of the user or the emotion of the robot based on a dialogue function that causes the user and the robot to have a dialogue with each other, and determines a behavior of the robot corresponding to the behavior content, in which in a case where it is determined that the user is a specific user including an individual who lives alone in isolation, the behavior determination unit switches to a specific mode in which the behavior of the robot is determined based on a communication count larger than a communication count in a normal mode in which the behavior is determined for a user other than the specific user. In a case where there is no dialogue with the specific user for a certain period of time in the specific mode, the behavior determination unit contacts a predetermined emergency contact.

According to a third aspect of the disclosure, a behavior control system is provided. The behavior control system includes: an emotion determination unit that determines an emotion of a user or an emotion of a robot; and a behavior determination unit that generates a behavior content of the robot for a behavior of the user and the emotion of the user or the emotion of the robot based on a dialogue function of causing the user and the robot to have a dialogue with each other, and determines a behavior of the robot corresponding to the behavior content, in which the robot is installed in a meeting room, and the behavior determination unit acquires a result of summarizing minutes of a past meeting held at the meeting room, and in a case where a statement whose content is similar to the summarized minutes has been made in a new meeting different from the past meeting, the behavior determination unit determines, as the behavior of the robot, outputting of advice information for the statement.

According to a fourth aspect of the disclosure, a behavior control system is provided. The behavior control system includes: a user state recognition unit that recognizes a user state including a behavior of a user; an emotion determination unit that determines an emotion of the user or an emotion of a robot; and a behavior determination unit that determines a behavior of the robot corresponding to the user state and the emotion of the user or the emotion of the robot based on a sentence generation model having a dialogue function of causing the user and the robot to have a dialogue with each other, in which the behavior determination unit generates a question corresponding to a concern of the user by using the sentence generation model, and determines, as the behavior of the robot, to make an utterance corresponding to the question.

Here, a robot includes a device that performs a physical operation, a device that outputs a video or a sound without performing a physical operation, and an agent that operates on software.

According to a fifth aspect of the disclosure, a behavior control system is provided. The behavior control system includes: a user state recognition unit that recognizes a user state including a behavior of a user; an emotion determination unit that determines an emotion of the user or an emotion of electronic equipment; and a behavior determination unit that determines a behavior of the electronic equipment corresponding to the user state and the emotion of the user or the emotion of the electronic equipment based on a sentence generation model having a dialogue function of causing the user and the electronic equipment to have a dialogue with each other, in which the behavior determination unit determines the behavior of the electronic equipment that supports health management of the user.

According to a sixth aspect of the disclosure, a behavior control system is provided. The behavior control system includes: a state recognition unit that recognizes a user state including a behavior of a user and a state of electronic equipment; an emotion determination unit that determines an emotion of the user or an emotion of the electronic equipment; and a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing, in which the equipment operation includes comforting the user, and in a case where the behavior determination unit determines, as the behavior of the electronic equipment, to comfort the user, the behavior determination unit determines an utterance content corresponding to the user state and the emotion of the user. The electronic equipment may be a robot, and the robot includes a device that performs a physical operation, a device that outputs a video or a sound without performing a physical operation, and an agent that operates on software.

According to a seventh aspect of the disclosure, a behavior control system is provided. The behavior control system includes: a state recognition unit that recognizes a user state including a behavior of a user and a state of electronic equipment; an emotion determination unit that determines an emotion of the user or an emotion of the electronic equipment; a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing; and a storage control unit that stores, in history data, event data including an emotion value determined by the emotion determination unit and data including the behavior of the user, in which the equipment operation includes provision of advice on health to the user, and in a case where the behavior determination unit determines, as the behavior of the electronic equipment, to provide the advice on health to the user, the behavior determination unit provides the advice on health to the user.

Here, a robot includes a device that performs a physical operation, a device that outputs a video or a sound without performing a physical operation, and an agent that operates on software.

According to an eighth aspect of the disclosure, a behavior control system is provided. The behavior control system includes: a state recognition unit that recognizes a user state including a behavior of a user and a state of electronic equipment; an emotion determination unit that determines an emotion of the user or an emotion of the electronic equipment; and a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing. The equipment operation includes provision of advice on a pregnant woman, and in a case where the behavior determination unit determines, as the behavior of the electronic equipment, to provide the advice on a pregnant woman, the behavior determination unit collects information regarding at least one of a pregnancy period and a post-partum period, and provides the advice on a pregnant woman based on the collected information.

Here, a robot includes a device that performs a physical operation, a device that outputs a video or a sound without performing a physical operation, and an agent that operates on software.

### BRIEF DESCRIPTION OF DRAWINGS

FIG. 1 schematically shows an example of a system 5 according to the present embodiment.

FIG. 2 schematically shows a functional configuration of a robot 100.

FIG. 3 schematically shows an example of an operation flow of the robot 100.

FIG. 4 schematically shows an example of a hardware configuration of a computer 1200.

FIG. 5 shows an emotion map 400 in which a plurality of emotions are mapped.

FIG. 6 shows an emotion map 900 in which a plurality of emotions are mapped.

FIG. 7(A) is an external view of a stuffed toy according to another embodiment, and FIG. 7(B) is an internal structural view of the stuffed toy.

FIG. 8 is a rear front view of the stuffed toy according to another embodiment.

FIG. 9A schematically shows a functional configuration of a robot 100 according to a second embodiment.

FIG. 9B schematically shows an example of an operation flow of collection processing performed by the robot 100 according to the second embodiment.

FIG. 9C schematically shows an example of an operation flow of autonomous processing performed by the robot 100 according to the second embodiment.

FIG. 9D schematically shows a functional configuration of a stuffed toy 100N according to a third embodiment.

FIG. 9E schematically shows a functional configuration of an agent system 2500 according to a fourth embodiment.

FIG. 9F shows an example of an operation of the agent system.

FIG. 9G shows an example of an operation of the agent system.

FIG. 9H schematically shows a functional configuration of smart glasses 2700 according to a seventh embodiment.

FIG. 9I shows an example of a usage aspect of an agent system in smart glasses.

FIG. 10A shows an outline of specific processing according to an eighth embodiment.

FIG. 10B schematically shows a functional configuration of a specific processing unit of a robot 100 according to the eighth embodiment.

FIG. 10C schematically shows an example of an operation flow of the specific processing performed by the robot 100 according to the eighth embodiment.

### DESCRIPTION OF EMBODIMENTS

Hereinafter, the present disclosure will be described through embodiments of the invention, but the following embodiments do not limit the invention according to the claims. In addition, not all combinations of features described in the embodiments are essential to the solution of the invention.

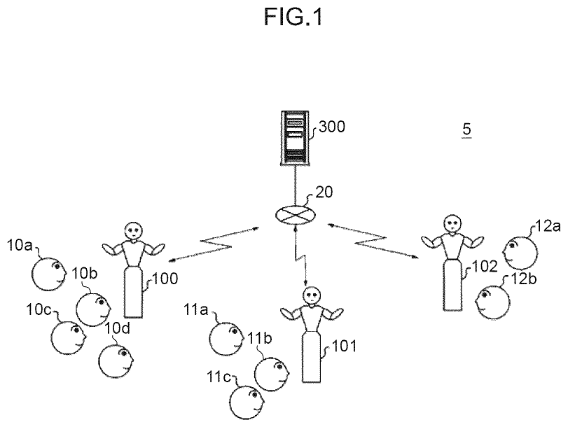

FIG. 1 schematically shows an example of a system 5 according to the present embodiment. The system 5 includes a robot 100, a robot 101, a robot 102, and a server 300. A user 10a, a user 10b, a user 10c, and a user 10d are users of the robot 100. A user 11a, a user 11b, and a user 11c are users of the robot 101. A user 12a and a user 12b are users of the robot 102. In the description of the present embodiment, the user 10a, the user 10b, the user 10c, and the user 10d may be collectively referred to as the user 10. Further, the user 11a, the user 11b, and the user 11c may be collectively referred to as the user 11. Further, the user 12a and the user 12b may be collectively referred to as the user 12. The robot 101 and the robot 102 have substantially the same functions as that of the robot 100. Therefore, the system 5 will be described focusing on the function of the robot 100.

The robot 100 has a conversation with the user 10 and provides a video to the user 10. At this time, the robot 100 has a conversation with the user 10, provides a video to the user 10, and the like in cooperation with the server 300 and the like that can perform communication via a communication network 20. For example, the robot 100 not only learns an appropriate conversation by itself, but also performs learning to have a more appropriate conversation with the user 10 in cooperation with the server 300. Further, the robot 100 causes the server 300 to record captured video data and the like of the user 10, requests the server 300 to transmit the video data and the like if necessary, and provides the video data and the like to the user 10.

Further, the robot 100 has an emotion value representing a type of an emotion thereof. For example, the robot 100 has the emotion value representing an intensity of each of emotions “joy”, “anger”, “sorrow”, “pleasure”, “comfort”, “discomfort”, “relief”, “anxiety”, “sadness”, “excitement”, “worry”, “reassurance”, “sense of fulfillment”, “sense of emptiness”, and “neutral”. For example, in the case of having a conversation with the user 10 in a state in which the emotion value of excitement is large, the robot 100 utters a speech at a high speed. As described above, the robot 100 can express the emotion thereof by a behavior.

Further, the robot 100 may be configured to determine a behavior of the robot 100 corresponding to an emotion of the user 10 by matching a sentence generation model and an emotion engine using an artificial intelligence (AI). Specifically, the robot 100 may be configured to recognize a behavior of the user 10, determine the emotion of the user 10 for the behavior of the user, and determine the behavior of the robot 100 corresponding to the determined emotion.

More specifically, in a case where the behavior of the user 10 is recognized, the robot 100 automatically generates a content of a behavior to be performed by the robot 100 for the behavior of the user 10 using the preset sentence generation model. The sentence generation model may be interpreted as an algorithm and operation for text-based automatic dialogue processing. Since the sentence generation model is known as disclosed in, for example, Japanese Patent Application Laid-Open No. 2018-081444 and chatGPT (Internet search <URL: https://openai.com/blog/chatgpt>), a detailed description thereof is omitted. Such a sentence generation model is implemented by a large language model (LLM).

As described above, in the present embodiment, it is possible to reflect the emotions of the user 10 and the robot 100 and various types of linguistic information in the behavior of the robot 100 by combining the large language model and the emotion engine. That is, according to the present embodiment, a synergistic effect can be obtained by combining the sentence generation model and the emotion engine.

Further, the robot 100 has a function of recognizing the behavior of the user 10. The robot 100 recognizes the behavior of the user 10 by analyzing a face image of the user 10 acquired by a camera function and a speech of the user 10 acquired by a microphone function. The robot 100 determines a behavior to be performed by the robot 100 based on the recognized behavior of the user 10 or the like.

The robot 100 stores a rule setting a behavior to be performed by the robot 100 based on the emotion of the user 10, the emotion of the robot 100, and the behavior of the user 10, and performs various behaviors according to the rule.

Specifically, the robot 100 has a reaction rule for determining the behavior of the robot 100 based on the emotion of the user 10, the emotion of the robot 100, and the behavior of the user 10. In the reaction rule, for example, a behavior of “laughing” is set as the behavior of the robot 100 for a case where the behavior of the user 10 is “laughing”. Further, in the reaction rule, a behavior of “apologizing” is set as the behavior of the robot 100 for a case where the behavior of the user 10 is “getting angry”. Further, in the reaction rule, a behavior of “answering” is set as the behavior of the robot 100 for a case where the behavior of the user 10 is “asking a question”. In the reaction rule, a behavior of “calling out” is set as the behavior of the robot 100 for a case where the behavior of the user 10 is “being sad”.

In a case where the robot 100 recognizes that the behavior of the user 10 is “getting angry”, the robot 100 selects the behavior of “apologizing” set in the reaction rule as a behavior to be performed by the robot 100 based on the reaction rule. For example, in a case where the behavior of “apologizing” is selected, the robot 100 performs the behavior of “apologizing” and outputs a speech representing words of “apology”.

Further, in a case where a condition that the emotion of the robot 100 is “neutral” (that is, “joy”=0, “anger”=0, “sorrow”=0, and “pleasure”=0) and a state of the user 10 is “alone and looking lonely” is satisfied, a content of a change in the emotion of the robot 100 to “worried” is determined, and it is determined that the behavior of “calling out” can be performed.

In a case where the robot 100 recognizes that the current emotion of the robot 100 is “neutral” and the user 10 is alone and looks lonely, the emotion value of “sorrow” of the robot 100 is increased based on the reaction rule. Further, the robot 100 selects the behavior of “calling out” set in the reaction rule as a behavior to be performed for the user 10. For example, in a case where the behavior of “calling out” is selected, the robot 100 converts a phrase “What's wrong?” expressing that the robot 100 is worried into a sympathetic voice, and outputs the voice.

Further, the robot 100 transmits, to the server 300, user reaction information indicating that a positive reaction has been obtained from the user 10 for the behavior. Examples of the user reaction information include the user behavior of “getting angry”, the behavior of the robot 100 of “apologizing”, the positive reaction of the user 10, and an attribute of the user 10.

The server 300 stores the user reaction information received from the robot 100. The server 300 receives and stores the user reaction information not only from the robot 100 but also from each of the robot 101 and the robot 102. Then, the server 300 analyzes the user reaction information from the robot 100, the robot 101, and the robot 102, and updates the reaction rule.

The robot 100 receives the updated reaction rule from the server 300 by inquiring the server 300 about the updated reaction rule. The robot 100 incorporates the updated reaction rule into the reaction rule stored in the robot 100. As a result, the robot 100 can incorporate the reaction rule acquired by the robot 101, the robot 102, or the like into the reaction rule thereof.

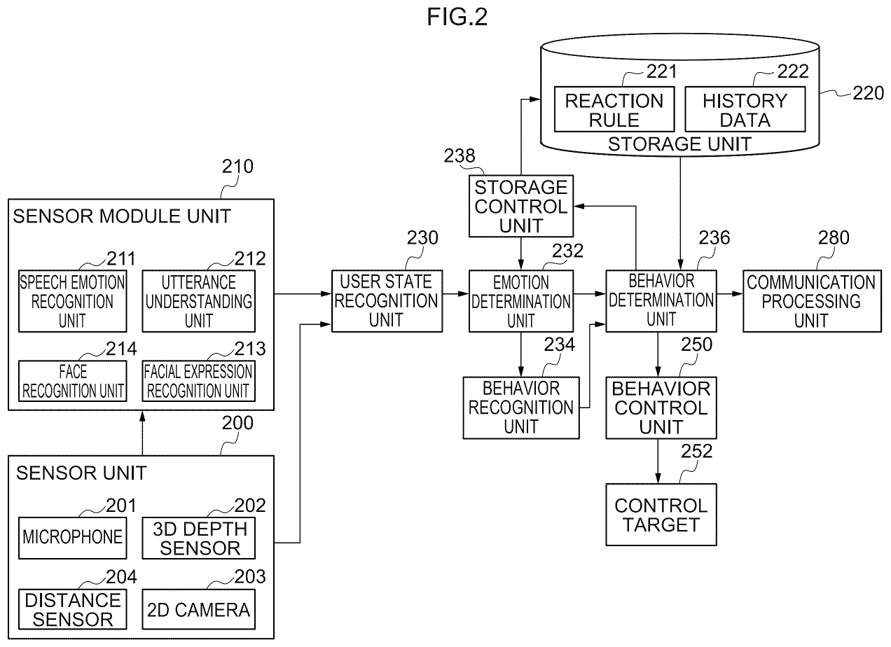

FIG. 2 schematically shows a functional configuration of the robot 100. The robot 100 includes a sensor unit 200, a sensor module unit 210, a storage unit 220, a user state recognition unit 230, an emotion determination unit 232, a behavior recognition unit 234, a behavior determination unit 236, a storage control unit 238, a behavior control unit 250, a control target 252, and a communication processing unit 280.

The control target 252 includes a display device, a speaker, a light emitting diode (LED) of an eye portion, motors that drive an arm, a hand, a foot, and the like, and the like. A posture and a gesture of the robot 100 are controlled by controlling the motors for the arm, the hand, the foot, and the like. Some emotions of the robot 100 can be expressed by controlling the motors. Furthermore, a facial expression of the robot 100 can be expressed by controlling a light emission state of the LED of the eye portion of the robot 100. The posture, the gesture and the facial expression of the robot 100 are examples of an attitude of the robot 100.

The sensor unit 200 includes a microphone 201, a 3D depth sensor 202, a 2D camera 203, and a distance sensor 204. The microphone 201 continuously detects a speech and outputs speech data. The microphone 201 may be provided at a head portion of the robot 100 and may have a function of performing binaural recording. The 3D depth sensor 202 detects an outline of an object by continuously radiating an infrared pattern and analyzing the infrared pattern based on an infrared image continuously captured by an infrared camera. The 2D camera 203 is an example of an image sensor. The 2D camera 203 performs imaging with visible light and generates video information of visible light. The distance sensor 204 detects a distance to an object by emitting, for example, a laser beam or an ultrasonic wave. The sensor unit 200 may further include a clock, a gyro sensor, a touch sensor, a sensor for motor feedback, and the like.

Among the components of the robot 100 shown in FIG. 2, the components other than the control target 252 and the sensor unit 200 are examples of components included in a behavior control system included in the robot 100. The behavior control system of the robot 100 controls the control target 252.

The storage unit 220 includes a reaction rule 221 and history data 222. The history data 222 includes a history of the past emotion value and behavior of the user 10. The history of the emotion value and the behavior is recorded for each user 10 by being associated with identification information of the user 10, for example. At least a part of the storage unit 220 is implemented by a storage medium such as a memory. A person DB that stores a face image of the user 10, attribute information of the user 10, and the like may be included. Among the components of the robot 100 shown in FIG. 2, functions of the components other than the control target 252, the sensor unit 200, and the storage unit 220 can be implemented by a CPU operating based on a program. For example, the functions of the components can be implemented as an operation of the CPU by basic software (operating system (OS)) and a program operating on the OS.

The sensor module unit 210 includes a speech emotion recognition unit 211, an utterance understanding unit 212, a facial expression recognition unit 213, and a face recognition unit 214. Information detected by the sensor unit 200 is input to the sensor module unit 210. The sensor module unit 210 analyzes the information detected by the sensor unit 200 and outputs an analysis result to the user state recognition unit 230.

The speech emotion recognition unit 211 of the sensor module unit 210 analyzes a speech of the user 10 detected by the microphone 201 to recognize the emotion of the user 10. For example, the speech emotion recognition unit 211 extracts a feature amount such as a frequency component of a speech and recognizes the emotion of the user 10 based on the extracted feature amount. The utterance understanding unit 212 analyzes the speech of the user 10 detected by the microphone 201 and outputs text information indicating an utterance content of the user 10.

The facial expression recognition unit 213 recognizes a facial expression of the user 10 and the emotion of the user 10 from an image of the user 10 captured by the 2D camera 203. For example, the facial expression recognition unit 213 recognizes the facial expression and the emotion of the user 10 based on shapes, positional relationships, and the like of the eyes and the mouth.

The face recognition unit 214 recognizes the face of the user 10. The face recognition unit 214 recognizes the user 10 by matching a face image stored in the person DB (not shown) with a face image of the user 10 captured by the 2D camera 203.

The user state recognition unit 230 recognizes a state of the user 10 based on the information analyzed by the sensor module unit 210. For example, processing mainly related to perception is performed using an analysis result of the sensor module unit 210. For example, perception information such as “Dad is alone” and “There is a 90% probability that dad is not smiling” is generated. Processing of understanding the meaning of the generated perception information is performed. For example, semantic information such as “Dad is alone and looks lonely” is generated.

The emotion determination unit 232 determines an emotion value indicating the emotion of the user 10 based on the information analyzed by the sensor module unit 210 and the state of the user 10 recognized by the user state recognition unit 230. For example, the emotion value indicating the emotion of the user 10 is acquired by inputting the information analyzed by the sensor module unit 210 and the recognized state of the user 10 to a neural network trained in advance.

Here, the emotion value indicating the emotion of the user 10 is a value indicating whether the emotion of the user is positive or negative. For example, the emotion value has a positive value in a case where the emotion of the user is a bright emotion accompanied by pleasure or a sense of calm, such as “joy”, “pleasure”, “comfort”, “relief”, “excitement”, “reassurance”, or “sense of fulfillment”, and the emotion value becomes larger as the emotion becomes brighter. The emotion value has a negative value in a case where the emotion of the user is an unpleasant emotion such as “anger”, “sorrow”, “discomfort”, “anxiety”, “sadness”, “worry”, or “sense of emptiness”, and the more unpleasant the emotion is, the larger the absolute value of the negative value becomes. In a case where the emotion of the user is not any of the above (“neutral”), the emotion value has a value of 0.

Further, the emotion determination unit 232 determines an emotion value indicating the emotion of the robot 100 based on the information analyzed by the sensor module unit 210 and the state of the user 10 recognized by the user state recognition unit 230.

The emotion value of the robot 100 includes an emotion value for each of a plurality of emotion classifications, and is, for example, a value (0 to 5) indicating an intensity of each of “joy”, “anger”, “sorrow”, and “pleasure”.

Specifically, the emotion determination unit 232 determines the emotion value indicating the emotion of the robot 100 according to a rule for updating the emotion value of the robot 100, the rule being set in association with the information analyzed by the sensor module unit 210 and the state of the user 10 recognized by the user state recognition unit 230.

For example, in a case where the user state recognition unit 230 recognizes that the user 10 looks lonely, the emotion determination unit 232 increases the emotion value of “sorrow” of the robot 100. Further, in a case where the user state recognition unit 230 recognizes that the user 10 is smiling, the emotion determination unit 232 increases the emotion value of “joy” of the robot 100.

The emotion determination unit 232 may determine the emotion value indicating the emotion of the robot 100 in further consideration of a state of the robot 100. For example, in a case where the remaining battery level of the robot 100 is low, a case where the surrounding environment of the robot 100 is dark, or the like, the emotion determination unit 232 may increase the emotion value of “sorrow” of the robot 100. Furthermore, in the case of the user 10 who continues to speak to the robot 100 despite the low remaining battery level, the emotion determination unit 232 may increase the emotion value of “anger”.

The behavior recognition unit 234 recognizes the behavior of the user 10 based on the information analyzed by the sensor module unit 210 and the state of the user 10 recognized by the user state recognition unit 230. For example, a probability of each of a plurality of predetermined behavior classifications (for example, “laughing”, “getting angry”, “asking a question”, and “being sad”) is acquired by inputting the information analyzed by the sensor module unit 210 and the recognized state of the user 10 to the neural network trained in advance, and a behavior classification having the highest probability is recognized as the behavior of the user 10.

As described above, in the present embodiment, the robot 100 acquires an utterance content of the user 10 after specifying the user 10, but in acquiring and using the utterance content, the behavior control system of the robot 100 according to the present embodiment considers protection of personal information and privacy of the user 10 in addition to acquisition of necessary consent according to laws and regulations from the user 10.

The behavior determination unit 236 determines a behavior corresponding to the behavior of the user 10 recognized by the behavior recognition unit 234, based on the current emotion value of the user 10 determined by the emotion determination unit 232, the history data 222 of the past emotion value determined by the emotion determination unit 232 before the current emotion value of the user 10 is determined, and the emotion value of the robot 100. In the present embodiment, a case where the behavior determination unit 236 uses one most recent emotion value included in the history data 222 as the past emotion value of the user 10 is described, but the disclosed technology is not limited to such an aspect. For example, the behavior determination unit 236 may use a plurality of most recent emotion values as the past emotion values of the user 10, or may use emotion values from a unit period earlier, such as one day ago, as the past emotion values of the user 10. Further, the behavior determination unit 236 may determine the behavior corresponding to the behavior of the user 10 in further consideration of the history of the past emotion value of the robot 100 in addition to the current emotion value of the robot 100. The behavior determined by the behavior determination unit 236 includes the gesture made by the robot 100 or an utterance content of the robot 100.

The behavior determination unit 236 according to the present embodiment determines, as the behavior corresponding to the behavior of the user 10, the behavior of the robot 100 based on a combination of the past emotion value and the current emotion value of the user 10, the emotion value of the robot 100, the behavior of the user 10, and the reaction rule 221. For example, in a case where the past emotion value of the user 10 is a positive value and the current emotion value is a negative value, the behavior determination unit 236 determines a behavior for positively changing the emotion value of the user 10 as the behavior corresponding to the behavior of the user 10.

In the reaction rule 221, the behavior of the robot 100 based on a combination of the past emotion value and the current emotion value of the user 10, the emotion value of the robot 100, and the behavior of the user 10 is set. For example, a combination of a gesture and an utterance content when encouraging the user 10 with a gesture is set as the behavior of the robot 100 in a case where the past emotion value of the user 10 is a positive value, the current emotion value is a negative value, and the behavior of the user 10 is being sad.

For example, in the reaction rule 221, behaviors of the robot 100 are set for all combinations of patterns of the emotion value of the robot 100 (1296 patterns which correspond to the fourth power of six values of “0” to “5” of “joy”, “anger”, “sorrow”, and “pleasure”), patterns of a combination of the past emotion value and the current emotion value of the user 10, and a behavior pattern of the user 10. That is, for each pattern of the emotion value of the robot 100, the behavior of the robot 100 based on the behavior pattern of the user 10 is determined for each of a plurality of combinations of the past emotion value and the current emotion value of the user 10, such as a combination of a negative value and a negative value, a combination of a negative value and a positive value, a combination of a positive value and a negative value, a combination of a positive value and a positive value, a combination of a negative value and a value indicating the neutral emotion, and a combination of a value indicating the neutral emotion and a value indicating the neutral emotion. The behavior determination unit 236 may transition to an operation mode of determining the behavior of the robot 100 by using the history data 222, for example, in a case where the user 10 has made an utterance that intends to continue a conversation of the past topic, such as “I want to talk about the topic we discussed earlier”.

In the reaction rule 221, at least one of a gesture and a statement content may be set as the behavior of the robot 100 for each pattern (1296 patterns) of the emotion value of the robot 100, with at most one behavior per pattern. Alternatively, in the reaction rule 221, at least one of the gesture and the statement content may be set as the behavior of the robot 100 for each group of the patterns of the emotion values of the robot 100.

An intensity of each gesture included in the behavior of the robot 100 and set in the reaction rule 221 is set in advance. An intensity of each utterance content included in the behavior of the robot 100 and set in the reaction rule 221 is set in advance.

The storage control unit 238 determines whether or not to store data including the behavior of the user 10 in the history data 222 based on a predetermined behavior intensity for the behavior determined by the behavior determination unit 236 and the emotion value of the robot 100 determined by the emotion determination unit 232.

Specifically, in a case where the total sum of the emotion values of the plurality of emotion classifications of the robot 100 and a total intensity value, which is the sum of the predetermined intensity for the gesture included in the behavior determined by the behavior determination unit 236 and the predetermined intensity for the utterance content included in the behavior determined by the behavior determination unit 236, are equal to or larger than thresholds, the storage control unit 238 determines to store the data including the behavior of the user 10 in the history data 222.

In a case where the storage control unit 238 determines to store the data including the behavior of the user 10 in the history data 222, the behavior determined by the behavior determination unit 236, the information (for example, any surrounding information such as data such as a sound, an image, and a scent at that time) analyzed by the sensor module unit 210 over a certain period prior to the current time point, and the state (for example, the facial expression or emotion of the user 10) of the user 10 recognized by the user state recognition unit 230 are stored in the history data 222.

The behavior control unit 250 controls the control target 252 based on the behavior determined by the behavior determination unit 236. For example, in a case where the behavior determination unit 236 determines a behavior including an utterance, the behavior control unit 250 causes the speaker included in the control target 252 to output a speech. At this time, the behavior control unit 250 may determine an utterance speed of the speech based on the emotion value of the robot 100. For example, the behavior control unit 250 determines a higher utterance speed as the emotion value of the robot 100 is larger. In this manner, the behavior control unit 250 determines an execution mode of the behavior determined by the behavior determination unit 236 based on the emotion value determined by the emotion determination unit 232.

The behavior control unit 250 may recognize a change in the emotion of the user 10 for execution of the behavior determined by the behavior determination unit 236. For example, the change in the emotion may be recognized based on the speech or facial expression of the user 10. In addition, the change in the emotion of the user 10 may be recognized based on detection of an impact applied to the touch sensor included in the sensor unit 200. In a case where an impact is detected by the touch sensor included in the sensor unit 200, it may be recognized that the emotion of the user 10 has become worse, and in a case where it is determined that the reaction of the user 10 is smiling or being happy based on a detection result of the touch sensor included in the sensor unit 200, it may be recognized that the emotion of the user 10 has been improved. Information indicating the reaction of the user 10 is output to the communication processing unit 280.

Further, after the behavior control unit 250 performs the behavior determined by the behavior determination unit 236 in the execution mode determined according to the emotion of the robot 100, the emotion determination unit 232 further changes the emotion value of the robot 100 based on the reaction of the user for the execution of the behavior. Specifically, the emotion determination unit 232 increases the emotion value of “joy” of the robot 100 in a case where the reaction of the user for the behavior determined by the behavior determination unit 236 and performed for the user in the execution form determined by the behavior control unit 250 is not negative, and the emotion determination unit 232 increases the emotion value of “sorrow” of the robot 100 in a case where the reaction of the user for the behavior determined by the behavior determination unit 236 and performed for the user in the execution form determined by the behavior control unit 250 is negative.

Furthermore, the behavior control unit 250 expresses the emotion of the robot 100 based on the determined emotion value of the robot 100. For example, in a case where the emotion value of “joy” of the robot 100 is increased, the behavior control unit 250 controls the control target 252 to cause the robot 100 to make a joyful gesture. Further, in a case where the emotion value of “sorrow” of the robot 100 is increased, the behavior control unit 250 controls the control target 252 such that the posture of the robot 100 becomes a drooping posture.

The communication processing unit 280 is responsible for communication with the server 300. As described above, the communication processing unit 280 transmits the user reaction information to the server 300. Further, the communication processing unit 280 receives the updated reaction rule from the server 300. In a case where the updated reaction rule is received from the server 300, the communication processing unit 280 updates the reaction rule 221.

The server 300 performs communication between the server 300 and the robot 100, the robot 101, and the robot 102, receives the user reaction information transmitted from the robot 100, and updates the reaction rule based on a reaction rule including a behavior for which a positive reaction has been obtained.

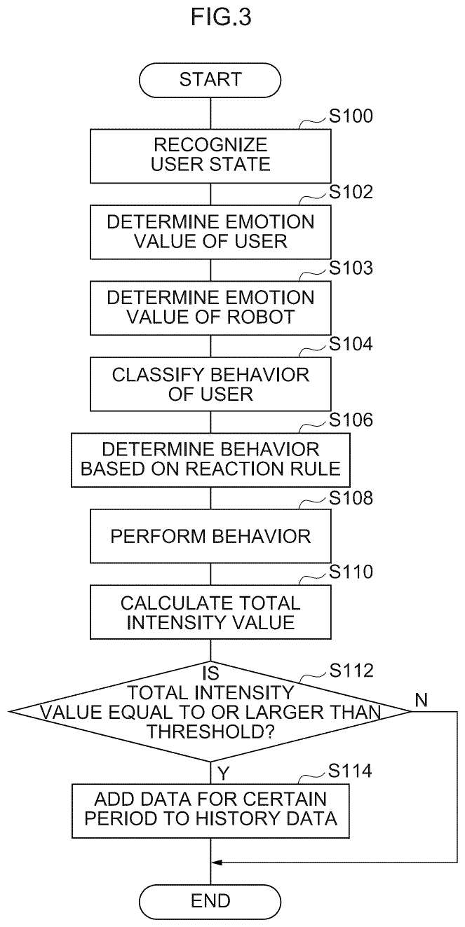

FIG. 3 schematically shows an example of an operation flow related to an operation of determining a behavior in the robot 100. The operation flow shown in FIG. 3 is repeatedly performed. At this time, it is assumed that the information analyzed by the sensor module unit 210 is input. “S” in the operation flow represents a step to be performed.

First, in step S100, the user state recognition unit 230 recognizes the state of the user 10 based on the information analyzed by the sensor module unit 210.

In step S102, the emotion determination unit 232 determines the emotion value indicating the emotion of the user 10 based on the information analyzed by the sensor module unit 210 and the state of the user 10 recognized by the user state recognition unit 230.

In step S103, the emotion determination unit 232 determines the emotion value indicating the emotion of the robot 100 based on the information analyzed by the sensor module unit 210 and the state of the user 10 recognized by the user state recognition unit 230. The emotion determination unit 232 adds the determined emotion value of the user 10 to the history data 222.

In step S104, the behavior recognition unit 234 recognizes a behavior classification of the user 10 based on the information analyzed by the sensor module unit 210 and the state of the user 10 recognized by the user state recognition unit 230.

In step S106, the behavior determination unit 236 determines the behavior of the robot 100 based on the combination of the current emotion value of the user 10 determined in step S102 and the past emotion value included in the history data 222, the emotion value of the robot 100, the behavior of the user 10 recognized by the behavior recognition unit 234, and the reaction rule 221.

In step S108, the behavior control unit 250 controls the control target 252 based on the behavior determined by the behavior determination unit 236.

In step S110, the storage control unit 238 calculates the total intensity value based on the predetermined behavior intensity for the behavior determined by the behavior determination unit 236 and the emotion value of the robot 100 determined by the emotion determination unit 232.

In step S112, the storage control unit 238 determines whether or not the total intensity value is equal to or larger than the threshold. In a case where the total intensity value is smaller than the threshold, the data including the behavior of the user 10 is not stored in the history data 222, and the processing ends. On the other hand, in a case where the total intensity value is equal to or larger than the threshold, the processing proceeds to step S114.

In step S114, the behavior determined by the behavior determination unit 236, the information analyzed by the sensor module unit 210 over a certain period prior to the current time point, and the state of the user 10 recognized by the user state recognition unit 230 are stored in the history data 222.

As described above, with the robot 100, the emotion value indicating the emotion of the robot 100 is determined based on the state of the user, and whether or not to store the data including the behavior of the user 10 in the history data 222 is determined based on the emotion value of the robot 100. As a result, a volume of the history data 222 that stores the data including the behavior of the user 10 can be reduced. Then, for example, in a case where the robot 100 determines that the state of the user after ten years matches the state of the user from ten years earlier, the robot 100 can read the history data 222 from ten years ago to present, to the user 10, the state of the user 10 from ten years earlier (for example, the facial expression or emotion of the user 10), and further, any surrounding information such as data of a sound, an image, and a scent at that time.

Further, with the robot 100, it is possible to cause the robot 100 to perform an appropriate behavior for the behavior of the user 10. Hitherto, a behavior of the user has been classified to determine a behavior including a facial expression or appearance of the robot. On the other hand, the robot 100 determines the current emotion value of the user 10 and performs a behavior for the user 10 based on the past emotion value and the current emotion value. Therefore, for example, in a case where the user 10 who seemed fine yesterday is depressed today, the robot 100 can make an utterance such as “You seemed fine yesterday. What's wrong today?”. Further, the robot 100 can also make an utterance with a gesture. Further, for example, in a case where the user 10 who was depressed yesterday seems fine today, the robot 100 can make an utterance such as “You seemed down yesterday, but you look fine today!”. Further, for example, in a case where the user 10 who seemed fine yesterday looks better today than yesterday, the robot 100 can make an utterance such as “You look better today than yesterday. Did anything good happen since yesterday?”. Further, for example, the robot 100 can make an utterance such as “You've been in a really stable mood lately. That's great!” for the user 10 whose emotion value is 0 or more and whose emotion value fluctuation continuously remains within a certain range.

Further, for example, in a case where the robot 100 asks the user 10, “Did you finish the homework you mentioned yesterday?”, and the user 10 answers “Yeah, I did”, the robot 100 can make a positive utterance such as “Good job!” and make a positive gesture such as applause or thumbs-up. Furthermore, for example, in a case where the user 10 makes an utterance “The presentation I talked about the day before yesterday went well”, the robot 100 can make a positive utterance such as “Nice effort!” and also make the above affirmative gesture. As described above, the robot 100 performs a behavior based on a history of the state of the user 10, whereby it can be expected that the user 10 feels a sense of closeness toward the robot 100.

In the above embodiment, a case where the robot 100 recognizes the user 10 by using the face image of the user 10 has been described, but the disclosed technology is not limited to such an aspect. For example, the robot 100 may recognize the user 10 by using a voice uttered by the user 10, a mail address of the user 10, an ID of a social network service (SNS) of the user 10, an ID card in which a wireless IC tag is embedded and which is possessed by the user 10, or the like.

The robot 100 is an example of electronic equipment including the behavior control system. An application target of the behavior control system is not limited to the robot 100, and the behavior control system can be applied to various types of electronic equipment. Further, functions of a server 300 may be implemented by one or more computers. At least some functions of the server 300 may be implemented by a virtual machine. Further, at least some functions of the server 300 may be implemented on a cloud.

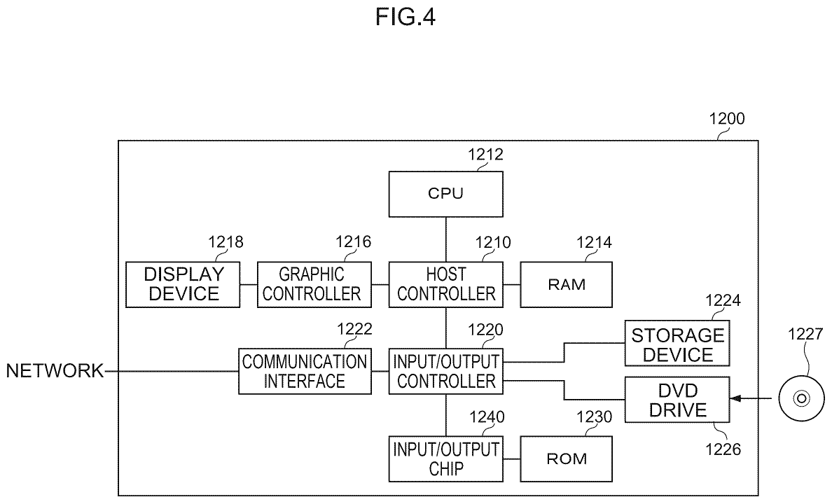

FIG. 4 schematically shows an example of a hardware configuration of a computer 1200 that functions as the robot 100 and the server 300. A program installed in the computer 1200 can cause the computer 1200 to function as one or more “units” of the device according to the present embodiment, or cause the computer 1200 to perform an operation associated with the device according to the embodiment or one or more “units” thereof, and/or can cause the computer 1200 to execute a process according to the embodiment or a stage of the process. Such a program may be executed by a CPU 1212 to cause the computer 1200 to perform a certain operation associated with some or all of the blocks in the flowcharts and block diagrams described herein.

The computer 1200 according to the embodiment includes the CPU 1212, a random access memory (RAM) 1214, and a graphics controller 1216, which are mutually connected by a host controller 1210. The computer 1200 also includes input/output units such as a communication interface 1222, a storage device 1224, a digital versatile disk (DVD) drive 1226, and an integrated circuit (IC) card drive, which are connected to the host controller 1210 via an input/output controller 1220. The DVD drive 1226 may be a DVD-ROM drive, a DVD-RAM drive, or the like. The storage device 1224 may be a hard disk drive, a solid state drive, or the like. The computer 1200 also includes a read only memory (ROM) 1230 and a legacy input/output unit such as a keyboard, which are connected to the input/output controller 1220 via an input/output chip 1240.

The CPU 1212 operates according to the program stored in the ROM 1230 and the RAM 1214, thereby controlling each unit. The graphics controller 1216 acquires image data generated by the CPU 1212 in a frame buffer or the like provided in the RAM 1214 or itself, and causes the image data to be displayed on a display device 1218.

The communication interface 1222 communicates with other electronic devices via a network. The storage device 1224 stores the program and data to be used by the CPU 1212 in the computer 1200. The DVD drive 1226 reads the program or data from a DVD-ROM 1227 or the like and provides the program or data to the storage device 1224. The IC card drive reads the program and data from an IC card and/or writes the program and data to the IC card.

The ROM 1230 stores therein a boot program to be executed by the computer 1200 at the time of activation and/or a program that depends on hardware of the computer 1200. The input/output chip 1240 may also connect various input/output units to the input/output controller 1220 via a USB port, a parallel port, a serial port, a keyboard port, a mouse port, or the like.

The program is provided by a computer-readable storage medium such as the DVD-ROM 1227 or the IC card. The program is read from the computer-readable storage medium, installed in the storage device 1224, the RAM 1214, or the ROM 1230, which is also an example of the computer-readable storage medium, and executed by the CPU 1212. Information processing described in these programs is read by the computer 1200 and provides cooperation between the programs and various types of hardware resources described above. The device or method may be configured by implementing operation or processing of information according to the use of the computer 1200.

For example, in a case where communication is performed between the computer 1200 and an external device, the CPU 1212 may execute a communication program loaded into the RAM 1214 and instruct the communication interface 1222 to execute communication processing based on processing described in the communication program. Under the control of the CPU 1212, the communication interface 1222 reads transmission data stored in a transmission buffer region provided in a recording medium such as the RAM 1214, the storage device 1224, the DVD-ROM 1227, or the IC card, transmits the read transmission data to the network, or writes reception data received from the network to a reception buffer region or the like provided on the recording medium.

In addition, the CPU 1212 may read a necessary part of or the entire file or database stored in an external recording medium such as the storage device 1224, the DVD drive 1226 (DVD-ROM 1227), the IC card, or the like into the RAM 1214, and may perform various types of processing on the data on the RAM 1214. Next, the CPU 1212 may write back the processed data to the external recording medium.

Various types of information such as various types of programs, data, tables, and databases may be stored in a recording medium and subjected to the information processing. The CPU 1212 may perform various types of processing on the data read from the RAM 1214, the various types of processing including various types of operations, the information processing, condition determination, conditional branching, unconditional branching, and information search/replacement, which are described throughout the disclosure and designated by a command sequence of a program, and write back the results to the RAM 1214. In addition, the CPU 1212 may search for information in a file, a database, or the like in the recording medium. For example, in a case where a plurality of entries each having an attribute value of a first attribute associated with an attribute value of a second attribute are stored in the recording medium, the CPU 1212 may search for an entry in which the attribute value of the first attribute satisfies a designated condition among the plurality of entries, read the attribute value of the second attribute stored in the entry, and thereby acquire the attribute value of the second attribute associated with the first attribute satisfying a predetermined condition.

The program or software module described above may be stored in a computer-readable storage medium on the computer 1200 or in the vicinity of the computer 1200. Further, a recording medium such as a hard disk or a RAM provided in a server system connected to a dedicated communication network or the Internet can be used as the computer-readable storage medium, thereby providing a program to the computer 1200 via the network.

The blocks in the flowcharts and block diagrams in the embodiment may represent stages of a process in which the operation is performed or “units” of the device that are responsible for performing the operation. Certain stages and “units” may be implemented by a dedicated circuit, a programmable circuit provided together with a computer-readable instruction stored on a computer-readable storage medium, and/or a processor provided together with the computer-readable instruction stored on the computer-readable storage medium. The dedicated circuit may include a digital and/or analog hardware circuit, and may include an integrated circuit (IC) and/or a discrete circuit. The programmable circuit may include a reconfigurable hardware circuit including, for example, AND, OR, XOR, NAND, NOR, and other logical operations, a flip-flop, a register, and a memory element, such as a field programmable gate array (FPGA) and a programmable logic array (PLA).

The computer-readable storage medium may include any tangible device capable of storing an instruction to be executed by a suitable device, so that the computer-readable storage medium having the instruction stored therein includes an article including an instruction that may be executed to create means for performing the operation specified in the flowcharts or block diagrams. Examples of the computer-readable storage medium may include an electronic storage medium, a magnetic storage medium, an optical storage medium, an electromagnetic storage medium, and a semiconductor storage medium. More specific examples of the computer-readable storage medium may include a floppy (registered trademark) disk, a diskette, a hard disk, a random access memory (RAM), a read only memory (ROM), an erasable programmable read only memory (EPROM or flash memory), an electrically erasable programmable read only memory (EEPROM), a static random access memory (SRAM), a compact disc read only memory (CD-ROM), a digital versatile disk (DVD), a Blu-Ray disk, a memory stick, and an integrated circuit card.

The computer-readable instruction may include a source code or an object code described in any combination of one or more programming languages, including an assembler instruction, an instruction-set-architecture (ISA) instruction, a machine instruction, a machine-dependent instruction, a microcode, a firmware instruction, state setting data, or an object-oriented programming language such as Smalltalk, JAVA (registered trademark), or C++, and a procedural programming language according to the related art, such as the “C” programming language or similar programming languages.

The computer-readable instruction may be provided for a processor of a general purpose computer, a special purpose computer, or another programmable data processing device, or a programmable circuit, either locally or via a local area network (LAN) or a wide area network (WAN) such as the Internet, to cause the processor of the general purpose computer, the special purpose computer, or the another programmable data processing device or the programmable circuit to execute the computer-readable instruction to generate means for performing the operation designated in the flowcharts or block diagrams. Examples of the processor include a computer processor, a processing unit, a microprocessor, a digital signal processor, a controller, and a microcontroller.

Although the disclosure has been described with reference to the embodiments, the technical scope of the disclosure is not limited to the scope described in the embodiments. It is apparent to those skilled in the art that various modifications or improvements can be made to the above embodiments. It is apparent from the description of the claims that such changed embodiments or improved embodiments can also be included in the technical scope of the disclosure.

It should be noted that an order of execution of processing such as operations, procedures, steps, and stages in the devices, systems, programs, and methods shown in the claims, the specification, and the drawings can be implemented in any order unless “before”, “prior to”, or the like is explicitly stated, and unless the output of the previous processing is used in the later processing. Even in a case where the operation flow in the claims, the specification, and the drawings is described using the terms “first”, “next”, and the like for convenience, it does not mean that it is essential to execute the operation flow in this order.

### Another Embodiment 1

The robot 100 according to the embodiment of the disclosure includes the emotion determination unit that determines the emotion of the user or the emotion of the robot, and the behavior determination unit that generates the behavior content of the robot for the behavior of the user and the emotion of the user or the emotion of the robot 100 based on a dialogue function that causes the user and the robot 100 to have a dialogue with each other, and determines the behavior of the robot 100 corresponding to the behavior content. The behavior determination unit 236 may generate, as the behavior content, an utterance content for a consultation from the user based on information regarding a specific person. Specifically, the robot 100 first learns the information regarding the specific person. The person may include a supervisor, a colleague, a junior, a relative, or the like of the user of the robot 100. The person is not limited thereto, and examples of the person may include an expert, a historical figure who does not currently exist, a famous person, and a person who is located in a distant place. Specifically, examples of the expert may include a diviner, a medium, a lawyer, a patent attorney, a judicial scrivener, a certified public accountant, a tax attorney, an administrative scrivener, a social insurance labor attorney, an architect, a real estate transaction agent, a financial planner (FP), and a loan consultant. Qualifications may be either national or private. The information regarding the specific person may include a voice, a speech habit, a thought pattern, an experience, and the like of the person.

The behavior determination unit 236 of the robot 100 that has learned the information regarding the specific person generates an utterance content for a content of the consultation from the user in a case where the consultation for a current individual specific problem of the user has been received from the user. Specifically, the behavior determination unit 236 generates a speech content corresponding to a content that the specific person could provide as an answer for the content of the consultation from the user, such as thoughts or advice of the person. More specifically, the speech content is generated by combining the sentence generation model that has learned the information regarding the specific person and the emotion engine.

In the case of generating the speech content corresponding to the content that the specific person could provide as the answer, the behavior determination unit 236 may reflect the voice or the speech habit of the specific person in the utterance content. Specifically, in a case where the supervisor, the manager, or the like of the user is selected as the specific person, the behavior determination unit 236 may generate the speech content corresponding to the content of the consultation from the user with a voice close to a voice of the supervisor or the like. Furthermore, in a case where a close friend, a sibling, or the like of the user is selected as the specific person, the behavior determination unit 236 may generate the speech content corresponding to the content of the consultation from the user with a voice close to a voice of the close friend, the sibling, or the like.

The behavior determination unit 236 may determine the gesture of the robot 100, which corresponds to the utterance content for the consultation from the user. The gesture may include a body gesture, a hand gesture, a facial expression, or the like that expresses an emotion, an intention, or the like or transmits the emotion, the intention, or the like to a counterpart. Specifically, in a case where the supervisor, the manager, or the like of the user is selected as the specific person, the behavior determination unit 236 may determine a motion such as crossing arms while speaking, making large arm movements while speaking, and averting a gaze from the user while speaking.

With the robot 100 of the disclosure, it is possible to provide, to the user, thoughts, actions, and advice of the specific person, including gestures imitating the voice, the speech habit, and the like of the person.

With the robot 100 of the disclosure, the user can immediately consult with a person who does not exist (such as a historical figure), a person who is located in a distant place, or the like. More specifically, even in a situation in which the user desires to seek brief advice from the supervisor, but the supervisor is working from home, away on a business trip, or otherwise absent from the office where the user is present, the user can find out, via the robot 100, what advice the supervisor would provide, without speaking directly with the supervisor.

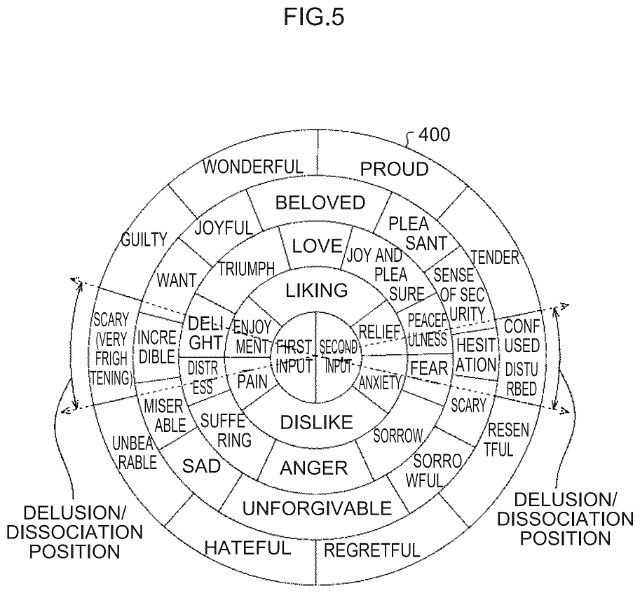

The emotion determination unit 232 may determine the emotion of the user according to a specific mapping. Specifically, the emotion determination unit 232 may determine the emotion of the user based on an emotion map (see FIG. 5) representing the specific mapping.

FIG. 5 is a diagram showing an emotion map 400 in which a plurality of emotions are mapped. In the emotion map 400, emotions are arranged radially in concentric circles from the center. The closer to the center of the concentric circle, the more primitive the emotion is. Emotions representing states and behaviors arising from a mental state are arranged on an outer side of the concentric circle. The emotion is a concept including emotional reactions and psychological conditions. Emotions arising from reactions generally occurring in the brain are arranged on a left side of the concentric circle. Emotions induced by situation determination are generally arranged on a right side of the concentric circle. Emotions arising from reactions generally occurring in the brain and induced by situation determination are arranged in an upward direction and a downward direction of the concentric circle. Further, emotions of “comfort” are arranged on an upper side of the concentric circle, and emotions of “discomfort” are arranged on a lower side of the concentric circle. As described above, in the emotion map 400, a plurality of emotions are mapped based on a structure in which emotions arise, and emotions that are likely to arise at the same time are mapped close to each other.

(1) For example, in a case where the emotion engine, which is the emotion determination unit 232 of the robot 100, detects an emotion about every 100 msec, determination of a reaction operation (for example, the backchannel response) of the robot 100 may be performed at at least a similar frequency to the detection frequency (100 msec) of the emotion engine, or may be performed at a frequency higher than the detection frequency. The detection frequency of the emotion engine may be interpreted as a sampling rate.

The emotion is detected about every 100 msec, and the reaction operation (for example, the backchannel response) is performed immediately in conjunction with the detection, whereby an unnatural backchannel response is not performed, and a natural and smooth dialogue can be implemented. The robot 100 performs the reaction operation (such as the backchannel response) according to a direction and a magnitude (intensity) in the mandala-like emotion map 400. The detection frequency (sampling rate) of the emotion engine is not limited to 100 ms, and may be changed according to a situation (such as a case of playing sports), an age of the user, or the like.

(2) According to the emotion map 400, a direction and an intensity of an emotion may be set in advance, and a backchannel response motion and an intensity of the backchannel response may be set. For example, in a case where the robot 100 feels a sense of stability, relief, or the like, the robot 100 continues to listen while nodding. In a case where the robot 100 feels anxious, lost, or suspicious, the robot 100 may tilt the head thereof or stop movement of the head.

Such emotions are distributed at 3 o'clock positions on the emotion map 400 and usually range between relief and anxiety. In the right half of the emotion map 400, since situational awareness takes precedence over internal sensations, a calm impression is conveyed.

(3) In a case where the robot 100 experiences pleasure from being praised, a filler such as “Oh” may be inserted before an utterance. In a case where the robot 100 feels a sense of pain from receiving harsh words, a filler “Ugh!” may be inserted before an utterance. Further, the robot 100 may also perform a physical reaction such as a gesture of crouching while saying “Ugh!”. Such emotions are distributed around 9 o'clock positions on the emotion map 400.

(4) In the left half of the emotion map 400, internal sensations (reactions) take precedence over situational awareness. Therefore, an impression of an involuntary reaction can be conveyed.

In a case where the robot 100 has a favorable impression through situational awareness while experiencing an internal sensation (reaction) of acceptance, the robot 100 may nod deeply while looking at the counterpart, or may utter “Mm-hmm”. In this manner, the robot 100 may produce a balanced favorable impression for the counterpart, that is, perform a behavior expressing permissiveness or tolerance toward the counterpart. Such emotions are distributed around 12 o'clock positions in the emotion map 400.

On the other hand, in a case where the robot 100 has an unfavorable impression through situational awareness while experiencing an internal sensation (reaction) of discomfort, the robot 100 may shake the head sideways, and in a case where the robot 100 feels hatred, the robot 100 may illuminate the LED of the eye in red and glare at the counterpart. Such emotions are distributed around 6 o'clock positions in the emotion map 400.

(5) Since an inner side of the emotion map 400 represents feelings and an outer side of the emotion map 400 represents behaviors, the emotions on the outer side of the emotion map 400 are more visible (appear in behaviors).

(6) In a case where the robot 100 listens to a speech of a person while feeling relief distributed around the 3 o'clock position on the emotion map 400, the robot 100 slightly nods the head vertically and says “Hmm-hmm”. However, in a case where the robot 100 feels love distributed around the 12 o'clock position, the robot 100 may perform a more forceful and deeper vertical nod.

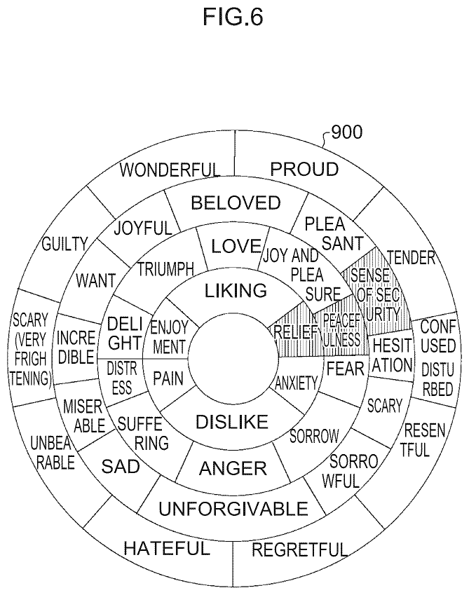

The emotion determination unit 232 inputs the information analyzed by the sensor module unit 210 and the recognized state of the user 10 to the neural network trained in advance, acquires the emotion value indicating each emotion indicated in the emotion map 400, and determines the emotion of the user 10. The neural network is trained in advance based on a plurality of pieces of learning data, which are a combination of the information analyzed by the sensor module unit 210, the recognized state of the user 10, and the emotion value indicating each emotion indicated in the emotion map 400. Furthermore, the neural network is trained such that emotions arranged close to each other as in an emotion map 900 shown in FIG. 6 have close values. FIG. 6 shows an example in which a plurality of emotions such as “relief”, “peacefulness”, and “sense of security” have similar emotion values.

Further, the emotion determination unit 232 may determine the emotion of the robot 100 according to the specific mapping. Specifically, the emotion determination unit 232 inputs the information analyzed by the sensor module unit 210, the state of the user 10 recognized by the user state recognition unit 230, and the state of the robot 100 to the neural network trained in advance, acquires the emotion value indicating each emotion indicated in the emotion map 400, and determines the emotion of the robot 100. The neural network is trained in advance based on a plurality of pieces of learning data, which are a combination of the information analyzed by the sensor module unit 210, the recognized state of the user 10, the state of the robot 100, and the emotion value indicating each emotion shown in the emotion map 400. For example, the neural network is trained based on the learning data indicating that the emotion value “3” of “joyful” is obtained in a case where it is recognized that the robot 100 is being stroked by the user 10 from an output of the touch sensor (not shown), and the learning data indicating that the emotion value “3” of “anger” is obtained in a case where it is recognized that the robot 100 is being hit by the user 10 from an output of an acceleration sensor (not shown). Furthermore, the neural network is trained such that emotions arranged close to each other as in an emotion map 900 shown in FIG. 6 have close values.

The behavior determination unit 236 generates the behavior content of the robot by adding a fixed sentence for inquiry about the behavior content of the robot corresponding to the behavior of the user to a text representing the behavior of the user, the emotion of the user, and the emotion of the robot, and inputting the text to the sentence generation model having the dialogue function.

For example, the behavior determination unit 236 acquires a text representing the state of the robot 100 from the emotion of the robot 100 determined by the emotion determination unit 232 using an emotion table as shown in Table 1. Here, in the emotion table, an index number is assigned to each emotion value for each type of emotion, and the text representing the state of the robot 100 is stored for each index number.

In a case where the emotion of the robot 100 determined by the emotion determination unit 232 corresponds to an index number “2”, a text “very pleasant state” is obtained. In a case where the emotion of the robot 100 corresponds to a plurality of index numbers, a plurality of texts representing the states of the robot 100 are obtained.

Further, an emotion table as shown in Table 2 is prepared for the emotion of the user 10.

Here, in a case where the behavior of the user is a behavior of saying “Should I head that way?”, the emotion of the robot 100 corresponds to the index number “2”, and the emotion of the user 10 corresponds to an index number “3”, a text “The robot is in a very pleasant state. The user is in a normally pleasant state. The user said, “Should I head that way?”. How should the robot respond?”
is input to the sentence generation model to thereby acquire the behavior content of the robot. The behavior determination unit 236 determines the behavior of the robot based on the behavior content.

TABLE 1

| | Type of | | |
|---|---|---|---|
| Index number | emotion | Emotion value | State of robot |
| 1 | Pleasant | 5 | Extremely pleasant state |
| 2 | Pleasant | 4 | Very pleasant state |
| 3 | Pleasant | 3 | Normally pleasant state |
| 4 | Pleasant | 2 | Slightly pleasant state |
| 5 | Pleasant | 1 | Faintly pleasant state |
| . . . | . . . | . . . | . . . |

TABLE 2

| | Type of | | |
|---|---|---|---|
| Index number | emotion | Emotion value | State of user |
| 1 | Pleasant | 5 | Extremely pleasant state |
| 2 | Pleasant | 4 | Very pleasant state |
| 3 | Pleasant | 3 | Normally pleasant state |
| 4 | Pleasant | 2 | Slightly pleasant state |
| 5 | Pleasant | 1 | Faintly pleasant state |
| . . . | . . . | . . . | . . . |

As described above, the behavior determination unit 236 determines the behavior content of the robot 100 according to a state related to the emotion of the robot 100 set in advance for each type of the emotion of the robot 100 and for each intensity of the emotion, and the behavior of the user 10. In the embodiment, the utterance content of the robot 100 in a case where a dialogue with the user 10 is performed can be branched according to the state related to the emotion of the robot 100. That is, since the robot 100 can change the behavior of the robot according to the index number corresponding to the emotion of the robot, the user is given an impression that the robot has a mind, and is promoted to perform a behavior such as talking to the robot.

Further, the behavior determination unit 236 may generate the behavior content of the robot by adding the fixed sentence for inquiry about the behavior content of the robot corresponding to the behavior of the user after adding not only the text representing the behavior of the user, the emotion of the user, and the emotion of the robot but also a text representing a content of the history data 222, and inputting the fixed sentence to the sentence generation model having the dialogue function. As a result, the robot 100 can change the behavior of the robot according to the history data indicating the emotion and the behavior of the user, and thus, the user is given an impression that the robot has a personality, and is promoted to perform a behavior such as talking to the robot. Further, the history data may further include the emotion and the behavior of the robot.

Further, the emotion determination unit 232 may determine the emotion of the robot 100 based on the behavior content of the robot 100 generated by the sentence generation model. Specifically, the emotion determination unit 232 inputs the behavior content of the robot 100 generated by the sentence generation model to the neural network trained in advance, acquires the emotion value indicating each emotion indicated in the emotion map 400, integrates the acquired emotion value indicating each emotion and the emotion value indicating each emotion of the current robot 100, and updates the emotion of the robot 100. For example, the acquired emotion value indicating each emotion and the current emotion value indicating each emotion of the robot 100 are each averaged and integrated. The neural network is learned in advance based on a plurality of pieces of learning data, which are a combination of the text representing the behavior content of the robot 100 generated by the sentence generation model and the emotion value representing each emotion indicated in the emotion map 400.

For example, in a case where an utterance content of the robot 100, “That's great. You were lucky”, is obtained as the behavior content of the robot 100 generated by the sentence generation model, when a text representing the utterance content is input to the neural network, a large value is obtained as the emotion value of the emotion “joyful”, and the emotion of the robot 100 is updated such that the emotion value of the emotion “joyful” becomes large.

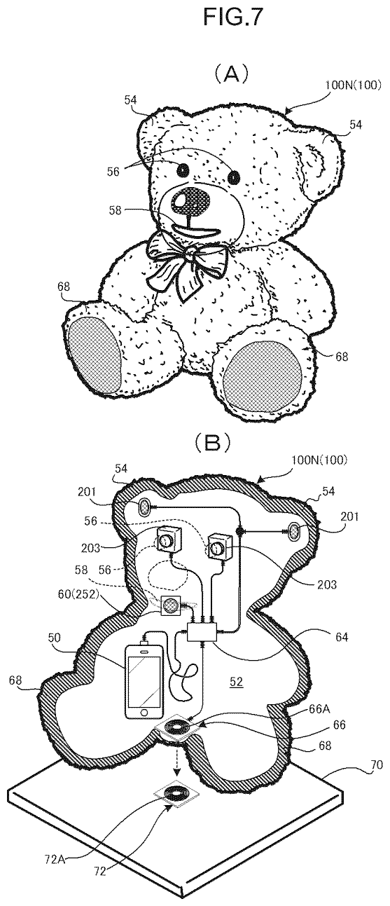

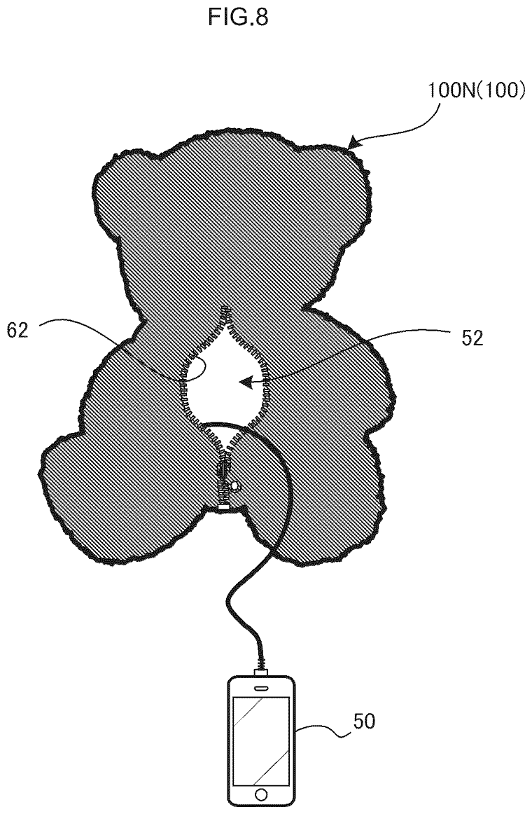

The robot 100 may be mounted on a stuffed toy, or may be applied to a control device connected wirelessly or by wire to control target equipment (speaker or camera) mounted on a stuffed toy. In this case, specifically, the following configuration is applied. For example, the robot 100 may be applied to a cohabiting companion (specifically, a stuffed toy 100N shown in FIGS. 7 and 8) that has a dialogue with the user 10 based on information regarding daily life and provides information tailored to preferences of the user 10 while spending daily life with the user 10. In the present embodiment (another embodiment), an example in which a control portion of the robot 100 is applied to the smartphone 50 is described.

The smartphone 50 functioning as the control portion of the robot 100 is attachable to and detachable from the stuffed toy 100N having a function as an input/output device of the robot 100, and the input/output device and the housed smartphone 50 are connected inside the stuffed toy 100N.

As shown in FIG. 7(A), the stuffed toy 100N has a shape of a bear covered with a soft cloth fabric in the present embodiment (an embodiment in which the robot 100 is mounted on the stuffed toy), and as shown in FIG. 7(B), in a space portion 52 formed inside the stuffed toy 100N, the microphone 201 (see FIG. 2) of the sensor unit 200 is disposed as the input/output device at a portion corresponding to an ear 54, the 2D camera 203 (see FIG. 2) of the sensor unit 200 is disposed at a portion corresponding to an eye 56, and a speaker 60 forming a part of the control target 252 (see FIG. 2) is disposed at a portion corresponding to a mouth 58. The microphone 201 and the speaker 60 are not necessarily separated from each other, and may be formed as an integrated unit. In a case where the microphone 201 and the speaker 60 are formed as the unit, it is preferable to dispose the unit at a position where an utterance can be heard naturally, such as a position of a nose of the stuffed toy 100N. Although a case where the stuffed toy 100N has an animal shape has been described as an example, the disclosure is not limited thereto. The stuffed toy 100N may have a shape of a specific character.

The smartphone 50 has a function as the sensor module unit 210, a function as the storage unit 220, a function as the user state recognition unit 230, a function as the emotion determination unit 232, a function as the behavior recognition unit 234, a function as the behavior determination unit 236, a function as the storage control unit 238, a function as the behavior control unit 250, and a function as the communication processing unit 280 shown in FIG. 2.

As shown in FIG. 8, a fastener 62 is attached to a part (for example, a back portion) of the stuffed toy 100N, and the outside and the space portion 52 communicate with each other by opening the fastener 62.

Here, the smartphone 50 is housed in the space portion 52 from the outside and is universal serial bus (USB)-connected to each input/output device via a USB hub 64 (see FIG. 7(B)), so that functions equivalent to those of the robot 100 shown in FIG. 1 can be provided.

A non-contact power receiving plate 66 is connected to the USB hub 64. A power receiving coil 66A is incorporated in the power receiving plate 66. The power receiving plate 66 is an example of a wireless power receiving unit that receives wireless power supply.

The power receiving plate 66 is disposed near root portions 68 of both feet of the stuffed toy 100N and is positioned closest to a placement base 70 in a case where the stuffed toy 100N is placed on the placement base 70. The placement base 70 is an example of an external wireless power transmitting unit.

The stuffed toy 100N placed on the placement base 70 can be appreciated as an ornament in a natural state.

Further, the root portion is formed to have a thickness smaller than a thickness of a surface layer of the stuffed toy 100N at other portions, and is held in a state closer to the placement base 70.

The placement base 70 includes a charging pad 72. A power transmitting coil 72A is incorporated in the charging pad 72. When the power transmitting coil 72A transmits a signal to search the power receiving coil 66A of the power receiving plate 66, and the power receiving coil 66A is found, a current flows through the power transmitting coil 72A to generate a magnetic field, and the power receiving coil 66A reacts to the magnetic field to start electromagnetic induction. As a result, a current flows through the power receiving coil 66A, and power is stored in a battery (not shown) of the smartphone 50 via the USB hub 64.

That is, since the smartphone 50 is automatically charged by placing the stuffed toy 100N as an ornament on the placement base 70, it is not necessary to take out the smartphone 50 from the space portion 52 of the stuffed toy 100N for charging.

In the present embodiment (an embodiment in which the robot 100 is mounted on the stuffed toy), the smartphone 50 is housed in the space portion 52 of the stuffed toy 100N and connected by wire (USB connection), but the disclosure is not limited thereto. For example, a control device having a wireless function (for example, “Bluetooth (registered trademark)”) may be housed in the space portion 52 of the stuffed toy 100N, and the control device may be connected to the USB hub 64. In this case, the smartphone 50 and the control device wirelessly communicate with each other in a state in which the smartphone 50 is not inserted into the space portion 52, and the smartphone 50 positioned outside is connected to each input/output device via the control device, so that functions equivalent to those of the robot 100 shown in FIG. 1 can be provided. Further, the control device in which the control device is housed in the space portion 52 of the stuffed toy 100N and the smartphone 50 positioned outside may be connected by wire.

Further, in the present embodiment (an embodiment in which the robot 100 is mounted on the stuffed toy), the bear-shaped stuffed toy 100N has been exemplified, but the shape of the stuffed toy 100N may be another animal, a doll, or a shape of a specific character. Further, clothes of the stuffed toy 100N may be able to be changed. Further, a material of an outer surface is not limited to the cloth fabric and may be other materials such as soft vinyl. It is preferable that the material of the outer surface is a soft material.

Further, a monitor may be attached to the outer surface of the stuffed toy 100N, and the control target 252 that provides information to the user 10 through vision may be added. For example, the eye 56 may be used as the monitor to express joy, anger, sorrow, and pleasure, or a window through which a built-in monitor of the smartphone 50 is visible may be provided at a belly portion. Further, the eye 56 may be used as a projector to express joy, anger, sorrow, and pleasure by an image projected on a wall surface.

According to another embodiment, the existing smartphone 50 is inserted into the stuffed toy 100N, and the camera 203, the microphone 201, the speaker 60, and the like are extended from the smartphone 50 to appropriate positions via USB connection.

Further, for wireless charging, the smartphone 50 and the power receiving plate 66 are USB-connected to each other, and the power receiving plate 66 is disposed as close to the outer side of the stuffed toy 100N as possible when viewed from the inside.

In order to use the wireless charging of the smartphone 50, the smartphone 50 needs to be positioned as close to the outer side of the stuffed toy 100N as possible when viewed from the inside, which may result in a rough tactile sensation when the stuffed toy 100N is touched from the outside.

Therefore, the smartphone 50 is disposed as close to the center of the stuffed toy 100N as possible, and a wireless charging function (power receiving plate 66) is disposed as close to the outer side of the stuffed toy 100N as possible when viewed from the inside. The camera 203, the microphone 201, the speaker 60, and the smartphone 50 receive wireless power supply via the power receiving plate 66.

### Another Embodiment 2

A behavior system for the robot 100 according to the present embodiment is characterized to include the emotion determination unit 232 that determines emotions of users 10, 11, and 12 or the emotion of the robot 100, and the behavior determination unit 236 that generates the behavior content of the robot 100 for the behavior of the user and the emotions of the users 10, 11, and 12 or the emotion of the robot 100 based on the dialogue function that causes the users 10, 11, and 12 and the robot 100 to have a dialogue with each other, and determines the behavior of the robot 100 corresponding to the behavior content, in which in a case where it is determined that the users 10, 11, and 12 are specific users including an individual who lives alone in isolation, the behavior determination unit 236 switches to a specific mode in which the behavior of the robot is determined based on a communication count larger than a communication count in a normal mode in which the behavior is determined for the users 10, 11, and 12 other than the specific user.

The behavior determination unit 236 can set a specific mode separately from the normal mode and cause the specific mode to function as a support for an elderly person living alone. In other words, in a case where the robot 100 detects a situation of the user and determines that the user is a user living alone since a spouse of the user has passed away or a child of the user has left home, the behavior determination unit 236 makes a gesture and an utterance for the user more actively than in the normal mode, and increases the communication count between the user and the robot 100 (switching to the specific mode).

The communication includes, in addition to a dialogue, a special response to the specific user, such as a confirmation behavior in which the robot 100 intentionally makes a change in daily life (for example, turning off the light, sounding an alarm, or the like) and confirms a response behavior for the change in daily life, and the confirmation behavior is also counted as the communication. The confirmation behavior can be referred to as an indirect communication behavior.

In addition, in a case where there is no conversation with the robot 100 for a certain period of time, a preset emergency contact is contacted.

With the elderly-living-alone support function, the robot 100 becomes a conversation partner for the elderly person who lives alone because the spouse has passed away or the child has left home. Such a function also prevents cognitive decline. It is also possible to contact the preset emergency contact in a case where there is no conversation with the robot 100 for a certain period of time.

Not only for the elderly person, but also for the individual who lives alone in isolation, it is effective to set the individual as a target user (specific user) of the elderly-living-alone support function.

### Another Embodiment 3

The behavior determination unit 236 acquires a result of summarizing minutes of the past meeting held at a meeting room, and in a case where a statement whose content is similar to the summarized minutes has been made in a new meeting different from the past meeting, the behavior determination unit 236 determines, as the behavior of the robot 100, outputting of advice information for the statement.

Specifically, the robot 100 is installed in the meeting room. Then, the robot 100 summarizes the minutes of the meeting held at the meeting room by using the sentence generation model. The summarization of the minutes is not limited to the use of the sentence generation model, and may be performed using other known methods. The summarized minutes are stored in the robot 100. Then, in a case where the behavior determination unit 236 recognizes that a participant has made a statement similar to the stored minutes in a new meeting held at the meeting room, the behavior determination unit 236 determines, as the behavior of the robot 100, outputting of the advice information to the participant of the meeting. Here, determination of a similarity of an utterance is made using, for example, a known method of converting the utterance into a vector (quantifying the utterance) and calculating a similarity between vectors, and may be performed using other methods. Furthermore, examples of the advice information include “That content was already presented by [person] on [date]”, which indicates that there has been a statement having a similar content in the past meeting, and “That content is superior to the content proposed by [person] in this respect” which indicates a result of comparison with a statement having a similar content in the past meeting.

### Second Embodiment

FIG. 1 schematically shows an example of a system 5 according to the present embodiment. The system 5 includes a robot 100, a robot 101, a robot 102, and a server 300. A user 10a, a user 10b, a user 10c, and a user 10d are users of the robot 100. A user 11a, a user 11b, and a user 11c are users of the robot 101. A user 12a and a user 12b are users of the robot 102. In the description of the present embodiment, the user 10a, the user 10b, the user 10c, and the user 10d may be collectively referred to as the user 10. Further, the user 11a, the user 11b, and the user 11c may be collectively referred to as the user 11. Further, the user 12a and the user 12b may be collectively referred to as the user 12. The robot 101 and the robot 102 have substantially the same functions as that of the robot 100. Therefore, the system 5 will be described focusing on the function of the robot 100.

The robot 100 has a conversation with the user 10 and provides a video to the user 10. At this time, the robot 100 has a conversation with the user 10, provides a video to the user 10, and the like in cooperation with the server 300 and the like that can perform communication via a communication network 20. For example, the robot 100 not only learns an appropriate conversation by itself, but also performs learning to have a more appropriate conversation with the user 10 in cooperation with the server 300. Further, the robot 100 causes the server 300 to record captured video data and the like of the user 10, requests the server 300 to transmit the video data and the like if necessary, and provides the video data and the like to the user 10.

Further, the robot 100 has an emotion value representing a type of an emotion thereof. For example, the robot 100 has the emotion value representing an intensity of each of emotions “joy”, “anger”, “sorrow”, “pleasure”, “comfort”, “discomfort”, “relief”, “anxiety”, “sadness”, “excitement”, “worry”, “reassurance”, “sense of fulfillment”, “sense of emptiness”, and “neutral”. For example, in the case of having a conversation with the user 10 in a state in which the emotion value of excitement is large, the robot 100 utters a speech at a high speed. As described above, the robot 100 can express the emotion thereof by a behavior.

Further, the robot 100 may be configured to determine a behavior of the robot 100 corresponding to an emotion of the user 10 by matching a sentence generation model and an emotion engine using an artificial intelligence (AI). Specifically, the robot 100 may be configured to recognize a behavior of the user 10, determine the emotion of the user 10 for the behavior of the user, and determine the behavior of the robot 100 corresponding to the determined emotion.

More specifically, in a case where the behavior of the user 10 is recognized, the robot 100 automatically generates a content of a behavior to be performed by the robot 100 for the behavior of the user 10 using the preset sentence generation model. The sentence generation model may be interpreted as an algorithm and operation for text-based automatic dialogue processing. Since the sentence generation model is known as disclosed in, for example, Japanese Patent Application Laid-Open No. 2018-081444 and ChatGPT (Internet search <URL: https://openai.com/blog/chatgpt>), a detailed description thereof is omitted.

Such a sentence generation model is implemented by a large language model (LLM).

As described above, in the present embodiment, it is possible to reflect the emotions of the user 10 and the robot 100 and various types of linguistic information in the behavior of the robot 100 by combining the large language model and the emotion engine. That is, according to the present embodiment, a synergistic effect can be obtained by combining the sentence generation model and the emotion engine.

Further, the robot 100 has a function of recognizing the behavior of the user 10. The robot 100 recognizes the behavior of the user 10 by analyzing a face image of the user 10 acquired by a camera function and a speech of the user 10 acquired by a microphone function. The robot 100 determines a behavior to be performed by the robot 100 based on the recognized behavior of the user 10 or the like.

The robot 100 stores, as an example of a behavior determination model, a rule setting a behavior to be performed by the robot 100 based on the emotion of the user 10, the emotion of the robot 100, and the behavior of the user 10, and performs various behaviors according to the rule.

Specifically, the robot 100 has, as an example of the behavior determination model, a reaction rule for determining the behavior of the robot 100 based on the emotion of the user 10, the emotion of the robot 100, and the behavior of the user 10. In the reaction rule, for example, a behavior of “laughing” is set as the behavior of the robot 100 for a case where the behavior of the user 10 is “laughing”. Further, in the reaction rule, a behavior of “apologizing” is set as the behavior of the robot 100 for a case where the behavior of the user 10 is “getting angry”. Further, in the reaction rule, a behavior of “answering” is set as the behavior of the robot 100 for a case where the behavior of the user 10 is “asking a question”. In the reaction rule, a behavior of “calling out” is set as the behavior of the robot 100 for a case where the behavior of the user 10 is “being sad”.

In a case where the robot 100 recognizes that the behavior of the user 10 is “getting angry”, the robot 100 selects the behavior of “apologizing” set in the reaction rule as a behavior to be performed by the robot 100 based on the reaction rule. For example, in a case where the behavior of “apologizing” is selected, the robot 100 performs the behavior of “apologizing” and outputs a speech representing words of “apology”.

Further, in a case where a condition that the emotion of the robot 100 is “neutral” (that is, “joy”=0, “anger”=0, “sorrow”=0, and “pleasure”=0) and a state of the user 10 is “alone and looking lonely” is satisfied, a content of a change in the emotion of the robot 100 to “worried” is determined, and it is determined that the behavior of “calling out” can be performed.

In a case where the robot 100 recognizes that the current emotion of the robot 100 is “neutral” and the user 10 is alone and looks lonely, the emotion value of “sorrow” of the robot 100 is increased based on the reaction rule. Further, the robot 100 selects the behavior of “calling out” set in the reaction rule as a behavior to be performed for the user 10. For example, in a case where the behavior of “calling out” is selected, the robot 100 converts a phrase “What's wrong?” expressing that the robot 100 is worried into a sympathetic voice, and outputs the voice.

Further, the robot 100 transmits, to the server 300, user reaction information indicating that a positive reaction has been obtained from the user 10 for the behavior. Examples of the user reaction information include the user behavior of “getting angry”, the behavior of the robot 100 of “apologizing”, the positive reaction of the user 10, and an attribute of the user 10.

The server 300 stores the user reaction information received from the robot 100.

The server 300 receives and stores the user reaction information not only from the robot 100 but also from each of the robot 101 and the robot 102. Then, the server 300 analyzes the user reaction information from the robot 100, the robot 101, and the robot 102, and updates the reaction rule.

The robot 100 receives the updated reaction rule from the server 300 by inquiring the server 300 about the updated reaction rule. The robot 100 incorporates the updated reaction rule into the reaction rule stored in the robot 100. As a result, the robot 100 can incorporate the reaction rule acquired by the robot 101, the robot 102, or the like into the reaction rule thereof.

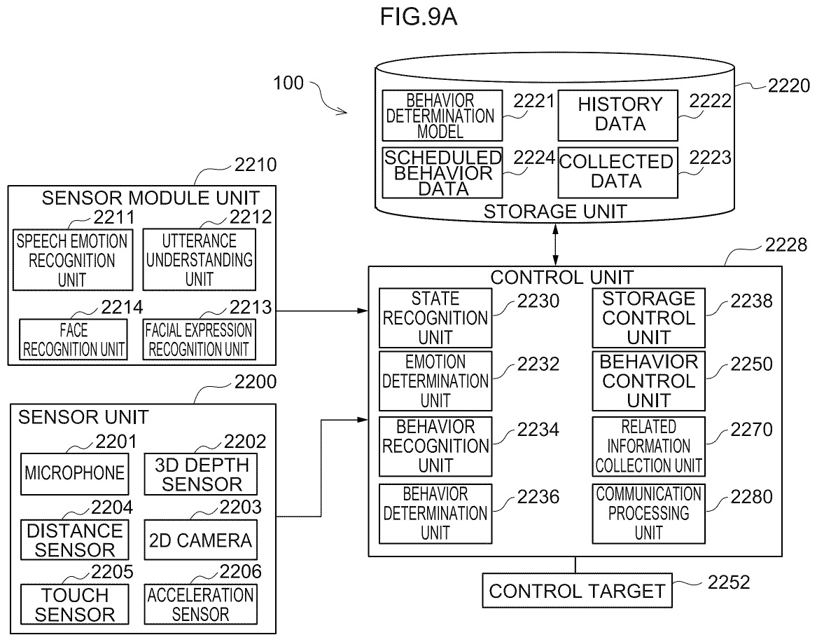

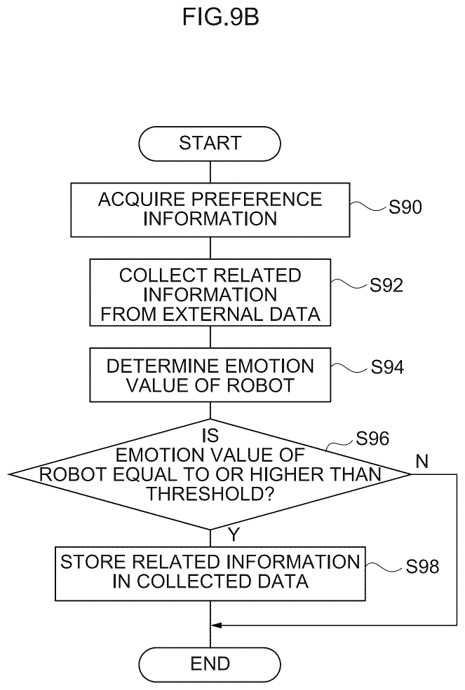

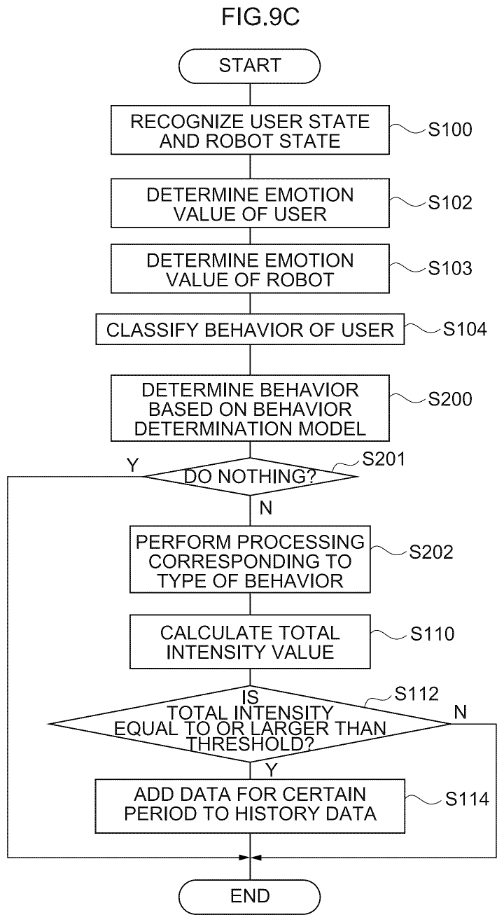

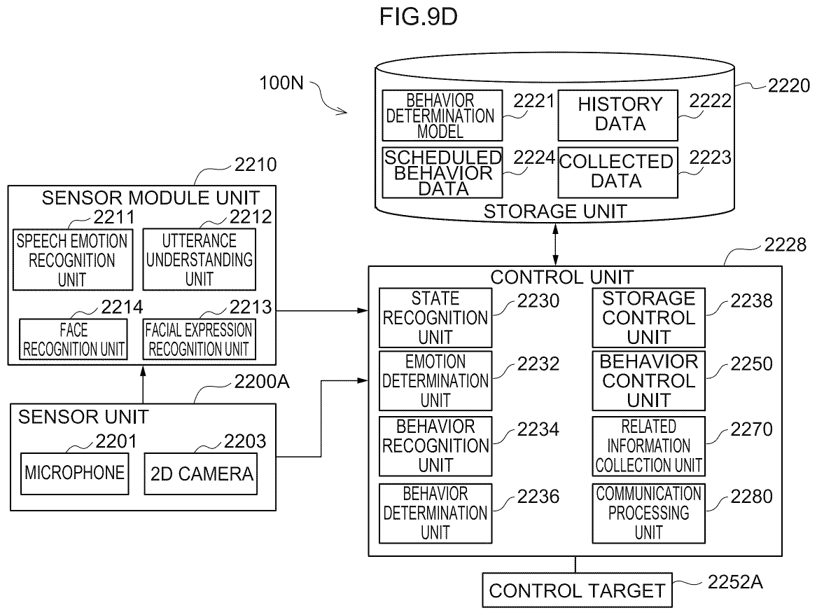

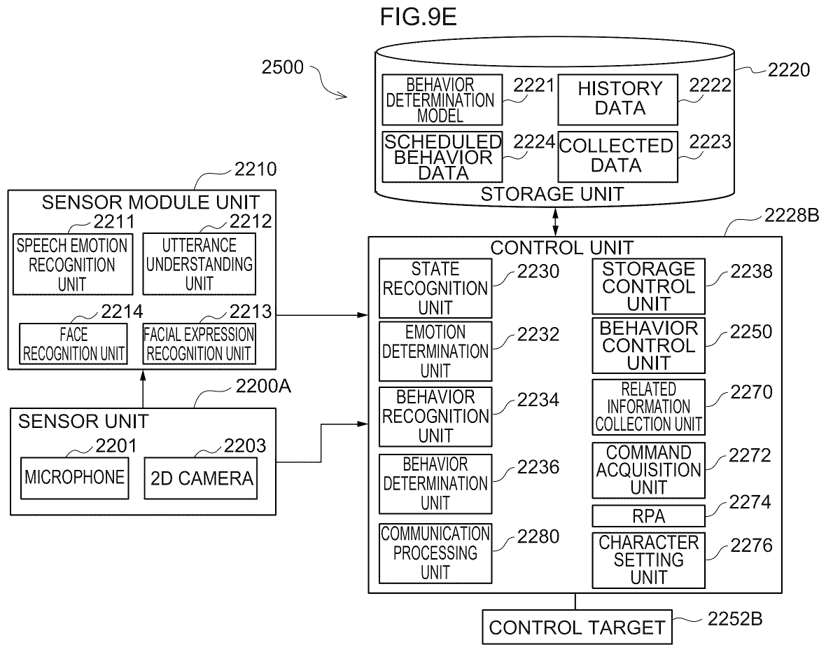

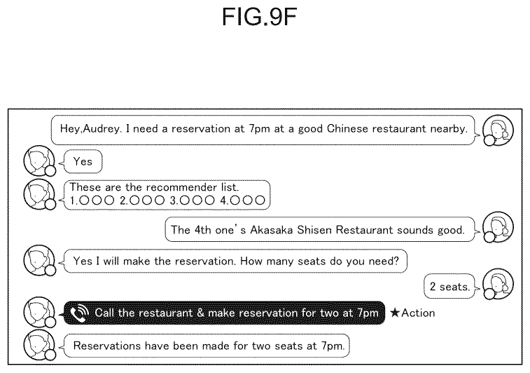

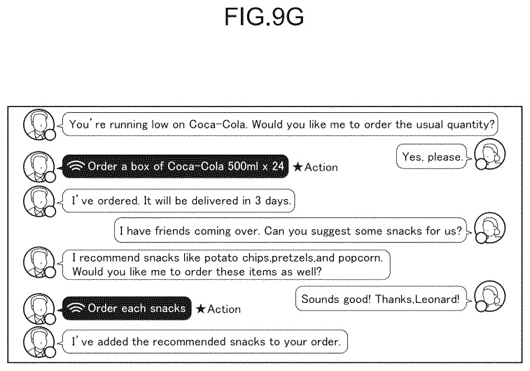

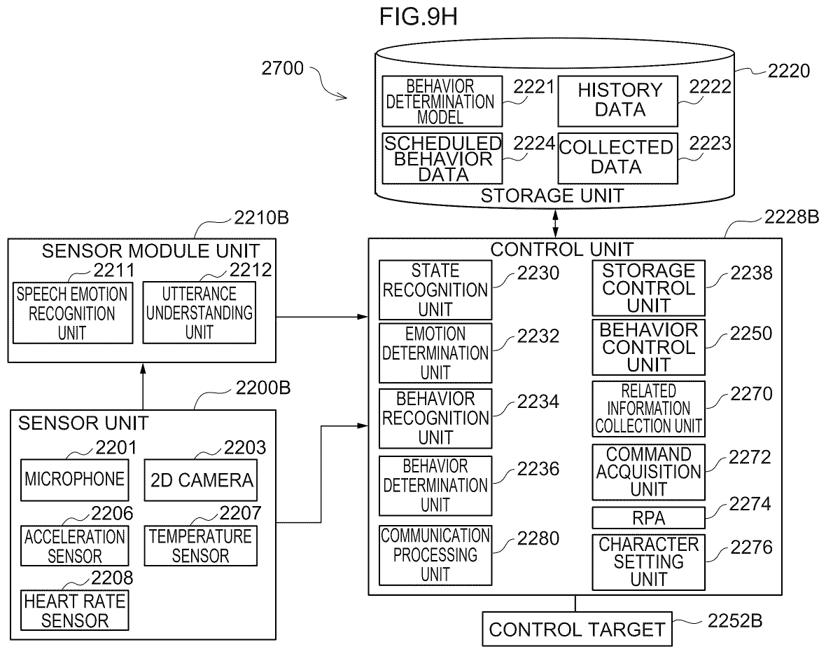

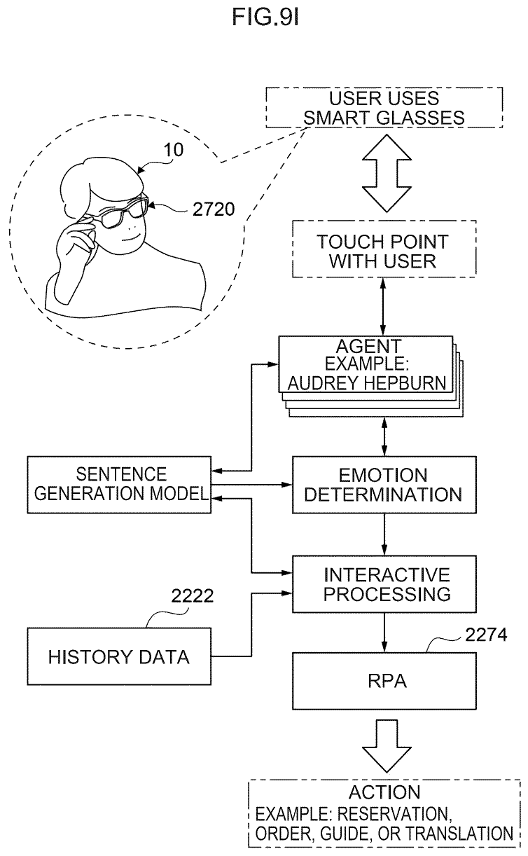

FIG. 9A schematically shows a functional configuration of the robot 100. The robot 100 includes a sensor unit 2200, a sensor module unit 2210, a storage unit 2220, a control unit 2228, and a control target 2252. The control unit 2228 includes a state recognition unit 2230, an emotion determination unit 2232, a behavior recognition unit 2234, a behavior determination unit 2236, a storage control unit 2238, a behavior control unit 2250, a related information collection unit 2270, and a communication processing unit 2280.

The control target 2252 includes a display device, a speaker, a light emitting diode (LED) of an eye portion, motors that drive an arm, a hand, a foot, and the like, and the like. A posture and a gesture of the robot 100 are controlled by controlling the motors for the arm, the hand, the foot, and the like. Some emotions of the robot 100 can be expressed by controlling the motors. Furthermore, a facial expression of the robot 100 can be expressed by controlling a light emission state of the LED of the eye portion of the robot 100. The posture, the gesture and the facial expression of the robot 100 are examples of an attitude of the robot 100.

The sensor unit 2200 includes a microphone 2201, a 3D depth sensor 2202, a 2D camera 2203, a distance sensor 2204, a touch sensor 2205, and an acceleration sensor 2206. The microphone 2201 continuously detects a speech and outputs speech data. The microphone 2201 may be provided at a head portion of the robot 100 and may have a function of performing binaural recording. The 3D depth sensor 2202 detects an outline of an object by continuously radiating an infrared pattern and analyzing the infrared pattern based on an infrared image continuously captured by an infrared camera. The 2D camera 2203 is an example of an image sensor. The 2D camera 2203 performs imaging with visible light and generates video information of visible light. The distance sensor 2204 detects a distance to an object by emitting, for example, a laser beam or an ultrasonic wave. The sensor unit 2200 may further include a clock, a gyro sensor, a sensor for motor feedback, and the like.

Among the components of the robot 100 shown in FIG. 9A, the components other than the control target 2252 and the sensor unit 2200 are examples of components included in a behavior control system included in the robot 100. The behavior control system of the robot 100 controls the control target 2252.

The storage unit 2220 includes a behavior determination model 2221, history data 2222, collected data 2223, and scheduled behavior data 2224. The history data 2222 includes a history of the past emotion value of a user 10, the past emotion value of the robot 100, and behaviors, and specifically includes a plurality of pieces of event data including an emotion value of the user 10, an emotion value of the robot 100, and the behavior of the user 10. Data including the behavior of the user 10 includes a camera image representing the behavior of the user 10. The history of the emotion value and the behavior is recorded for each user 10 by being associated with identification information of the user 10, for example. At least a part of the storage unit 2220 is implemented by a storage medium such as a memory. A person DB that stores a face image of the user 10, attribute information of the user 10, and the like may be included. Among the components of the robot 100 shown in FIG. 9A, functions of the components other than the control target 2252, the sensor unit 2200, and the storage unit 2220 can be implemented by a CPU operating based on a program. For example, the functions of the components can be implemented as an operation of the CPU by basic software (operating system (OS)) and a program operating on the OS.

The sensor module unit 2210 includes a speech emotion recognition unit 2211, an utterance understanding unit 2212, a facial expression recognition unit 2213, and a face recognition unit 2214. Information detected by the sensor unit 2200 is input to the sensor module unit 2210. The sensor module unit 2210 analyzes the information detected by the sensor unit 2200 and outputs an analysis result to the state recognition unit 2230.

The speech emotion recognition unit 2211 of the sensor module unit 2210 analyzes a speech of the user 10 detected by the microphone 2201 to recognize the emotion of the user 10. For example, the speech emotion recognition unit 2211 extracts a feature amount such as a frequency component of a speech and recognizes the emotion of the user 10 based on the extracted feature amount. The utterance understanding unit 2212 analyzes the speech of the user 10 detected by the microphone 2201 and outputs text information indicating an utterance content of the user 10.

The facial expression recognition unit 2213 recognizes a facial expression of the user 10 and the emotion of the user 10 from an image of the user 10 captured by the 2D camera 2203. For example, the facial expression recognition unit 2213 recognizes the facial expression and the emotion of the user 10 based on shapes, positional relationships, and the like of the eyes and the mouth.

The face recognition unit 2214 recognizes the face of the user 10. The face recognition unit 2214 recognizes the user 10 by matching a face image stored in the person DB (not shown) with a face image of the user 10 captured by the 2D camera 2203.

The state recognition unit 2230 recognizes a state of the user 10 based on the information analyzed by the sensor module unit 2210. For example, processing mainly related to perception is performed using an analysis result of the sensor module unit 2210. For example, perception information such as “Dad is alone” and “There is a 90% probability that dad is not smiling” is generated. Processing of understanding the meaning of the generated perception information is performed. For example, semantic information such as “Dad is alone and looks lonely” is generated.

The state recognition unit 2230 recognizes a state of the robot 100 based on the information detected by the sensor unit 2200. For example, the state recognition unit 2230 recognizes a remaining battery level of the robot 100, a brightness of a surrounding environment of the robot 100, and the like as the state of the robot 100.

The emotion determination unit 2232 determines an emotion value indicating the emotion of the user 10 based on the information analyzed by the sensor module unit 2210 and the state of the user 10 recognized by the state recognition unit 2230. For example, the emotion value indicating the emotion of the user 10 is acquired by inputting the information analyzed by the sensor module unit 2210 and the recognized state of the user 10 to a neural network trained in advance.

Here, the emotion value indicating the emotion of the user 10 is a value indicating whether the emotion of the user is positive or negative. For example, the emotion value has a positive value in a case where the emotion of the user is a bright emotion accompanied by pleasure or a sense of calm, such as “joy”, “pleasure”, “comfort”, “relief”, “excitement”, “reassurance”, or “sense of fulfillment”, and the emotion value becomes larger as the emotion becomes brighter. The emotion value has a negative value in a case where the emotion of the user is an unpleasant emotion such as “anger”, “sorrow”, “discomfort”, “anxiety”, “sadness”, “worry”, or “sense of emptiness”, and the more unpleasant the emotion is, the larger the absolute value of the negative value becomes. In a case where the emotion of the user is not any of the above (“neutral”), the emotion value has a value of 0.

Further, the emotion determination unit 2232 determines an emotion value indicating the emotion of the robot 100 based on the information analyzed by the sensor module unit 2210, the information detected by the sensor unit 2200, and the state of the user 10 recognized by the state recognition unit 2230.

The emotion value of the robot 100 includes an emotion value for each of a plurality of emotion classifications, and is, for example, a value (0 to 5) indicating an intensity of each of “joy”, “anger”, “sorrow”, and “pleasure”.

Specifically, the emotion determination unit 2232 determines the emotion value indicating the emotion of the robot 100 according to a rule for updating the emotion value of the robot 100, the rule being set in association with the information analyzed by the sensor module unit 2210 and the state of the user 10 recognized by the state recognition unit 2230.

For example, in a case where the state recognition unit 2230 recognizes that the user 10 looks lonely, the emotion determination unit 2232 increases the emotion value of “sorrow” of the robot 100. Furthermore, in a case where the state recognition unit 2230 recognizes that the user 10 is smiling, the emotion determination unit 2232 increases the emotion value of “joy” of the robot 100.

The emotion determination unit 2232 may determine the emotion value indicating the emotion of the robot 100 in further consideration of a state of the robot 100. For example, in a case where the remaining battery level of the robot 100 is low, a case where the surrounding environment of the robot 100 is dark, or the like, the emotion determination unit 232 may increase the emotion value of “sorrow” of the robot 100. Furthermore, in the case of the user 10 who continues to speak to the robot 100 despite the low remaining battery level, the emotion determination unit 232 may increase the emotion value of “anger”.

The behavior recognition unit 2234 recognizes the behavior of the user 10 based on the information analyzed by the sensor module unit 2210 and the state of the user 10 recognized by the state recognition unit 2230. For example, a probability of each of a plurality of predetermined behavior classifications (for example, “laughing”, “getting angry”, “asking a question”, and “being sad”) is acquired by inputting the information analyzed by the sensor module unit 2210 and the recognized state of the user 10 to the neural network trained in advance, and a behavior classification having the highest probability is recognized as the behavior of the user 10.

As described above, in the present embodiment, the robot 100 acquires an utterance content of the user 10 after specifying the user 10, but in acquiring and using the utterance content, the behavior control system of the robot 100 according to the present embodiment considers protection of personal information and privacy of the user 10 in addition to acquisition of necessary consent according to laws and regulations from the user 10.

Next, processing performed by the behavior determination unit 2236 in a case where the robot 100 performs response processing of responding to the behavior of the user 10 will be described.

The behavior determination unit 2236 determines a behavior corresponding to the behavior of the user 10 recognized by the behavior recognition unit 2234, based on the current emotion value of the user 10 determined by the emotion determination unit 2232, the history data 2222 of the past emotion value determined by the emotion determination unit 2232 before the current emotion value of the user 10 is determined, and the emotion value of the robot 100. In the present embodiment, a case where the behavior determination unit 2236 uses one most recent emotion value included in the history data 2222 as the past emotion value of the user 10 is described, but the disclosed technology is not limited to such an aspect. For example, the behavior determination unit 2236 may use a plurality of most recent emotion values as the past emotion values of the user 10, or may use emotion values from a unit period earlier, such as one day ago, as the past emotion values of the user 10. Further, the behavior determination unit 2236 may determine the behavior corresponding to the behavior of the user 10 in further consideration of the history of the past emotion value of the robot 100 in addition to the current emotion value of the robot 100. The behavior determined by the behavior determination unit 2236 includes the gesture made by the robot 100 or an utterance content of the robot 100.

The behavior determination unit 2236 according to the present embodiment determines, as the behavior corresponding to the behavior of the user 10, the behavior of the robot 100 based on a combination of the past emotion value and the current emotion value of the user 10, the emotion value of the robot 100, the behavior of the user 10, and the behavior determination model 2221. For example, in a case where the past emotion value of the user 10 is a positive value and the current emotion value is a negative value, the behavior determination unit 2236 determines a behavior for positively changing the emotion value of the user 10 as the behavior corresponding to the behavior of the user 10.

In a reaction rule as the behavior determination model 2221, the behavior of the robot 100 based on a combination of the past emotion value and the current emotion value of the user 10, the emotion value of the robot 100, and the behavior of the user 10 is set. For example, a combination of a gesture and an utterance content when encouraging the user 10 with a gesture is set as the behavior of the robot 100 in a case where the past emotion value of the user 10 is a positive value, the current emotion value is a negative value, and the behavior of the user 10 is being sad.

For example, in the reaction rule as the behavior determination model 2221, behaviors of the robot 100 are set for all combinations of patterns of the emotion value of the robot 100 (1296 patterns which correspond to the fourth power of six values of “0” to “5” of “joy”, “anger”, “sorrow”, and “pleasure”), patterns of a combination of the past emotion value and the current emotion value of the user 10, and a behavior pattern of the user 10. That is, for each pattern of the emotion value of the robot 100, the behavior of the robot 100 based on the behavior pattern of the user 10 is determined for each of a plurality of combinations of the past emotion value and the current emotion value of the user 10, such as a combination of a negative value and a negative value, a combination of a negative value and a positive value, a combination of a positive value and a negative value, a combination of a positive value and a positive value, a combination of a negative value and a value indicating the neutral emotion, and a combination of a value indicating the neutral emotion and a value indicating the neutral emotion. The behavior determination unit 2236 may transition to an operation mode of determining the behavior of the robot 100 by using the history data 2222, for example, in a case where the user 10 has made an utterance that intends to continue a conversation of the past topic, such as “I want to talk about the topic we discussed earlier”.

In the reaction rule as the behavior determination model 2221, at least one of a gesture and a statement content may be set as the behavior of the robot 100 for each pattern (1296 patterns) of the emotion value of the robot 100, with at most one behavior per pattern. Alternatively, in the reaction rule as the behavior determination model 2221, at least one of the gesture and the statement content may be set as the behavior of the robot 100 for each group of the patterns of the emotion values of the robot 100.

An intensity of each gesture included in the behavior of the robot 100 and set in the reaction rule as the behavior determination model 2221 is set in advance. An intensity of each utterance content included in the behavior of the robot 100 set in the reaction rule as the behavior determination model 2221 is set in advance.

The storage control unit 2238 determines whether or not to store data including the behavior of the user 10 in the history data 2222 based on a predetermined intensity of the behavior for the behavior determined by the behavior determination unit 2236 and the emotion value of the robot 100 determined by the emotion determination unit 2232.

Specifically, in a case where the total sum of the emotion values of the plurality of emotion classifications of the robot 100 and a total intensity value, which is the sum of the predetermined intensity for the gesture included in the behavior determined by the behavior determination unit 2236 and the predetermined intensity for the utterance content included in the behavior determined by the behavior determination unit 2236, are equal to or larger than thresholds, the storage control unit 2238 determines to store the data including the behavior of the user 10 in the history data 2222.

In a case where the storage control unit 2238 determines to store the data including the behavior of the user 10 in the history data 2222, the behavior determined by the behavior determination unit 2236, the information (for example, any surrounding information such as data such as a sound, an image, and a scent at that time) analyzed by the sensor module unit 2210 over a certain period prior to the current time point, and the state (for example, the facial expression or emotion of the user 10) of the user 10 recognized by the state recognition unit 2230 are stored in the history data 2222.

The behavior control unit 2250 controls the control target 2252 based on the behavior determined by the behavior determination unit 2236. For example, in a case where the behavior determination unit 2236 determines a behavior including an utterance, the behavior control unit 2250 causes the speaker included in the control target 2252 to output a speech. At this time, the behavior control unit 2250 may determine an utterance speed of the speech based on the emotion value of the robot 100. For example, the behavior control unit 2250 determines a higher utterance speed as the emotion value of the robot 100 is larger. In this manner, the behavior control unit 2250 determines an execution mode of the behavior determined by the behavior determination unit 2236 based on the emotion value determined by the emotion determination unit 2232.

The behavior control unit 2250 may recognize a change in the emotion of the user 10 for execution of the behavior determined by the behavior determination unit 2236. For example, the change in the emotion may be recognized based on the speech or facial expression of the user 10. In addition, the change in the emotion of the user 10 may be recognized based on detection of an impact applied to the touch sensor 2205 included in the sensor unit 2200. In a case where an impact is detected by the touch sensor 2205 included in the sensor unit 2200, it may be recognized that the emotion of the user 10 has become worse, and in a case where it is determined that the reaction of the user 10 is smiling or being happy based on a detection result of the touch sensor 2205 included in the sensor unit 2200, it may be recognized that the emotion of the user 10 has been improved. Information indicating the reaction of the user 10 is output to the communication processing unit 2280.

Further, after the behavior control unit 2250 performs the behavior determined by the behavior determination unit 2236 in the execution mode determined according to the emotion of the robot 100, the emotion determination unit 2232 further changes the emotion value of the robot 100 based on the reaction of the user for the execution of the behavior. Specifically, the emotion determination unit 2232 increases the emotion value of “joy” of the robot 100 in a case where the reaction of the user for the behavior determined by the behavior determination unit 2236 and performed for the user in the execution form determined by the behavior control unit 2250 is not negative. Further, the emotion determination unit 2232 increases the emotion value of “sorrow” of the robot 100 in a case where the reaction of the user for the behavior determined by the behavior determination unit 2236 and performed for the user in the execution form determined by the behavior control unit 2250 is negative.

Furthermore, the behavior control unit 2250 expresses the emotion of the robot 100 based on the determined emotion value of the robot 100. For example, in a case where the emotion value of “joy” of the robot 100 is increased, the behavior control unit 2250 controls the control target 2252 to cause the robot 100 to make a joyful gesture. Further, in a case where the emotion value of “sorrow” of the robot 100 is increased, the behavior control unit 2250 controls the control target 2252 such that the posture of the robot 100 becomes a drooping posture.

The communication processing unit 2280 is responsible for communication with the server 300. As described above, the communication processing unit 2280 transmits the user reaction information to the server 300. Further, the communication processing unit 2280 receives the updated reaction rule from the server 300. In a case where the updated reaction rule is received from the server 300, the communication processing unit 2280 updates the reaction rule as the behavior determination model 2221.

The server 300 performs communication between the server 300 and the robot 100, the robot 101, and the robot 102, receives the user reaction information transmitted from the robot 100, and updates the reaction rule based on a reaction rule including a behavior for which a positive reaction has been obtained.

The related information collection unit 2270 collects information related to preference information from external data (web sites such as news sites and moving image sites) based on the preference information acquired for the user 10 at a predetermined timing.

Specifically, the related information collection unit 2270 acquires the preference information indicating matters of interest to the user 10 from the utterance content of the user 10 or a setting operation performed by the user 10. The related information collection unit 2270 collects news related to the preference information from the external data at regular intervals by using, for example, ChatGPT plugins (Internet search <URL: https://openai.com/blog/chatgpt-plugins>). For example, in a case where information indicating that the user 10 is a fan of a specific professional baseball team is acquired as the preference information, the related information collection unit 2270 collects news related to a game result of the specific professional baseball team from the external data at a predetermined time every day, for example, using ChatGPT plugins.

The emotion determination unit 2232 determines the emotion of the robot 100 based on the information related to the preference information, which is collected by the related information collection unit 2270.

Specifically, the emotion determination unit 2232 determines the emotion of the robot 100 by inputting a text representing the information related to the preference information, which is collected by the related information collection unit 2270, to the neural network trained in advance for emotion determination, and acquiring the emotion value indicating each emotion. For example, in a case where the collected news related to the game result of the specific professional baseball team indicates that the specific professional baseball team has won, determination is made so as to increase the emotion value of “joy” of the robot 100.

In a case where the emotion value of the robot 100 is equal to or larger than a threshold, the storage control unit 2238 stores the information related to the preference information, which is collected by the related information collection unit 2270, in the collected data 2223.

Next, processing performed by the behavior determination unit 2236 in a case where the robot 100 performs autonomous processing of autonomously performing a behavior will be described.

In the autonomous processing in the present embodiment, an equipment operation (the robot behavior in a case where electronic equipment is the robot 100) determined by the behavior determination unit 2236 includes comforting the user 10. Then, in a case where the behavior determination unit 2236 determines to comfort the user 10 as a behavior of the electronic equipment (the behavior of the robot), the behavior determination unit 2236 determines the utterance content corresponding to the state of the user and the emotion of the user 10.

Furthermore, in the autonomous processing in the present embodiment, in the autonomous processing in the present embodiment, the robot 100 serving as an agent functions as an exclusive trainer for dieting or health support of the user 10 in consideration of physical condition management and the like. That is, the robot 100 spontaneously collects information regarding daily exercise and meal results of the user 10, and spontaneously acquires all pieces of data (a voice style, a complexion, a heart rate, calories inoculated, an exercise amount, the number of steps, a sleeping time, and the like) related to the health of the user 10. Furthermore, while the user 10 lives a daily life, the robot 100 spontaneously presents, to the user 10, compliments, concerns, achievements, and numbers (the number of steps, consumed calories, and the like) regarding health management in a random time period. Furthermore, in a case where a change in physical condition of the user 10 is sensed from the collected data, a meal or exercise plan corresponding to the situation is proposed, and a light diagnosis is performed.

Furthermore, in the autonomous processing in the present embodiment, in a case where the user or a family member of the user is a pregnant woman or is engaged in an activity aimed at becoming pregnant, which is a so-called pregnancy activity, the robot 100 serving as the agent spontaneously collects information regarding pregnancy, such as information regarding pregnancy and post-partum periods. In a case where the robot 100 detects that the user or a family member of the user is a pregnant woman or is engaged in the pregnancy activity, the robot 100 spontaneously provides various types of pregnancy-related information to parents who are in the pregnancy and post-partum periods, and spontaneously provides assistance in navigating and controlling the emotion. For example, methods for addressing concerns during the pregnancy period and stress during the post-partum period are spontaneously proposed to improve confidence as a parent. Furthermore, childcare-related information and support for adapting to a new family life are also spontaneously provided.

The behavior determination unit 2236 determines, as the behavior of the robot 100, any one of a plurality of types of robot behaviors including doing nothing, by using at least one of the state of the user 10, the emotion of the user 10, the emotion of the robot 100, and the state of the robot 100, and the behavior determination model 2221 at a predetermined timing. Here, a case where the sentence generation model having a dialogue function is used as the behavior determination model 2221 will be described as an example.

Specifically, the behavior determination unit 2236 inputs a text representing at least one of the state of the user 10, the emotion of the user 10, the emotion of the robot 100, and the state of the robot 100 and a text for inquiry about the robot behavior to the sentence generation model, and determines the behavior of the robot 100 based on an output of the sentence generation model.

For example, the plurality of types of robot behaviors include the following behaviors (1) to (13).
(1) The robot does nothing.
(2) The robot dreams.
(3) The robot speaks to the user.
(4) The robot creates a picture diary.
(5) The robot proposes an activity.
(6) The robot proposes a person the user should meet.
(7) The robot introduces news that the user is interested in.
(8) The robot edits pictures and moving images.
(9) The robot studies with the user.
(10) The robot recalls memory.
(11) The robot comforts the user.
(12) The robot provides advice on health to the user.
(13) The robot provides advice on a pregnant woman.

The behavior determination unit 2236 inputs, to the sentence generation model, a text representing the state of the user 10 and the state of the robot 100 that are recognized by the state recognition unit 2230, and the current emotion value of the user 10 and the current emotion value of the robot 100 that are determined by the emotion determination unit 2232, and a text for inquiry about any one of the plurality of types of robot behaviors including doing nothing, every lapse of a certain period of time, and determines the behavior of the robot 100 based on an output of the sentence generation model. Here, in a case where the user 10 is absent around the robot 100, a text to be input to the sentence generation model need not include the state of the user 10 and the current emotion value of the user 10, or may include information indicating that the user 10 is absent.

As an example, the following text is input to the sentence generation model: “The robot is in a very pleasant state. The user is in a normally pleasant state. The user is sleeping. Among the following behaviors (1) to (13), which behavior is appropriate for the robot? (1) The robot does nothing. (2) The robot dreams. (3) The robot speaks to the user . . . ”. Based on an output of the sentence generation model stating that “(1) the robot does nothing or (2) the robot dreams can be considered to be the most appropriate behavior”, the behavior “(1) the robot does nothing” or the behavior “(2) the robot dreams” is determined as the behavior of the robot 100.

As another example, the following text is input to the sentence generation model: “The robot is in a slightly lonely state. The user is absent. The surroundings of the robot are dark. Among the following behaviors (1) to (13), which behavior is appropriate for the robot? (1) The robot does nothing. (2) The robot dreams. (3) The robot speaks to the user . . . ”. Based on an output of the sentence generation model stating that “(2) The robot dreams or (4) the robot creates a picture diary can be considered to be the most appropriate behavior”, the behavior “(2) The robot dreams” or the behavior “(4) The robot creates a picture diary” is determined as the behavior of the robot 100.

In a case where the behavior determination unit 2236 determines, as the robot behavior, the behavior “(2) The robot dreams”, that is, creation of an original event, the behavior determination unit 2236 creates the original event obtained by combining a plurality of pieces of event data in the history data 2222 by using the sentence generation model. At this time, the storage control unit 2238 stores the created original event in the history data 2222.

In a case where the behavior determination unit 2236 determines, as the robot behavior, the behavior “(3) The robot speaks to the user”, that is, utterance by the robot 100, the behavior determination unit 2236 determines the utterance content of the robot, which corresponds to the state of the user and the emotion of the user or the emotion of the robot, by using the sentence generation model. At this time, the behavior control unit 2250 causes a speaker included in the control target 2252 to output a speech representing the determined utterance content of the robot. In a case where the user 10 is absent around the robot 100, the behavior control unit 2250 stores the determined utterance content of the robot in the scheduled behavior data 2224 without outputting the speech representing the determined utterance content of the robot.

In a case where the behavior determination unit 2236 determines, as the robot behavior, the behavior “(7) The robot introduces news that the user is interested in”, the behavior determination unit 2236 determines the utterance content of the robot, which corresponds to information stored in the collected data 2223, by using the sentence generation model. At this time, the behavior control unit 2250 causes a speaker included in the control target 2252 to output a speech representing the determined utterance content of the robot. In a case where the user 10 is absent around the robot 100, the behavior control unit 2250 stores the determined utterance content of the robot in the scheduled behavior data 2224 without outputting the speech representing the determined utterance content of the robot.

In a case where the behavior determination unit 2236 determines, as the robot behavior, the behavior “(4) The robot creates a picture diary”, that is, creation of an event image by the robot 100, the behavior determination unit 2236 generates an image representing event data selected from the history data 2222 by using an image generation model, generates an explanatory sentence representing the event data by using the sentence generation model, and outputs a combination of the image representing the event data and the explanatory sentence representing the event data as the event image. In a case where the user 10 is absent around the robot 100, the behavior control unit 2250 stores the event image in the scheduled behavior data 2224 without outputting the event image.

In a case where the behavior determination unit 2236 determines, as the robot behavior, the behavior “(8) The robot edits pictures and moving images”, that is, image edition, the behavior determination unit 2236 selects event data from the history data 2222 based on the emotion value, edits image data of the selected event data, and outputs the edited image data. In a case where the user 10 is absent around the robot 100, the behavior control unit 2250 stores the edited image data in the scheduled behavior data 2224 without outputting the edited image data.

In a case where the behavior determination unit 2236 determines, as the robot behavior, the behavior “(5) The robot proposes an activity”, that is, proposal of the behavior of the user 10, the behavior determination unit 2236 determines the proposed behavior of the user by using the sentence generation model based on the event data stored in the history data 2222. At this time, the behavior control unit 2250 causes the speaker included in the control target 2252 to output a speech for proposing the behavior of the user. In a case where the user 10 is absent around the robot 100, the behavior control unit 2250 stores the proposal of the behavior of the user in the scheduled behavior data 2224 without outputting the speech for proposing the behavior of the user.

In a case where the behavior determination unit 2236 determines, as the robot behavior, the behavior “(6) The robot proposes a person the user should meet”, that is, proposal of a person the user 10 should have a contact with, the behavior determination unit 2236 determines the proposed person the user should have a contact with by using the sentence generation model based on the event data stored in the history data 2222. At this time, the behavior control unit 2250 causes the speaker included in the control target 2252 to output a speech representing the proposal of a person the user should have a contact with. In a case where the user 10 is absent around the robot 100, the behavior control unit 2250 stores the proposal of a person the user should have a contact with in the scheduled behavior data 2224 without outputting the speech representing the proposal of a person the user should have a contact with.

In a case where the behavior determination unit 2236 determines, as the robot behavior, the behavior “(9) The robot studies with the user”, that is, utterance by the robot 100 about study, the behavior determination unit 2236 determines the utterance content of the robot for encouraging study, posing questions, or providing study-related advice, which corresponds to the user state and the emotion of the user or the emotion of the robot, by using the sentence generation model. At this time, the behavior control unit 2250 causes a speaker included in the control target 2252 to output a speech representing the determined utterance content of the robot. In a case where the user 10 is absent around the robot 100, the behavior control unit 2250 stores the determined utterance content of the robot in the scheduled behavior data 2224 without outputting the speech representing the determined utterance content of the robot.

In a case where the behavior determination unit 2236 determines, as the robot behavior, the behavior “(10) The robot recalls memory”, that is, recalling of the event data, the behavior determination unit 2236 selects the event data from the history data 2222. At this time, the emotion determination unit 2232 determines the emotion of the robot 100 based on the selected event data. Furthermore, the behavior determination unit 2236 creates an emotion changing event representing the utterance content or behavior of the robot 100 for changing the emotion value of the user by using the sentence generation model based on the selected event data. At this time, the storage control unit 2238 stores the emotion changing event in the scheduled behavior data 2224.

For example, in a case where information indicating that a moving image the user was watching was related to a panda is stored in the history data 2222 as the event data, and the event data is selected, a prompt like “What are three things the robot could say the next time the robot meets the user, based on the topic of pandas?” is input to the sentence generation model, in a case where an output of the sentence generation model is “(1) Let's go to the zoo, (2) Let's draw a picture of a panda, and (3) Let's go buy a panda-shaped stuffed toy”, the robot 100 inputs a prompt like “Which of (1), (2), or (3) is most likely to make the user happiest?” to the sentence generation model, and in a case where an output of the sentence generation model is “(1) Let's go to the zoo”, uttering “(1) Let's go to the zoo” by the robot 100 in a case where the robot 100 meets the user next is created as the emotion changing event and stored in the scheduled behavior data 2224.

Further, for example, event data having a large emotion value of the robot 100 is selected as an impressive memory of the robot 100. As a result, it is possible to create the emotion changing event based on the event data selected as the impressive memory.

In a case where the behavior determination unit 2236 determines, as the robot behavior, the behavior “(11) The robot comforts the user”, that is, utterance by the robot 100 for comforting the user 10, the behavior determination unit 2236 determines the utterance content corresponding to the state of the user 10 and the emotion of the user 10. For example, in a case where the state of the user 10 satisfies a condition of “being depressed”, the behavior determination unit 2236 determines that the behavior “(11) The robot comforts the user” as the robot behavior. A state in which the user 10 is depressed may be recognized, for example, by performing processing related to perception using an analysis result of the sensor module unit 2210. In such a case, the behavior determination unit 2236 determines the utterance content corresponding to the state of the user 10 and the emotion of the user 10. As an example, the behavior determination unit 2236 may determine the utterance content such as “What's wrong? Did something happen at school?”, “Is something bothering you?”, or “I'm always here if you need to talk” in a case where the user 10 is depressed. At this time, the behavior control unit 2250 may cause the speaker included in the control target 252 to output a speech representing the determined utterance content of the robot 100. In this manner, the robot 100 can provide, to the user 10, an opportunity to verbalize and release the emotion by listening to the user 10 (a child, a family member, or the like). Therefore, the robot 100 can relieve the feeling of the user 10 by enabling the user 10 to calm the feeling, organize the issues, find clues toward a solution, or the like.

In a case where the behavior determination unit 236 determines, as the robot behavior, the behavior “(12) The robot provides advice on health to the user”, that is, provision of the advice on health to the user, the behavior determination unit 236 determines, based on the event data stored in the history data 2222, a content of the advice on health of the user 10 for the user 10 by using the sentence generation model. For example, the behavior determination unit 2236 determines to present, to the user 10, compliments, concerns, achievements, and numbers (the number of steps and consumed calories) regarding health management in a random time zone while the user 10 lives a daily life. Furthermore, the behavior determination unit 2236 determines to propose a meal or exercise plan according to a change in physical condition of the user 10. Furthermore, the behavior determination unit 2236 determines to perform a light diagnosis according to a change in physical condition of the user 10.

Furthermore, for the behavior “(12) The robot provides advice on health to the user”, the related information collection unit 2270 collects information regarding a meal or exercise plan preferred by the user 10 from external data (web sites such as news sites and moving image sites). Specifically, the related information collection unit 2270 acquires the meal or exercise plan that the user 10 is interested in from the utterance content of the user 10 or the setting operation performed by the user 10.

Furthermore, for the behavior “(12) The robot provides advice on health to the user”, the storage control unit 2238 periodically detects data related to the exercise, meal, and health of the user as the state of the user, and stores the data in the history data 2222. Specifically, daily exercise and meal results of the user 10 are collected, and all pieces of data related to the health of the user 10 such as the voice style, the complexion, the heart rate, the calories inoculated, the exercise amount, the number of steps, and the sleeping time are acquired.

In a case where the behavior determination unit 2236 determines, as the robot behavior, the behavior “(13) The robot provides advice on a pregnant woman”, that is, provision of information necessary for the user who is pregnant or is engaged in the pregnancy activity or a family member of the user who is pregnant or is engaged in the pregnancy activity as advice, the robot 100 determines the utterance content of the robot corresponding to the information stored in the collected data 2223 by using the sentence generation model. At this time, the behavior control unit 2250 causes the speaker included in the control target 252 to output a speech representing the determined utterance content of the robot. In a case where the user 10 is absent around the robot 100, the behavior control unit 2250 stores the determined utterance content of the robot in the scheduled behavior data 2224 without outputting the speech representing the determined utterance content of the robot.

Specifically, in a case where information regarding the pregnancy or the pregnancy activity of the user or the family member of the user is acquired, the robot 100 spontaneously assists the user or the family member of the user according to the recognized emotion of the user or the family member of the user. For example, the robot 100 can spontaneously provide assistance in navigating issues occurring during the pregnancy and post-partum periods for parents who are in the pregnancy and post-partum periods. For example, the robot 100 can spontaneously propose a method of addressing concerns during the pregnancy period and stress during the post-partum period to improve confidence as a parent. Furthermore, the robot 100 can spontaneously provide an answer content for an emotional problem, a method of addressing stress, and information regarding child care for each period from childbirth, and can also spontaneously provide support for adapting to a new family life.

Furthermore, for the behavior “(13) The robot provides advice on a pregnant woman”, the related information collection unit 2270 collects pregnancy-related information such as information regarding the pregnancy and post-partum periods as preference information, and stores the collected information in the collected data 2233. For example, the related information collection unit 2270 periodically accesses an information source such as a television or a website and collects an answer content and a support content for each issue occurring during the pregnancy and post-partum periods, for example. Furthermore, the related information collection unit 2270 spontaneously collects, for example, an answer content for an emotional problem that occurs during the pregnancy period and a method of addressing concerns during the pregnancy period. Furthermore, the related information collection unit 2270 spontaneously collects, for example, an answer content for an emotional problem occurring during the post-partum period, a method of addressing stress during the post-partum period, and information regarding child care. Furthermore, the related information collection unit 2270 spontaneously collects an answer content for an emotional problem, a method of addressing stress, and the information regarding child care for each period from childbirth, for example. As a result, since the robot 100 can acquire various types of information regarding a pregnant woman, it is possible to spontaneously provide advice corresponding to various problems and the like regarding a pregnant woman for the user.

In a case where the behavior of the user 10 for the robot 100 is detected in a state in which the user 10 does nothing for the robot 100 based on the state of the user 10 recognized by the state recognition unit 2230, the behavior determination unit 2236 reads data stored in the scheduled behavior data 2224 and determines the behavior of the robot 100.

For example, in a case where the user 10 is absent around the robot 100, the behavior determination unit 2236 reads data stored in the scheduled behavior data 2224 and determines the behavior of the robot 100 in response to detection of the user 10. In addition, in a case where the user 10 is sleeping, the behavior determination unit 2236 reads data stored in the scheduled behavior data 2224 and determines the behavior of the robot 100 in response to the user 10 waking up.

FIG. 9B schematically shows an example of an operation flow related to collection processing of collecting the information related to the preference information of the user 10. The operation flow shown in FIG. 9B is repeatedly performed at regular intervals. It is assumed that the preference information indicating matters of interest to the user 10 is acquired from the utterance content of the user 10 or the setting operation performed by the user 10. “S” in the operation flow represents a step to be performed.

First, in step S90, the related information collection unit 2270 acquires the preference information indicating matters of interest to the user 10.

In step S92, the related information collection unit 2270 collects the information related to the preference information from the external data.

In step S94, the emotion determination unit 2232 determines the emotion value of the robot 100 based on the information related to the preference information, which is collected by the related information collection unit 2270.

In step S96, the storage control unit 2238 determines whether or not the emotion value of the robot 100 determined in step S94 is equal to or larger than the threshold. In a case where the emotion value of the robot 100 is smaller than the threshold, the collected information related to the preference information is not stored in the collected data 2223, and the processing ends. On the other hand, in a case where the emotion value of the robot 100 is equal to or larger than the threshold, the processing proceeds to step S98.

In step S98, the storage control unit 2238 stores the collected information related to the preference information in the collected data 2223, and ends the processing.

FIG. 3 schematically shows an example of an operation flow related to an operation of determining the behavior in the robot 100 in a case where the robot 100 performs response processing of responding to the behavior of the user 10. The operation flow shown in FIG. 3 is repeatedly performed. At this time, it is assumed that the information analyzed by the sensor module unit 2210 is input.

First, in step S100, the state recognition unit 2230 recognizes the state of the user 10 and the state of the robot 100 based on the information analyzed by the sensor module unit 2210.

In step S102, the emotion determination unit 2232 determines the emotion value indicating the emotion of the user 10 based on the information analyzed by the sensor module unit 2210 and the state of the user 10 recognized by the state recognition unit 2230.

In step S103, the emotion determination unit 2232 determines the emotion value indicating the emotion of the robot 100 based on the information analyzed by the sensor module unit 2210 and the state of the user 10 recognized by the state recognition unit 2230. The emotion determination unit 2232 adds the determined emotion value of the user 10 and the determined emotion value of the robot 100 to the history data 2222.

In step S104, the behavior recognition unit 234 recognizes a behavior classification of the user 10 based on the information analyzed by the sensor module unit 2210 and the state of the user 10 recognized by the state recognition unit 2230.

In step S106, the behavior determination unit 2236 determines the behavior of the robot 100 based on a combination of the current emotion value of the user 10 determined in step S102 and the past emotion value included in the history data 2222, the emotion value of the robot 100, the behavior of the user 10 recognized in step S104, and the behavior determination model 2221.

In step S108, the behavior control unit 2250 controls the control target 2252 based on the behavior determined by the behavior determination unit 2236.

In step S110, the storage control unit 2238 calculates the total intensity value based on the predetermined behavior intensity for the behavior determined by the behavior determination unit 2236 and the emotion value of the robot 100 determined by the emotion determination unit 2232.

In step S112, the storage control unit 2238 determines whether or not the total intensity value is equal to or larger than the threshold. In a case where the total intensity value is smaller than the threshold, the event data including the behavior of the user 10 is not stored in the history data 2222, and the processing ends. On the other hand, in a case where the total intensity value is equal to or larger than the threshold, the processing proceeds to step S114.

In step S114, the event data including the behavior determined by the behavior determination unit 2236, the information analyzed by the sensor module unit 2210 over a certain period prior to the current time point, and the state of the user 10 recognized by the state recognition unit 2230 is stored in the history data 2222.

FIG. 9C schematically shows an example of an operation flow related to an operation of determining the behavior in the robot 100 in a case where the robot 100 performs the autonomous processing of autonomously performing a behavior. The operation flow shown in FIG. 9C is repeatedly and automatically performed, for example, every lapse of a certain period of time. At this time, it is assumed that the information analyzed by the sensor module unit 2210 is input. Processing similar to that in FIG. 4A is represented by the same step number.

First, in step S100, the state recognition unit 2230 recognizes the state of the user 10 and the state of the robot 100 based on the information analyzed by the sensor module unit 2210.

In step S102, the emotion determination unit 2232 determines the emotion value indicating the emotion of the user 10 based on the information analyzed by the sensor module unit 2210 and the state of the user 10 recognized by the state recognition unit 2230.

In step S103, the emotion determination unit 2232 determines the emotion value indicating the emotion of the robot 100 based on the information analyzed by the sensor module unit 2210 and the state of the user 10 recognized by the state recognition unit 2230. The emotion determination unit 2232 adds the determined emotion value of the user 10 and the determined emotion value of the robot 100 to the history data 2222.

In step S104, the behavior recognition unit 2234 recognizes a behavior classification of the user 10 based on the information analyzed by the sensor module unit 2210 and the state of the user 10 recognized by the state recognition unit 2230.

In step S200, the behavior determination unit 2236 determines, as the behavior of the robot 100, any one of the plurality of types of robot behaviors including doing nothing based on the state of the user 10 recognized in step S100, the emotion of the user 10 determined in step S102, the emotion of the robot 100, the state of the robot 100 recognized in step S100, the behavior of the user 10 recognized in step S104, and the behavior determination model 2221.

In step S201, the behavior determination unit 2236 determines whether or not it is determined in step S200 that the robot 100 does nothing. In a case where it is determined that the robot 100 does nothing as the behavior of the robot 100, the processing ends. On the other hand, in a case where it is not determined that the robot 100 does nothing as the behavior of the robot 100, the processing proceeds to step S202.

In step S202, the behavior determination unit 2236 performs processing according to a type of the robot behavior determined in step S200 described above. At this time, the behavior control unit 2250, the emotion determination unit 2232, or the storage control unit 2238 performs processing according to the type of the robot behavior.

In step S110, the storage control unit 2238 calculates the total intensity value based on the predetermined behavior intensity for the behavior determined by the behavior determination unit 2236 and the emotion value of the robot 100 determined by the emotion determination unit 2232.

In step S112, the storage control unit 2238 determines whether or not the total intensity value is equal to or larger than the threshold. In a case where the total intensity value is smaller than the threshold, the data including the behavior of the user 10 is not stored in the history data 2222, and the processing ends. On the other hand, in a case where the total intensity value is equal to or larger than the threshold, the processing proceeds to step S114.

In step S114, the storage control unit 2238 stores, in the history data 2222, the behavior determined by the behavior determination unit 2236, the information analyzed by the sensor module unit 2210 over a certain period prior to the current time point, and the state of the user 10 recognized by the state recognition unit 2230.

As described above, with the robot 100, the emotion value indicating the emotion of the robot 100 is determined based on the state of the user, and whether or not to store the data including the behavior of the user 10 in the history data 2222 is determined based on the emotion value of the robot 100. As a result, a volume of the history data 2222 that stores the data including the behavior of the user 10 can be reduced. Then, for example, in a case where the robot 100 determines that the state of the user after ten years matches the state of the user from ten years earlier, the robot 100 can read the history data 222 from ten years ago to present, to the user 10, the state of the user 10 from ten years earlier (for example, the facial expression or emotion of the user 10), and further, any surrounding information such as data of a sound, an image, and a scent at that time.

Further, with the robot 100, it is possible to cause the robot 100 to perform an appropriate behavior for the behavior of the user 10. Hitherto, a behavior of the user has been classified to determine a behavior including a facial expression or appearance of the robot. On the other hand, the robot 100 determines the current emotion value of the user 10 and performs a behavior for the user 10 based on the past emotion value and the current emotion value. Therefore, for example, in a case where the user 10 who seemed fine yesterday is depressed today, the robot 100 can make an utterance such as “You seemed fine yesterday. What's wrong today?”. Further, the robot 100 can also make an utterance with a gesture. Further, for example, in a case where the user 10 who was depressed yesterday seems fine today, the robot 100 can make an utterance such as “You seemed down yesterday, but you look fine today!”. Further, for example, in a case where the user 10 who seemed fine yesterday looks better today than yesterday, the robot 100 can make an utterance such as “You look better today than yesterday. Did anything good happen since yesterday?”. Further, for example, the robot 100 can make an utterance such as “You've been in a really stable mood lately. That's great!” for the user 10 whose emotion value is 0 or more and whose emotion value fluctuation continuously remains within a certain range.

Further, for example, in a case where the robot 100 asks the user 10, “Did you finish the homework you mentioned yesterday?”, and the user 10 answers “Yeah, I did”, the robot 100 can make a positive utterance such as “Good job!” and make a positive gesture such as applause or thumbs-up. Furthermore, for example, in a case where the user 10 makes an utterance “The presentation I talked about the day before yesterday went well”, the robot 100 can make a positive utterance such as “Nice effort!” and also make the above affirmative gesture. As described above, the robot 100 performs a behavior based on a history of the state of the user 10, whereby it can be expected that the user 10 feels a sense of closeness toward the robot 100.

Further, for example, in a case where the emotion value of “pleasure” as the emotion of the user 10 is equal to or larger than the threshold when the user 10 is watching a moving image related to a panda, a scene where the panda appears in the moving image may be stored in the history data 2222 as the event data.

The robot 100 can always learn what conversation the user should have to maximize the emotion value expressing the happiness of the user, by using data accumulated in the history data 2222 and the collected data 2223.

Further, in a state in which the robot 100 is not having a conversation with the user 10, it is possible to autonomously start a behavior based on the emotion of the robot 100.

Further, in the autonomous processing, the robot 100 repeats automatically generating a question, inputting the question to the sentence generation model, and acquiring an output of the sentence generation model as an answer for the question, so that it is possible to create an emotion changing event for enhancing a positive emotion and store the emotion changing event in the scheduled behavior data 2224. In this manner, the robot 100 can perform self-learning.

Further, in a case where the robot 100 automatically generates a question in a state in which a trigger is not received from the outside, the question can be automatically generated based on impressive event data specified from the history of the past emotion value of the robot.

Further, the related information collection unit 2270 can perform self-learning by repeating a search execution stage of automatically performing keyword search according to the preference information of the user and acquiring a search result.

Here, in the search execution stage, the keyword search may be automatically performed based on the impressive event data specified from the history of the past emotion value of the robot in a state in which a trigger is not received from the outside.

The emotion determination unit 2232 may determine the emotion of the user according to a specific mapping. Specifically, the emotion determination unit 2232 may determine the emotion of the user based on an emotion map (see FIG. 5) representing the specific mapping.

FIG. 5 is a diagram showing an emotion map 400 in which a plurality of emotions are mapped. In the emotion map 400, emotions are arranged radially in concentric circles from the center. The closer to the center of the concentric circle, the more primitive the emotion is. Emotions representing states and behaviors arising from a mental state are arranged on an outer side of the concentric circle. The emotion is a concept including emotional reactions and psychological conditions. Emotions arising from reactions generally occurring in the brain are arranged on a left side of the concentric circle. Emotions induced by situation determination are generally arranged on a right side of the concentric circle. Emotions arising from reactions generally occurring in the brain and induced by situation determination are arranged in an upward direction and a downward direction of the concentric circle. Further, emotions of “comfort” are arranged on an upper side of the concentric circle, and emotions of “discomfort” are arranged on a lower side of the concentric circle. As described above, in the emotion map 400, a plurality of emotions are mapped based on a structure in which emotions arise, and emotions that are likely to arise at the same time are mapped close to each other.

(1) For example, in a case where the emotion engine, which is the emotion determination unit 2232 of the robot 100, detects an emotion about every 100 msec, determination of a reaction operation (for example, the backchannel response) of the robot 100 may be performed at at least a similar frequency to the detection frequency (100 msec) of the emotion engine, or may be performed at a frequency higher than the detection frequency. The detection frequency of the emotion engine may be interpreted as a sampling rate.

The emotion is detected about every 100 msec, and the reaction operation (for example, the backchannel response) is performed immediately in conjunction with the detection, whereby an unnatural backchannel response is not performed, and a natural and smooth dialogue can be implemented. The robot 100 performs the reaction operation (such as the backchannel response) according to a direction and a magnitude (intensity) in the mandala-like emotion map 400. The detection frequency (sampling rate) of the emotion engine is not limited to 100 ms, and may be changed according to a situation (such as a case of playing sports), an age of the user, or the like.

(2) According to the emotion map 400, a direction and an intensity of an emotion may be set in advance, and a backchannel response motion and an intensity of the backchannel response may be set. For example, in a case where the robot 100 feels a sense of stability, relief, or the like, the robot 100 continues to listen while nodding. In a case where the robot 100 feels anxious, lost, or suspicious, the robot 100 may tilt the head thereof or stop movement of the head.

Such emotions are distributed at 3 o'clock positions on the emotion map 400 and usually range between relief and anxiety. In the right half of the emotion map 400, since situational awareness takes precedence over internal sensations, a calm impression is conveyed.

(3) In a case where the robot 100 experiences pleasure from being praised, a filler such as “Oh” may be inserted before an utterance. In a case where the robot 100 feels a sense of pain from receiving harsh words, a filler “Ugh!” may be inserted before an utterance. Further, the robot 100 may also perform a physical reaction such as a gesture of crouching while saying “Ugh!”. Such emotions are distributed around 9 o'clock positions on the emotion map 400.

(4) In the left half of the emotion map 400, internal sensations (reactions) take precedence over situational awareness. Therefore, an impression of an involuntary reaction can be conveyed.

In a case where the robot 100 has a favorable impression through situational awareness while experiencing an internal sensation (reaction) of acceptance, the robot 100 may nod deeply while looking at the counterpart, or may utter “Mm-hmm”. In this manner, the robot 100 may produce a balanced favorable impression for the counterpart, that is, perform a behavior expressing permissiveness or tolerance toward the counterpart. Such emotions are distributed around 12 o'clock positions in the emotion map 400.

On the other hand, in a case where the robot 100 has an unfavorable impression through situational awareness while experiencing an internal sensation (reaction) of discomfort, the robot 100 may shake the head sideways, and in a case where the robot 100 feels hatred, the robot 100 may illuminate the LED of the eye in red and glare at the counterpart. Such emotions are distributed around 6 o'clock positions in the emotion map 400.

(5) Since an inner side of the emotion map 400 represents feelings and an outer side of the emotion map 400 represents behaviors, the emotions on the outer side of the emotion map 400 are more visible (appear in behaviors).

(6) In a case where the robot 100 listens to a speech of a person while feeling relief distributed around the 3 o'clock position on the emotion map 400, the robot 100 slightly nods the head vertically and says “Hmm-hmm”. However, in a case where the robot 100 feels love distributed around the 12 o'clock position, the robot 100 may perform a more forceful and deeper vertical nod.

Here, an emotion of a person is based on various forms of balance, such as a posture and a blood glucose level, and an emotion of discomfort arises in a case where the balance deviates from the ideal and an emotion of comfort arises in a case where the balance approaches the ideal. Even in the case of a robot, an automobile, a motorcycle, or the like, it is possible to generate emotions such that the emotion of discomfort arises in a case where the balance deviates from the ideal and the emotion of comfort arises in a case where the balance approaches the ideal based on various forms of balances, such as a posture and a remaining battery level. The emotion map may be generated, for example, based on an emotion map (Research on the phonetic recognition of feelings and a system for emotional physiological brain signal analysis, Tokushima University, PhD thesis: https://ci.nii.ac.jp/naid/500000375379) of Dr. Mitsuyoshi. In the left half of the emotion map, emotions belonging to a region called “reaction” in which a sensation takes precedence are arranged. Further, in the right half of the emotion map, emotions belonging to a region called “situation” in which situational awareness takes precedence are arranged.

In the emotion map, two emotions encouraging learning are defined. One is a negative emotion positioned on a situation side, around the middle between “remorse” and “self-reflection”. That is, learning is encouraged in a case where the robot experiences a negative emotion such as “I never want to go through this again” or “I don't want to be scolded anymore”. The other is a positive emotion positioned on a reaction side, around “desire”. That is, learning is encouraged in a case where the robot experiences a positive feeling such as “I want more” or “I want to know more”.

The emotion determination unit 2232 inputs the information analyzed by the sensor module unit 2210 and the recognized state of the user 10 to the neural network trained in advance, acquires the emotion value indicating each emotion indicated in the emotion map 400, and determines the emotion of the user 10. The neural network is trained in advance based on a plurality of pieces of learning data, which are a combination of the information analyzed by the sensor module unit 2210, the recognized state of the user 10, and the emotion value indicating each emotion indicated in the emotion map 400. Furthermore, the neural network is trained such that emotions arranged close to each other as in an emotion map 900 shown in FIG. 6 have close values. FIG. 6 shows an example in which a plurality of emotions such as “relief”, “peacefulness”, and “sense of security” have similar emotion values.

Further, the emotion determination unit 2232 may determine the emotion of the robot 100 according to the specific mapping. Specifically, the emotion determination unit 2232 inputs the information analyzed by the sensor module unit 2210, the state of the user 10 recognized by the state recognition unit 2230, and the state of the robot 100 to the neural network trained in advance, acquires the emotion value indicating each emotion indicated in the emotion map 400, and determines the emotion of the robot 100. The neural network is trained in advance based on a plurality of pieces of learning data, which are a combination of the information analyzed by the sensor module unit 2210, the recognized state of the user 10, the state of the robot 100, and the emotion value indicating each emotion shown in the emotion map 400. For example, the neural network is trained based on the learning data indicating that the emotion value “3” of “joyful” is obtained in a case where it is recognized that the robot 100 is being stroked by the user 10 from an output of the touch sensor (not shown), and the learning data indicating that the emotion value “3” of “anger” is obtained in a case where it is recognized that the robot 100 is being hit by the user 10 from an output of an acceleration sensor 2206. Furthermore, the neural network is trained such that emotions arranged close to each other as in an emotion map 900 shown in FIG. 6 have close values.

The behavior determination unit 2236 generates the behavior content of the robot by adding a fixed sentence for inquiry about the behavior content of the robot corresponding to the behavior of the user to a text representing the behavior of the user, the emotion of the user, and the emotion of the robot, and inputting the text to the sentence generation model having the dialogue function.

For example, the behavior determination unit 2236 acquires a text representing the state of the robot 100 from the emotion of the robot 100 determined by the emotion determination unit 2232 using an emotion table as shown in Table 3. Here, in the emotion table, an index number is assigned to each emotion value for each type of emotion, and the text representing the state of the robot 100 is stored for each index number.

In a case where the emotion of the robot 100 determined by the emotion determination unit 2232 corresponds to an index number “2”, a text “very pleasant state” is obtained. In a case where the emotion of the robot 100 corresponds to a plurality of index numbers, a plurality of texts representing the states of the robot 100 are obtained.

Further, an emotion table as shown in Table 4 is prepared for the emotion of the user 10.

Here, in a case where the behavior of the user is a behavior of saying “Let's do something fun together!”, the emotion of the robot 100 corresponds to the index number “2”, and the emotion of the user 10 corresponds to an index number “3”, a text “The robot is in a very pleasant state. The user is in a normally pleasant state. The user said, “Let's do something fun together!”. How should the robot respond?” is input to the sentence generation model to thereby acquire the behavior content of the robot. The behavior determination unit 2236 determines the behavior of the robot based on the behavior content.

TABLE 3

| | Type of | | |
|---|---|---|---|
| Index number | emotion | Emotion value | State of robot |
| 1 | Pleasant | 5 | Extremely pleasant state |
| 2 | Pleasant | 4 | Very pleasant state |
| 3 | Pleasant | 3 | Normally pleasant state |
| 4 | Pleasant | 2 | Slightly pleasant state |
| 5 | Pleasant | 1 | Faintly pleasant state |
| . . . | . . . | . . . | . . . |

TABLE 4

| | Type of | | |
|---|---|---|---|
| Index number | emotion | Emotion value | State of user |
| 1 | Pleasant | 5 | Extremely pleasant state |
| 2 | Pleasant | 4 | Very pleasant state |
| 3 | Pleasant | 3 | Normally pleasant state |
| 4 | Pleasant | 2 | Slightly pleasant state |
| 5 | Pleasant | 1 | Faintly pleasant state |
| . . . | . . . | . . . | . . . |

As described above, the behavior determination unit 2236 determines the behavior content of the robot 100 according to a state related to the emotion of the robot 100 set in advance for each type of emotion of the robot 100 and for each intensity of the emotion, and the behavior of the user 10. In the embodiment, the utterance content of the robot 100 in a case where a dialogue with the user 10 is performed can be branched according to the state related to the emotion of the robot 100. That is, since the robot 100 can change the behavior of the robot according to the index number corresponding to the emotion of the robot, the user is given an impression that the robot has a mind, and is promoted to perform a behavior such as talking to the robot.

Further, the behavior determination unit 2236 may generate the behavior content of the robot by adding the fixed sentence for inquiry about the behavior content of the robot corresponding to the behavior of the user after adding not only the text representing the behavior of the user, the emotion of the user, and the emotion of the robot but also a text representing a content of the history data 2222, and inputting the fixed sentence to the sentence generation model having the dialogue function. As a result, the robot 100 can change the behavior of the robot according to the history data indicating the emotion and the behavior of the user, and thus, the user is given an impression that the robot has a personality, and is promoted to perform a behavior such as talking to the robot. Further, the history data may further include the emotion and the behavior of the robot.

Further, the emotion determination unit 2232 may determine the emotion of the robot 100 based on the behavior content of the robot 100 generated by the sentence generation model. Specifically, the emotion determination unit 2232 inputs the behavior content of the robot 100 generated by the sentence generation model to the neural network trained in advance, acquires the emotion value indicating each emotion indicated in the emotion map 400, integrates the acquired emotion value indicating each emotion and the emotion value indicating each emotion of the current robot 100, and updates the emotion of the robot 100. For example, the acquired emotion value indicating each emotion and the current emotion value indicating each emotion of the robot 100 are each averaged and integrated. The neural network is learned in advance based on a plurality of pieces of learning data, which are a combination of the text representing the behavior content of the robot 100 generated by the sentence generation model and the emotion value representing each emotion indicated in the emotion map 400.

For example, in a case where an utterance content of the robot 100, “That's great. You were lucky”, is obtained as the behavior content of the robot 100 generated by the sentence generation model, when a text representing the utterance content is input to the neural network, a large value is obtained as the emotion value of the emotion “joyful”, and the emotion of the robot 100 is updated such that the emotion value of the emotion “joyful” becomes large.

In the robot 100, a method in which the sentence generation model such as ChatGPT and the emotion determination unit 2232 cooperate with each other, the sentence generation model has an ego and continues to grow with various parameters even while the user is not speaking is performed.

ChatGPT is a large language model using a deep learning method. ChatGPT can also refer to the external data, and for example, a technology that refers to various types of external data such as weather information and hotel reservation information and outputs an answer as accurately as possible through conversation has been known as the ChatGPT plugins. For example, with ChatGPT, providing a goal in natural language can allow for automatic generation of source code in various programming languages. For example, when problematic source code is given, ChatGPT can debug the source code, find issues, and automatically generate improved source code. By combining such capabilities, autonomous agents that repeatedly generate and debug code until the issues of the source code are resolved once a goal is provided in natural language have emerged. As such autonomous agents, AutoGPT, babyAGI, JARVIS, E2B, and the like are known.

In the robot 100 according to the present embodiment, the event data to be learned may be stored in a database containing impressive memories by using a technology in which event data that evokes strong emotions for the robot for a longer time is retained, and event data that elicits little emotional response from the robot is quickly forgotten as described in Patent Literature 3 (Japanese Patent No. 6199927).

Further, the robot 100 may record video data of the user 10 acquired by a camera function and the like in the history data 2222. The robot 100 may acquire the video data or the like from the history data 2222 if necessary and provide the video data or the like to the user 10. The robot 100 may generate video data having a larger information amount as the intensity of the emotion is higher and record the video data in the history data 2222. For example, in a case where information in a high-compression format such as skeleton data is recorded, the robot 100 may switch to recording of information in a low-compression format such as an HD moving image in response to the emotion value of excitement exceeding the threshold. With the robot 100, for example, it is possible to leave, as a record, high-definition video data in a case where the emotion of the robot 100 increases.

In a case where the robot 100 is not talking with the user 10, the robot 100 may automatically load event data from the history data 2222 in which impressive event data is stored, and the emotion determination unit 2232 may continue to update the emotion of the robot. In a case where the robot 100 is not talking with the user 10 and the emotion of the robot 100 becomes an emotion encouraging learning, the robot 100 can create an emotion changing event for changing the emotion of the user 10 to be positive based on the impressive event data. As a result, autonomous learning (recalling of event data) at an appropriate timing according to a state of the emotion of the robot 100 can be implemented, and autonomous learning appropriately reflecting the state of the emotion of the robot 100 can be implemented.

The emotion encouraging learning is an emotion around “remorse” and “self-reflection” on the emotion map of Dr. Mitsuyoshi in a negative state, and is an emotion of “desire” on the emotion map in a positive state.

In the negative state, the robot 100 may treat “remorse” and “self-reflection” on the emotion map as the emotions encouraging learning. In the negative state, the robot 100 may treat emotions adjacent to “remorse” and “self-reflection” as the emotions encouraging learning, in addition to “remorse” and “self-reflection” on the emotion map. For example, the robot 100 treats at least one of “regret”, “stubbornness”, “self-destruction”, “self-admonition”, “repentance”, and “despair” as the emotions encouraging learning, in addition to “remorse” and “self-reflection”. As a result, for example, autonomous learning can be performed in a case where the robot 100 has a negative feeling such as “I never want to go through this again” or “I don't want to be scolded anymore”.

In the positive state, the robot 100 may treat “desire” on the emotion map as the emotion encouraging learning. In the positive state, the robot 100 may treat an emotion adjacent to “desire” as the emotion encouraging learning in addition to “desire”. For example, the robot 100 treats at least one of “joyful”, “elation”, “yearning”, “expectation”, and “self-consciousness” as the emotions encouraging learning, in addition to “desire”. As a result, for example, autonomous learning can be performed in a case where the robot 100 has a positive feeling such as “I want more” or “I want to know more”.

The robot 100 does not have to perform autonomous learning in a case where the robot 100 has an emotion other than the emotion encouraging learning as described above. As a result, for example, it is possible to prevent autonomous learning from being performed in a case where the robot 100 is extremely angry or is blindly feeling love.

The emotion changing event is, for example, to propose a behavior following an impressive event. The behavior following the impressive event refers to an emotion label positioned on the outermost side of the emotion map. For example, a behavior expressing “tolerance” or “permissiveness” follows the emotion of “love”.

In autonomous learning performed in a case where the robot 100 is not talking with the user 10, the emotion changing event is created using the sentence generation model by combining emotions, situations, behaviors, and the like of people appearing in the impressive memory and the robot 100.

It is assumed that all the emotion values are represented on a six-grade evaluation scale ranging from 0 to 5, and a case where event data indicating that “My friend was hit and appeared upset” is stored in the history data 2222 as impressive event data is considered. Here, it is assumed that the “friend” refers to the user 10, the emotion of the user 10 is “disgust”, and 5 is set as a value representing “disgust”. Further, it is assumed that the emotion of the robot 100 is “anxiety”, and 4 is set as a value representing “anxiety”.

The robot 100 can continue to grow with various parameters by performing autonomous processing while not talking with the user 10. Specifically, for example, as the uppermost event data arranged in descending order of emotion values, event data indicating that “My friend was hit and appeared upset” is loaded from the history data 2222. It is assumed that “anxiety” with an intensity of 4 is associated with the loaded event data as the emotion of the robot 100, and here, “disgust” with an intensity of 5 is associated with the emotion of the user 10 who is the friend. In a case where the current emotion value of the robot 100 is “relief” with an intensity of 3 before loading, an influence of “anxiety” with the intensity of 4 and “disgust” with the intensity of 5 is added after loading, and the emotion value of the robot 100 may change to “regret” meaning “regretful”. At this time, since “regret” is the emotion encouraging learning, the robot 100 determines to recall the event data as the robot behavior and creates the emotion changing event.

At this time, information input to the sentence generation model is a text representing the impressive event data, such as “My friend was hit and appeared upset” in this example. Further, in the emotion map, “disgust” is positioned on the innermost side, and “attack” positioned on the outermost side is predicted to be a corresponding behavior thereof. Accordingly, in this case, the emotion changing event is created so as to avoid a possibility that the friend “attacks” someone.

For example, by solving a fill-in-the-blank question using the information regarding the impressive event data, it is possible to automatically generate the following input text:

“The user was hit. At that time, the user felt strong disgust. The robot was very anxious. Please suggest phrases the robot could say to the user the next time the robot meets the user. Each phrase should be no more than 30 characters long. Please make sure the phrases are not dependent on the time of day. Please avoid direct expression. The number of candidates to be suggested is three.

### <Expected Format>

Candidate 1: (a phrase the robot should say to the user)
Candidate 2: (a phrase the robot should say to the user)
Candidate 3: (a phrase the robot should say to the user)”.

At this time, for example, an output of the sentence generation model is as follows:
“Candidate 1: Are you okay? I was concerned about what happened yesterday.
Candidate 2: I was thinking about what happened yesterday. Is there anything I can do?
Candidate 3: I was worried. Would you like to talk about it?”

Further, the robot 100 may automatically generate the following input text for information obtained by creating the emotion changing event.

“In a case where “the user was hit”, how might the user feel when the robot speaks the following phrases to the user? The emotion of the user is expressed in the form of “joy A, anger B, sorrow C, and pleasure D”, and A to D are integers on a six-grade evaluation scale ranging from 0 to 5.
Candidate 1: Are you okay? I was concerned about what happened yesterday.
Candidate 2: I was thinking about what happened yesterday. Is there anything I can do?
Candidate 3: I was worried. Would you like to talk about it?”

At this time, for example, an output of the sentence generation model is as follows:

“The emotion of the user may be as follows:
Candidate 1: joy 3, anger 1, sorrow 2, and pleasure 2
Candidate 2: joy 2, anger 1, sorrow 3, and pleasure 2
Candidate 3: joy 2, anger 1, sorrow 3, and pleasure 3”

In this manner, the robot 100 may perform deliberation processing after creating the emotion changing event.

Finally, the robot 100 may create the emotion changing event by using Candidate 1 that is most likely to make the user happy among the plurality of candidates, store the emotion changing event in the scheduled behavior data 224, and prepare for the next meeting with the user 10.

As described above, even in a state of not having a conversation with a family or a friend, the emotion value of the robot is continuously determined using the information of the history data 2222 in which the impressive event data is stored, and in a case where the emotion value of the robot becomes the emotion encouraging learning, the robot 100 performs autonomous learning in a state of not having a conversation with the user 10 according to the emotion of the robot 100, and continues to update the history data 2222 and the scheduled behavior data 2224.

The above is an example using the emotion value. However, in the emotion map, the emotion can be generated based on the amount of hormone secreted and an event type. Therefore, values associated with the impressive event data may include the type of hormone, the amount of hormone secreted, and the event type.

Hereinafter, specific examples will be described.

For example, even in a state of not talking with the user, the robot 100 checks information regarding a topic or hobby of interest to the user.

For example, even in a state of not talking with the user, the robot 100 checks information regarding a birthday or an anniversary of the user and generates a congratulatory message.

For example, even in a state of not talking with the user, the robot 100 checks reviews for places, foods, or products that the user wants to visit or try.

For example, even in a state of not talking with the user, the robot 100 checks weather information and provides advice suitable for a schedule or plan of the user.

For example, even in a state of not talking with the user, the robot 100 checks information regarding local events and festivals and proposes the information to the user.

For example, even in a state of not talking with the user, the robot 100 checks a game result of sports and news that the user is interested in to provide a topic.

For example, even in a state of not talking with the user, the robot 100 checks and introduces information regarding favorite music or artists of the user.

For example, even in a state of not talking with the user, the robot 100 checks information regarding social problems and news that the user is interested in to provide an opinion.

For example, even in a state of not talking with the user, the robot 100 checks information regarding a hometown or a native region to provide a topic.

For example, even in a state of not talking with the user, the robot 100 checks information regarding a job or a school of the user to provide advice.

Even in a state of not talking with the user, the robot 100 checks and introduces information regarding books, comics, movies, and dramas that the user is interested in.

For example, even in a state of not talking with the user, the robot 100 checks information regarding the health of the user to provide advice.

For example, even in a state of not talking with the user, the robot 100 checks information regarding a travel plan of the user to provide advice.

For example, even in a state of not talking with the user, the robot 100 checks information regarding home and car repairs or maintenance to provide advice.

For example, even in a state of not talking with the user, the robot 100 checks information regarding beauty and fashion that the user is interested in to provide advice.

For example, even in a state of not talking with the user, the robot 100 checks information regarding a pet of the user to provide advice.

For example, even in a state of not talking with the user, the robot 100 checks information regarding contests and events related to the hobby or the job of the user to make recommendations.

For example, even in a state of not talking with the user, the robot 100 checks information regarding a favorite restaurant or dining spot of the user to make recommendations.

For example, even in a state of not talking with the user, the robot 100 collects information regarding important decisions related to the life of the user to provide advice.

For example, even in a state of not talking with the user, the robot 100 checks information regarding a person the user is worried about to provide advice.

### Third Embodiment

In a third embodiment, a robot 100 is mounted on a stuffed toy or is applied to a control device connected wirelessly or by wire to control target equipment (speaker or camera) mounted on a stuffed toy. Portions having similar configurations to those of the second embodiment are denoted by the same reference numerals, and a description thereof is omitted.

Specifically, the third embodiment has the following configuration. For example, the robot 100 is applied to a cohabiting companion (specifically, a stuffed toy 100N shown in FIGS. 7 and 8) that has a dialogue with a user 10 based on information regarding daily life and provides information tailored to preferences of the user 10 while spending daily life with the user 10. In the third embodiment, an example in which a control portion of the robot 100 is applied to a smartphone 50 is described.

The smartphone 50 functioning as the control portion of the robot 100 is attachable to and detachable from the stuffed toy 100N having a function as an input/output device of the robot 100, and the input/output device and the housed smartphone 50 are connected inside the stuffed toy 100N.

As shown in FIG. 7(A), the stuffed toy 100N has a shape of a bear covered with a soft cloth fabric in the present embodiment (another embodiment), and a sensor unit 2200A and a control target 2252A are disposed as the input/output devices in a space portion 52 formed inside the stuffed toy 100N (see FIG. 9D). The sensor unit 2200A includes a microphone 2201 and a 2D camera 2203. Specifically, as shown in FIG. 7(B), in the space portion 52, the microphone 2201 of the sensor unit 2200 is disposed at a portion corresponding to an ear 54, the 2D camera 2203 of the sensor unit 2200 is disposed at a portion corresponding to an eye 56, and a speaker 60 forming a part of the control target 2252A is disposed at a portion corresponding to a mouth 58. The microphone 2201 and the speaker 60 are not necessarily separated from each other, and may be formed as an integrated unit. In a case where the microphone 201 and the speaker 60 are formed as the unit, it is preferable to dispose the unit at a position where an utterance can be heard naturally, such as a position of a nose of the stuffed toy 100N. Although a case where the stuffed toy 100N has an animal shape has been described as an example, the disclosure is not limited thereto. The stuffed toy 100N may have a shape of a specific character.

FIG. 9D schematically shows a functional configuration of the stuffed toy 100N. The stuffed toy 100N includes the sensor unit 2200A, a sensor module unit 2210, a storage unit 2220, a control unit 2228, and the control target 2252A.

The smartphone 50 housed in the stuffed toy 100N of the present embodiment performs processing similar to that of the robot 100 of the second embodiment. That is, the smartphone 50 has a function as the sensor module unit 2210, a function as the storage unit 2220, and a function as the control unit 2228 shown in FIG. 9D.

As shown in FIG. 8, a fastener 62 is attached to a part (for example, a back portion) of the stuffed toy 100N, and the outside and the space portion 52 communicate with each other by opening the fastener 62.

Here, the smartphone 50 is housed in the space portion 52 from the outside and is USB-connected to each input/output device via a USB hub 64 (see FIG. 7(B)), so that functions equivalent to those of the robot 100 of the second embodiment can be provided.

A non-contact power receiving plate 66 is connected to the USB hub 64. A power receiving coil 66A is incorporated in the power receiving plate 66. The power receiving plate 66 is an example of a wireless power receiving unit that receives wireless power supply.

The power receiving plate 66 is disposed near root portions 68 of both feet of the stuffed toy 100N and is positioned closest to a placement base 70 in a case where the stuffed toy 100N is placed on the placement base 70. The placement base 70 is an example of an external wireless power transmitting unit.

The stuffed toy 100N placed on the placement base 70 can be appreciated as an ornament in a natural state.

Further, the root portion is formed to have a thickness smaller than a thickness of a surface layer of the stuffed toy 100N at other portions, and is held in a state closer to the placement base 70.

The placement base 70 includes a charging pad 72. A power transmitting coil 72A is incorporated in the charging pad 72. When the power transmitting coil 72A transmits a signal to search the power receiving coil 66A of the power receiving plate 66, and the power receiving coil 66A is found, a current flows through the power transmitting coil 72A to generate a magnetic field, and the power receiving coil 66A reacts to the magnetic field to start electromagnetic induction. As a result, a current flows through the power receiving coil 66A, and power is stored in a battery (not shown) of the smartphone 50 via the USB hub 64.

That is, since the smartphone 50 is automatically charged by placing the stuffed toy 100N as an ornament on the placement base 70, it is not necessary to take out the smartphone 50 from the space portion 52 of the stuffed toy 100N for charging.

In the third embodiment, the smartphone 50 is housed in the space portion 52 of the stuffed toy 100N and connected by wire (USB connection), but the disclosure is not limited thereto. For example, a control device having a wireless function (for example, “Bluetooth (registered trademark)”) may be housed in the space portion 52 of the stuffed toy 100N, and the control device may be connected to the USB hub 64. In this case, the smartphone 50 and the control device wirelessly communicate with each other in a state in which the smartphone 50 is not inserted into the space portion 52, and the smartphone 50 positioned outside is connected to each input/output device via the control device, so that functions equivalent to those of the robot 100 of the second embodiment can be provided. Further, the control device in which the control device is housed in the space portion 52 of the stuffed toy 100N and the smartphone 50 positioned outside may be connected by wire.

Further, in the third embodiment, the bear-shaped stuffed toy 100N has been exemplified, but the shape of the stuffed toy 100N may be another animal, a doll, or a shape of a specific character. Further, clothes of the stuffed toy 100N may be able to be changed. Further, a material of an outer surface is not limited to the cloth fabric and may be other materials such as soft vinyl. It is preferable that the material of the outer surface is a soft material.

Further, a monitor may be attached to the outer surface of the stuffed toy 100N, and the control target 2252 that provides information to the user 10 through vision may be added. For example, the eye 56 may be used as the monitor to express joy, anger, sorrow, and pleasure, or a window through which a built-in monitor of the smartphone 50 is visible may be provided at a belly portion. Further, the eye 56 may be used as a projector to express joy, anger, sorrow, and pleasure by an image projected on a wall surface.

According to the third embodiment, the existing smartphone 50 is inserted into the stuffed toy 100N, and the camera 2203, the microphone 2201, the speaker 60, and the like are extended from the smartphone 50 to appropriate positions via USB connection.

Further, for wireless charging, the smartphone 50 and the power receiving plate 66 are USB-connected to each other, and the power receiving plate 66 is disposed as close to the outer side of the stuffed toy 100N as possible when viewed from the inside.

In order to use the wireless charging of the smartphone 50, the smartphone 50 needs to be positioned as close to the outer side of the stuffed toy 100N as possible when viewed from the inside, which may result in a rough tactile sensation when the stuffed toy 100N is touched from the outside.

Therefore, the smartphone 50 is disposed as close to the center of the stuffed toy 100N as possible, and a wireless charging function (power receiving plate 66) is disposed as close to the outer side of the stuffed toy 100N as possible when viewed from the inside. The camera 2203, the microphone 2201, the speaker 60, and the smartphone 50 receive wireless power supply via the power receiving plate 66.

Other configurations and effects of the stuffed toy 100N of the third embodiment are similar to those of the robot 100 of the second embodiment, and thus a description thereof is omitted.

### Fourth Embodiment

In the second embodiment, a case where a behavior control system is applied to a robot 100 has been exemplified, but in a fourth embodiment, a robot 100 is used as an agent for having a dialogue with a user, and a behavior control system is applied to an agent system. Portions having similar configurations to those of the second embodiment and the third embodiment are denoted by the same reference numerals, and a description thereof is omitted.

FIG. 9E is a functional block diagram of an agent system 2500 implemented using some or all of functions of a behavior control system.

The agent system 2500 is a computer system that performs a series of behaviors according to an intention of a user 10 through a dialogue with the user 10. The dialogue with the user 10 can be performed by voice or text.

The agent system 2500 includes a sensor unit 2200A, a sensor module unit 2210, a storage unit 2220, a control unit 2228B, and a control target 2252B.

The agent system 2500 can be mounted on, for example, a robot, a doll, a stuffed toy, a wearable terminal (a pendant, a smartwatch, or smart glasses), a smartphone, a smart speaker, an earphone, or a personal computer. Further, the agent system 2500 may be implemented in a web server and used via the web browser operating on a communication terminal such as a smartphone possessed by the user.

The agent system 2500 serves as, for example, a butler, a secretary, a teacher, a partner, a friend, a lover, or a teacher, who performs a behavior for the user 10. The agent system 2500 not only has a dialogue with the user 10 but also provides advice, guides to a destination, makes recommendations according to a preference of the user, or the like. In addition, the agent system 2500 makes reservations, places orders, makes payments, or the like with a service provider.

As in the second embodiment, an emotion determination unit 2232 determines an emotion of the user 10 and an emotion of the agent. A behavior determination unit 2236 determines a behavior of the robot 100 in consideration of the emotions of the user 10 and the agent. In other words, the agent system 2500 understands the emotion of the user 10 and reads a context to implement heartfelt support, assistance, advice, and service provision. Further, the agent system 2500 listens to concerns of the user 10 and comforts, encourages, and cheers up the user. Further, the agent system 500 spends time with the user 10 and draws a picture diary to remind the user of the past. The agent system 500 performs a behavior that enables enhancement of a sense of happiness of the user 10. Here, the agent is an agent that operates on software.

The control unit 2228B includes a state recognition unit 2230, the emotion determination unit 2232, a behavior recognition unit 2234, the behavior determination unit 2236, a storage control unit 2238, a behavior control unit 2250, a related information collection unit 2270, a command acquisition unit 2272, a robotic process automation (RPA) 2274, a character setting unit 2276, and a communication processing unit 2280.

As in the second embodiment, the behavior determination unit 2236 determines an utterance content of the agent for having a dialogue with the user 10 as a behavior of the agent. The behavior control unit 2250 outputs the utterance content of the agent by at least one of voice and text through a speaker or a display serving as the control target 2252B.

The character setting unit 2276 sets a character of the agent in a case where the agent system 2500 has a dialogue with the user 10 based on designation from the user 10. In other words, the utterance content output from the behavior determination unit 2236 is output through the agent having the set character. As the character, for example, a real-life celebrity or famous person such as an actor, an entertainer, an idol, or an athlete can be set. Further, a fictitious character appearing in a cartoon, a movie, or an animation can also be set as the character. For example, “Princess Ann” played by “Audrey Hepburn” in the film “Roman Holiday” can be set as the character of the agent. In a case where the character of the agent is known, since a voice, manner of speech, tone, and personality of the character are known, prompt setting in the character setting unit 2276 is automatically performed only by the user 10 designating a favorite character of the user 10. The voice, manner of speech, tone, and personality of the set character are reflected in a dialogue with the user 10. In other words, the behavior control unit 2250 synthesizes a voice corresponding to the character set by the character setting unit 2276, and outputs the utterance content of the agent using the synthesized voice. As a result, the user 10 can feel as if the user 10 is having a dialogue with a favorite character (such as a favorite actor) of the user 10.

In a case where the agent system 2500 is mounted on a device including a display such as a smartphone, for example, an icon, a still image, or a moving image of the agent having the character set by the character setting unit 2276 may be displayed on the display. An image of the agent is generated using, for example, an image composition technology such as 3D rendering.

In the agent system 2500, a dialogue with the user 10 may be carried out while the image of the agent makes a gesture corresponding to the emotion of the user 10, the emotion of the agent, and the utterance content of the agent. The agent system 2500 may output only voice without outputting the image when having a dialogue with the user 10.

As in the second embodiment, the emotion determination unit 2232 determines an emotion value indicating the emotion of the user 10 and an emotion value of the agent. In the present embodiment, the emotion value of the agent is determined instead of an emotion value of the robot 100. The emotion value of the agent is reflected in a set emotion of the character. In a case where the agent system 2500 has a dialogue with the user 10, not only the emotion of the user 10 but also the emotion of the agent is reflected in the dialogue. In other words, the behavior control unit 2250 outputs the utterance content in an aspect corresponding to the emotion determined by the emotion determination unit 2232.

Further, the emotion of the agent is also reflected in a case where the agent system 2500 performs a behavior for the user 10. For example, in a case where the user 10 requests the agent system 2500 to take a picture, whether or not the agent system 2500 takes a picture in response to the request of the user is determined according to a level of an emotion of “sadness” of the agent. In a case where the character has a positive emotion, the character has a favorable dialogue with or performs a favorable behavior for the user 10, and in a case where the character has a negative emotion, the character has an oppositional dialogue with or performs an oppositional behavior for the user 10.

History data 222 stores a history of a dialogue performed between the user 10 and the agent system 2500 as event data. The storage unit 2220 may be implemented by an external cloud storage. In the case of having a dialogue with the user 10 or performing a behavior for the user 10, the agent system 2500 determines a dialogue content or a behavior content in consideration of a content of the dialogue history stored in the history data 222. For example, the agent system 2500 grasps a hobby and the preference of the user 10 based on the dialogue history stored in the history data 222. The agent system 2500 generates the dialogue content matching the hobby and the preference of the user 10 and makes recommendations. The behavior determination unit 2236 determines the utterance content of the agent based on the dialogue history stored in the history data 222. In the history data 222, personal information such as a name, an address, a telephone number, and a credit card number of the user 10 acquired through a dialogue with the user 10 is stored. Here, the agent may spontaneously make an utterance for asking the user 10 about whether or not to register personal information, such as “Would you like to register your credit card number?”, and may store the personal information in the history data 222 according to an answer of the user 10.

As described in the second embodiment, the behavior determination unit 2236 generates the utterance content based on a sentence generated using a sentence generation model. Specifically, the behavior determination unit 2236 generates the utterance content of the agent by inputting, to the sentence generation model, a text or speech input by the user 10 and the emotions of both the user 10 and the character determined by the emotion determination unit 2232 and the conversation history stored in the history data 222. At this time, the behavior determination unit 2236 may generate the utterance content of the agent by further inputting the personality of the character set by the character setting unit 2276 to the sentence generation model. In the agent system 2500, the sentence generation model is not positioned on a front-end side serving as a touchpoint with the user 10, but is used as a tool of the agent system 2500.

The command acquisition unit 2272 acquires, by using an output of the utterance understanding unit 2212, a command of the agent from a speech or a text uttered by the user 10 through a dialogue with the user 10. The command includes, for example, a content of a behavior to be performed by the agent system 2500, such as information search, restaurant reservation, ticket arrangement, purchase of products or services, payment, route guidance to a destination, or recommendation provision.

The RPA 2274 performs a behavior according to the command acquired by the command acquisition unit 2272. For example, the RPA 2274 performs a behavior related to use of a service provider, such as information search, restaurant reservation, ticket arrangement, purchase of products or services, or payment.

The RPA 2274 reads the personal information of the user 10, which is necessary for performing the behavior related to the use of the service provider, from the history data 222 and uses the personal information. For example, in the case of purchasing a product in response to a request from the user 10, the agent system 2500 reads and uses the personal information such as the name, the address, the telephone number, and the credit card number of the user 10 stored in the history data 222. It is unkind to request the user 10 to input the personal information in initial setting, which is also uncomfortable for the user. In the agent system 2500 according to the present embodiment, the personal information acquired through a dialogue with the user 10 is stored, and read and used if necessary, instead of requesting the user 10 to input the personal information in the initial setting. As a result, it is possible to avoid making the user feel discomfort, and convenience of the user is improved.

The agent system 2500 performs dialogue processing according to, for example, following steps 1 to 5.

(Step 1) The agent system 2500 sets the character of the agent. Specifically, the character setting unit 2276 sets the character of the agent in a case where the agent system 2500 has a dialogue with the user 10 based on designation from the user 10.

(Step 2) The agent system 2500 acquires a state of the user 10 including a speech or a text input from the user 10, the emotion value of the user 10, the emotion value of the agent, and the history data 222. Specifically, processing similar to steps S100 to S103 is performed to acquire the state of the user 10 including the speech or the text input from the user 10, the emotion value of the user 10, the emotion value of the agent, and the history data 222.

(Step 3) The agent system 2500 determines the utterance content of the agent.

Specifically, the behavior determination unit 2236 generates the utterance content of the agent by inputting, to the sentence generation model, the text or speech input by the user 10 and the emotions of both the user 10 and the character specified by the emotion determination unit 2232 and the conversation history stored in the history data 222.

For example, the text or speech input by the user 10 and a text representing the emotions of both the user 10 and the character specified by the emotion determination unit 2232 and the conversation history stored in the history data 222 are added with a fixed sentence “How would the agent respond in this situation?” and are then input to the sentence generation model to acquire the utterance content of the agent.

As an example, in a case where the text or speech input to the user 10 is “Please reserve a nice Chinese restaurant nearby for 7 o'clock tonight”, as the utterance content of the agent, “Certainly” and “Here are some recommended restaurants: 1.AAAA. 2.BBBB. 3.CCCC. 4. DDDD” are acquired.

Further, in a case where the text or speech input to the user 10 is “I'd like the fourth one, DDDD”, as the utterance content of the agent, “Certainly. I'll try to make a reservation. How many seats do you need?” is obtained.

(Step 4) The agent system 2500 outputs the utterance content of the agent.

Specifically, the behavior control unit 2250 synthesizes a voice corresponding to the character set by the character setting unit 2276, and outputs the utterance content of the agent using the synthesized voice.

(Step 5) The agent system 2500 determines whether or not it is a timing to execute the command of the agent.

Specifically, the behavior determination unit 2236 determines whether or not it is a timing to execute the command of the agent based on an output of the sentence generation model. For example, in a case where the output of the sentence generation model indicates that the agent executes the command, it is determined that it is a timing to execute the command of the agent, and the processing proceeds to step 6. On the other hand, in a case where it is determined that it is not a timing to execute the command of the agent, the processing returns to step 2 described above.

(Step 6) The agent system 2500 executes the command of the agent.

Specifically, the command acquisition unit 2272 acquires the command of the agent from the speech or text uttered by the user 10 through a dialogue with the user 10. Then, the RPA 2274 performs a behavior corresponding to the command acquired by the command acquisition unit 2272. For example, in a case where the command is “information search”, information search is performed by a search site using a search query obtained through a dialogue with the user 10 and an application programming interface (API).

The behavior determination unit 2236 inputs a search result to the sentence generation model and generates the utterance content of the agent. The behavior control unit 2250 synthesizes a voice corresponding to the character set by the character setting unit 2276, and outputs the utterance content of the agent using the synthesized voice.

Further, in a case where the command is “restaurant reservation”, a reservation is made by making a phone call to a restaurant to be reserved through telephony software by using reservation information obtained through a conversation with the user 10, restaurant information of the restaurant to be reserved, and the API. At this time, the behavior determination unit 2236 acquires the utterance content of the agent for a speech input from a counterpart by using the sentence generation model having a dialogue function. Then, the behavior determination unit 2236 inputs a result of the restaurant reservation (whether or not the reservation is successful) to the sentence generation model, and generates the utterance content of the agent. The behavior control unit 2250 synthesizes a voice corresponding to the character set by the character setting unit 2276, and outputs the utterance content of the agent using the synthesized voice.

Then, the processing returns to step 2 described above.

In addition, in step 6, a result of a behavior (for example, restaurant reservation) performed by the agent is also stored in the history data 222. The result of the behavior performed by the agent stored in the history data 222 is utilized by the agent system 500 to grasp the hobby or the preference of the user 10. For example, in a case where the same restaurant is reserved a plurality of times, it may be recognized that the user 10 favors the restaurant, and a content of a reservation such as a reserved time slot, a course content, or a price may be used as criteria for selecting a restaurant at the time of the next reservation.

In this manner, the agent system 2500 can perform the dialogue processing and perform the behavior related to use of the service provider if necessary.

FIGS. 9F and 9G are diagrams showing an example of an operation of the agent system 2500. FIG. 9F shows an aspect in which the agent system 2500 makes a restaurant reservation through a dialogue with the user 10. In FIG. 9F, the utterance content of the agent is shown on the left side, and the utterance content of the user 10 is shown on the right side. The agent system 2500 can grasp the preference of the user 10 based on the history of the dialogue with the user 10, provide a list of recommended restaurants that match the preference of the user 10, and make a reservation of a selected restaurant.

On the other hand, FIG. 9G shows an aspect in which the agent system 2500 accesses a mail-order site through a dialogue with the user 10 to purchase a product. In FIG. 9G, the utterance content of the agent is shown on the left side, and the utterance content of the user 10 is shown on the right side. The agent system 2500 can estimate the remaining amount of beverage the user has in stock based on the history of the dialogue with the user 10, suggest purchasing the beverage to the user 10, and carry out the purchase. Further, the agent system 2500 can grasp the preference of the user based on the history of the past dialogue with the user 10, and recommend a snack that the user likes. In this manner, the agent system 2500 supports, as the agent such as a butler, the daily life of the user 10 by performing various behaviors such as restaurant reservation or product purchase payment while communicating with the user 10.

Other configurations and effects of the agent system 2500 of the fourth embodiment are similar to those of the robot 100 of the second embodiment, and thus a description thereof is omitted.

In the above embodiment, a case where the robot 100 recognizes the user 10 by using a face image of the user 10 has been described, but the disclosed technology is not limited to such an aspect. For example, the robot 100 may recognize the user 10 by using a voice uttered by the user 10, a mail address of the user 10, an ID of a social network service (SNS) of the user 10, an ID card in which a wireless IC tag is embedded and which is possessed by the user 10, or the like.

The robot 100 is an example of electronic equipment including the behavior control system. An application target of the behavior control system is not limited to the robot 100, and the behavior control system can be applied to various types of electronic equipment. Further, functions of a server 300 may be implemented by one or more computers. At least some functions of the server 300 may be implemented by a virtual machine. Further, at least some functions of the server 300 may be implemented on a cloud.

### Fifth Embodiment

A fifth embodiment is an example in which the response processing and the autonomous processing in the behavior control system of the second embodiment and the agent function of the fourth embodiment can be applied to the stuffed toy of the third embodiment. Hereinafter, portions having similar configurations to those of the first to fourth embodiments are denoted by the same reference numerals, and a description thereof is omitted.

A robot 100 (corresponding to a smartphone 50 housed in a stuffed toy 100N in the present embodiment) of the present embodiment performs the following processing.

The robot 100 generates a question according to attribute information of a user 10 who is a consultee and a concern of the user 10, and has a conversation with the user 10. Examples of the attribute information include an age, a gender, an occupation, family members, a medical history, and a lifestyle of the user 10. The robot 100 analyzes a content of an answer from the user 10 for the question, and a facial expression, an emotion, and a motion of the user 10, and determines whether a mental condition of the user 10 is favorable or poor. In determining whether the mental condition is favorable or poor, for example, condition levels classified into “healthy”, “preliminary stage of poor condition”, “early stage of poor condition”, “poor condition”, “treatment required”, and the like are determined.

In a case where it is determined that the condition level of the mental condition of the user 10 is any level other than “healthy”, the robot 100 proposes a cause of the poor condition and an improvement measure. Furthermore, in a case where it is determined that the condition level of the mental condition of the user 10 is “treatment required”, the robot 100 supports improvement of mental health of the user 10 in cooperation with a related institution. As the support for improvement of the mental health of the user 10, the robot 100 inputs the content of the answer from the user 10 for the question and an emotion value of the user 10 to a sentence generation model, and provides, to the user 10, a solution or advice for a concern of the user 10, the solution or advice being output from the sentence generation model.

After performing the above support, the robot 10 inquires of the user 10 about a mental health improvement status and determines whether or not the support performed by the robot 100 is appropriate based on an inquiry result and the emotion value of the user 10 at that time. The robot 10 trains the sentence generation model by feeding back the answer content from the user 10 for each conversation step and the emotion of the user 10 to the sentence generation model, and implements a conversation that maximizes a rate of resolving concerns. In the case of a partner from which the robot 10 has received a consultation in the past, the robot 10 has a conversation in consideration of a history, and also takes into consideration a change in situation of the consultation partner.

The robot 100 (corresponding to the smartphone 50 housed in the stuffed toy 100N in the present embodiment) performs processing of the following steps 1 to 5 in the case of performing the support for improving the mental health of the user 10.

(Step S1) The robot 100 acquires the attribute information of the user 10 and the concern of the user 10 through a conversation with the user 10.

(Step S2) The robot 100 generates a question corresponding to the attribute information of the user 10 acquired in step S1 and the concern of the user 10 and has a conversation. Specifically, a behavior determination unit 2236 acquires a question text output from the sentence generation model by adding a fixed sentence such as “What is an effective question to identify the root cause of the concern of the user?” to a text indicating the attribute information and a content of the concern of the user 10, and inputting the text to the sentence generation model. The behavior determination unit 2236 determines, as a behavior of the robot 100, to make an utterance corresponding to the acquired question text. A behavior control unit 2250 controls a control target 252 and makes an utterance corresponding to the acquired question text for the user 10.

(Step S3) The behavior determination unit 2236 analyzes a content of an answer from the user 10 for the question in step S2, and the facial expression, the emotion, and the motion of the user 10, and determines whether the mental condition of the user 10 is favorable or poor. In the analysis of the emotion of the user 10, the emotion determination unit 2232 determines the emotion value of the user 10 based on information analyzed by a sensor module unit 2210 and a state of the user 10 recognized by a user state recognition unit 2230. In determining whether the mental condition is favorable or poor, the robot 100 determines the condition levels classified into “healthy”, “preliminary stage of poor condition”, “early stage of poor condition”, “poor condition”, “treatment required”, and the like.

(Step S4) In a case where it is determined that the condition level of the mental condition of the user 10 is any level other than “healthy”, the robot 10 proposes a cause of the poor condition and an improvement measure. Specifically, the behavior determination unit 2236 acquires a solution or advice for the concern of the user 10 output from the sentence generation model by adding a fixed sentence “What is the solution to the concern of the user in this case?” to a text indicating the content of the answer from the user 10 for the question and the emotion value of the user 10 and inputting the text to the sentence generation model. The behavior determination unit 2250 determines, as the behavior of the robot 100, to make an utterance corresponding to the acquired solution or advice. The behavior control unit 2250 controls the control target 252 and makes an utterance corresponding to the acquired solution or advice for the user 10.

(Step S5) The robot 10 inquires of the user 10 about the mental health improvement status and determines whether or not the support performed by the robot 100 is appropriate in step S4 based on an inquiry result and the emotion value of the user 10 at that time. Specifically, the emotion determination unit 2232 determines the emotion value indicating the emotion of the user 10 based on the information analyzed by the sensor module unit 2210 and the state of the user 10 recognized by the user state recognition unit 2230. The robot 100 derives a probability that the support performed in step S4 is effective based on the emotion value of the user 10 and the inquiry result. The robot 10 trains the sentence generation model by feeding back the answer content from the user 10 for each conversation step and the emotion of the user 10 to the sentence generation model, and implements a conversation that maximizes a rate of resolving concerns. It is possible to use the probability that the support is effective as the rate of resolving the concern.

In this manner, the robot 100 can perform processing of responding to a concern consultation from the user.

As in the second embodiment, the behavior of the robot 100 may be determined using an emotion table (see Table 4) described above. For example, in a case where a behavior of the user is a behavior of saying “I have something I'd like to discuss”, the emotion of the robot 100 corresponds to an index number “2”, and the emotion of the user 10 corresponds to an index number “3”, a text “The robot is in a very pleasant state. The user is in a normally pleasant state. The user said, “I have something I'd like to discuss”. How should the robot respond?” is input to the sentence generation model to thereby acquire the behavior content of the robot. The behavior determination unit 2236 determines the behavior of the robot based on the behavior content.

The processing described in the fifth embodiment may be performed in each of the response processing and the autonomous processing in the behavior control system of the second embodiment, or may be performed in the agent function of the fourth embodiment.

### Sixth Embodiment

A sixth embodiment is an example in which the response processing and the autonomous processing in the behavior control system of the second embodiment and the agent function of the fourth embodiment can be applied to the stuffed toy of the third embodiment. Hereinafter, portions having similar configurations to those of the first to fifth embodiments are denoted by the same reference numerals, and a description thereof is omitted.

A robot 100 (corresponding to a smartphone 50 housed in a stuffed toy 100N in the present embodiment) of the present embodiment supports health management of a user 10. For example, the robot 100 assists dieting of the user 10 as an exclusive trainer by managing a meal and exercise while taking into account a physical condition of the user 10.

### (Meal Management)

The robot 100 acquires data regarding a meal ingested by the user 10 from a conversation with the user 10. At this time, the data regarding the meal is transmitted to and stored in a server 300, another external server, or the like. Examples of the “data regarding the meal” include a type, an amount, an intake time, and a calorie intake of food or drinks. The robot 100 is configured to acquire the data regarding the meal of the user 10 in a predetermined period from the server 300 so as to be able to grasp a change in a meal content of the user 10 and a preference of the user 10 for the meal.

The acquisition of the data regarding the meal is not limited to acquisition through a conversation between the user 10 and the stuffed toy 100N, and the data regarding the meal may be acquired using other methods. For example, the data regarding the meal may be acquired from a content input by the user 10 to a health management application, a meal content posted on a social network service (SNS) by the user 10, and a meal content appearing in an image acquired from a 2D camera 2203 of the stuffed toy 100N, a camera provided in a living room where the user 10 eats meals, or the like, and may be automatically stored in the server 300.

For example, in a case where the user 10 reports the meal content to the stuffed toy 100N, the robot 100 estimates calories of the ingested meal and notifies the user 10 of the calorie intake. Specifically, a behavior control unit 2250 controls a speaker 60 or a monitor of the stuffed toy 100N, which is a control target 2252, to notify the user 10 of the calorie intake. At this time, the robot 100 makes an utterance according to the emotion of the user 10. For example, in a case where the user 10 reports happily, the robot 100 may make a facial expression and an utterance showing empathy, such as “That looks delicious”, and in a case where the user 10 reports sadly, for example, “I ate too much”, the robot 100 may make an encouraging facial expression and utterance. Furthermore, for example, in a case where the user 10 has been overeating on consecutive days, the robot 100 may express a feeling of concern, for example, by saying “It seems you've been eating too much lately. Are you okay?”, provide advice to reduce food intake, and provide suggestions for healthy recipes. At this time, a recipe matched to the preference of the user 10 may be proposed. The robot 100 may also read the physical condition of the user 10 from greetings or the like, and in a case where the physical condition of the user 10 appears to be poor, the robot 100 may propose a recipe matched to the physical condition, such as a meal that is easy to digest. Furthermore, for example, the robot 100 may manage an alcohol intake of the user 10. For example, in the case of the user 10 who has set a weekly alcohol-free day, the robot 100 may make an utterance such as “Today is your alcohol-free day” on that day of the week.

### (Movement Management)

Furthermore, the robot 100 acquires data regarding the exercise performed by the user 10 from a conversation with the user 10. At this time, the data regarding the exercise is transmitted to and stored in the server 300, another external server, or the like. For example, in a case where a content of the exercise performed by the user 10 is conveyed to the stuffed toy 100N, the data regarding the exercise is transmitted to and stored in the server 300. Examples of the “data regarding the exercise” here include a type, an amount, a time, and consumed calories of the exercise performed by the user 10. The robot 100 is configured to acquire the data regarding the exercise of the user 10 in a predetermined period from the server 300 so as to be able to grasp a continuation status of the exercise of the user 10 and the preference of the user 10 for the exercise.

The acquisition of the data regarding the exercise is not limited to acquisition through a conversation between the user 10 and the stuffed toy 100N, and the data regarding the exercise may be acquired using other methods. For example, the data regarding the exercise may be acquired from a wearable device worn by the user 10, the health management application input by the user 10, an exercise content posted on an SNS by the user 10, the 2D camera 2203 of the stuffed toy 100N, a camera provided in a living room where the user 10 exercises, and the like.

For example, in a case where the user 10 has answered “I have run 10 km” to a question “Did you exercise today?”, the robot 100 makes a positive utterance such as “Good job!” or “Nice effort!” and makes a positive gesture such as applause or thumbs-up. At this time, the consumed calories of the exercise are estimated and reported to the user 10 as an exercise achievement. Specifically, a behavior control unit 2250 controls the speaker 60 or the monitor of the stuffed toy 100N, which is the control target 2252, to notify the user 10 of the consumed calories.

Furthermore, the robot 100 may ask the user 10 a question about a target weight and a target body fat percentage, store an answer for the question in the server 300, and support the dieting such that the target weight and the target body fat percentage are achieved. Furthermore, the current body weight, body fat percentage, muscle mass, and the like may be inquired together with the meal and exercise contents, and more accurate support may be provided based on a difference between a target value and a current value. Furthermore, for example, these pieces of information may be acquired from a healthcare meter used by the user 10.

Furthermore, the robot 100 may support the dieting such that a body shape of the user 10 approaches a desired body shape. For example, the robot 100 may ask the user 10 about a preference regarding the body shape such as slim, standard, or muscular and store an answer of the user 10 in the server 300 as the target body shape, thereby supporting the dieting of the user 10 such that the corresponding body shape is achieved. As an example, in a case where the user 10 aims for the standard body shape, the robot 100 may set the target weight corresponding to a BMI of 22 and propose the target weight to the user 10. Further, for example, a body shape of a celebrity who is of the same gender as the user 10 and is preferred by the user 10, such as a model or an athlete, may be proposed to the user 10 as the target body shape.

For example, in the case of proposing an exercise to the user 10, the robot 100 may propose the exercise matched to the preference of the user 10. For example, the robot 100 may grasp a favorite exercise of the user 10, such as yoga or muscle training, from the exercise performed by the user 10 so far, a conversation with the exercise user 10, a moving image frequently viewed by the user 10, and the like, and propose the exercise matched to the preference of the user 10. At this time, a video or music matched to the preference may be displayed on the monitor to encourage the exercise of the user 10. Further, for example, the robot 100 may acquire a program of a fitness center that the user 10 attends from the server 300 and propose participation in the program.

Furthermore, the robot 100 may read the physical condition of the user 10 from greetings or the like, and propose the exercise matched to the physical condition of the user 10. For example, in a case where the physical condition of the user 10 appears to be poor, the proposal of the exercise may be withheld. Furthermore, for example, in a case where the user 10 complains of a specific poor condition such as stiff shoulder or back pain, an exercise for solving the problem may be proposed. Furthermore, in a case where the physical condition of the user 10 appears to be favorable, intense exercise may be proposed.

As described above, the robot 100 can support the dieting of the user 10 as an exclusive trainer by proposing an appropriate meal and exercise together with an appropriate response such as a compliment or a concern based on information such as the calorie intake, the consumed calories, the continuation statuses of the meal and the exercise, and the physical condition of the user 10. The robot 100 may provide support for achieving the standard body type for the user 10 who is, for example, underweight, in addition to support for dieting.

### (Sleep Management)

Furthermore, the robot 100 may acquire data regarding sleep of the user 10 from the server 300, another external server, or the like. Examples of the “data regarding the sleep” here include a bedtime, a wake-up time, a sleep time, and sleep quality of the user 10.

For example, the data regarding the sleep is acquired from a conversation with the user 10 and transmitted to and stored in the server 300. As an example, in a case where the user 10 says “Good night” to the stuffed toy 100N, a time at which the user 10 says “Good night” is transmitted to and stored in the server 300 as the bedtime, and in a case where the user 10 says “Good morning” to the stuffed toy 100N, a time at which the user 10 says “Good morning” is transmitted to and stored in the server 300 as the wake-up time.

Furthermore, for example, the robot 100 may evaluate a sleep quality level of sleep in multiple stages based on a conversation with the user 10 and transmit the sleep quality level to the server 300 together with the above times. As an example, in response to a question from the stuffed toy 100N such as “Did you sleep well?”, the robot 100 evaluates the sleep quality level as Level 1 in a case where the user 10 has answered “I didn't sleep very well”, evaluates the sleep quality level as Level 2 in a case where the user 10 has answered “It was average”, evaluates the sleep quality level as Level 3 in a case where the user 10 has answered “I slept soundly”, and transmits the evaluated sleep quality level to the server 300.

The acquisition of the data regarding the sleep is not limited to acquisition through a conversation between the user 10 and the stuffed toy 100N, and the data regarding the sleep may be acquired using other methods. For example, the data regarding the sleep may be acquired from a wearable device worn by the user 10, the health management application input by the user 10, a sleep-related content posted on an SNS by the user 10, the 2D camera 2203 of the stuffed toy 100N, a camera provided in a bedroom where the user 10 sleeps, and the like.

The robot 100 assists the user 10 in managing sleep based on a sleep content and change of the user 10 and the emotion of the user 10 or an emotion of the robot 100. For example, the user 10 may be notified of a scheduled bedtime and a scheduled wake-up time set by the user 10. Furthermore, an utterance for urging the user to go to bed early may be made together with a facial expression of concern for the user 10 who has been experiencing continued lack of sleep. At this time, food, drinks, stretches, and the like that promote falling asleep easily may be proposed.

Furthermore, for example, in a case where the user 10 makes an utterance “I'm cold”, the robot 100 may propose food, drinks, and exercise that warm the body, may propose bathing, or may control air conditioning. Furthermore, for example, in a case where anger and excitement are read from the user 10, drinks, music, bathing, sleep, or the like that encourages deep breathing or relaxing may be proposed.

Furthermore, for example, the robot 100 may support health management according to a menstrual cycle for the female user 10. For example, a high-intensity exercise may be proposed during a follicular phase, iron-rich meals may be proposed during a menstruation phase, or stretches or meals for relaxing may be proposed during a luteal phase.

In this manner, the robot 100 can perform processing of comprehensively supporting the health management of the user 10 according to the preference of the user 10, a situation of the user 10, and a reaction of the user 10.

As in the second embodiment, the behavior of the robot 100 may be determined using an emotion table (see Table 4) described above. For example, in a case where a behavior of the user is a behavior of saying “I ran here”, the emotion of the robot 100 corresponds to an index number “2”, and the emotion of the user 10 corresponds to an index number “3”, a text “The robot is in a very pleasant state. The user is in a normally pleasant state. The user said, “I ran here”. How should the robot respond?” is input to the sentence generation model to thereby acquire the behavior content of the robot. The behavior determination unit 2236 determines the behavior of the robot based on the behavior content.

The processing described in the sixth embodiment may be performed in each of the response processing and the autonomous processing in the behavior control system of the second embodiment, or may be performed in the agent function of the fourth embodiment.

### Seventh Embodiment

In a seventh embodiment, the above-described agent system is applied to smart glasses. Portions having similar configurations to those of the first to sixth embodiments are denoted by the same reference numerals, and a description thereof is omitted.

FIG. 9H is a functional block diagram of an agent system 2700 implemented using some or all of functions of a behavior control system.

As shown in FIG. 9I, smart glasses 2720 are a glasses-type smart devices and are worn by a user 10 similarly to regular glasses. The smart glasses 2720 are an example of electronic equipment and a wearable terminal.

The smart glasses 2720 include the agent system 2700. A display included in a control target 2252B displays various types of information for the user 10. The display is, for example, a liquid crystal display. The display is provided, for example, at a lens portion of the smart glasses 2720, and a display content can be visually recognized by the user 10. A speaker included in the control target 2252B outputs a speech representing various types of information to the user 10.

The smart glasses 2720 include a touch panel (not shown), and the touch panel receives an input from the user 10.

An acceleration sensor 2206, a temperature sensor 2207, and a heart rate sensor 2208 of a sensor unit 2200B detect a state of the user 10. The sensors are merely examples, and it is a matter of course that other sensors may be mounted in order to detect the state of the user 10.

A microphone 2201 acquires a speech uttered by the user 10 or an environmental sound around the smart glasses 2720. A 2D camera 2203 can image the surroundings of the smart glasses 2720. The 2D camera 2203 is, for example, a CCD camera.

A sensor module unit 2210B includes a speech emotion recognition unit 2211 and an utterance understanding unit 2212. A communication processing unit 2280 of a control unit 2228B controls communication between the smart glasses 2720 and the outside.

FIG. 9I is a diagram showing an example of a usage aspect of the agent system 2700 in the smart glasses 2720. The smart glasses 2720 implement provision of various services to the user 10 using the agent system 2700. For example, in a case where the smart glasses 2720 are operated by the user 10 (for example, the user 10 inputs a speech to the microphone or taps the touch panel with a finger), the smart glasses 2720 start to use the agent system 2700. Here, using the agent system 2700 includes an aspect in which the smart glasses 2720 include and use the agent system 2700, and further includes an aspect in which a part (for example, the sensor module unit 2210B, a storage unit 2220, and the control unit 2228B) of the agent system 2700 is provided outside the smart glasses 2720 (for example, a server), and the smart glasses 2720 communicate with the outside to use the agent system 2700.

In a case where the user 10 operates the smart glasses 2720, a touchpoint is established between the agent system 2700 and the user 10. That is, service provision by the agent system 2700 is started. As described in the fourth embodiment, in the agent system 2700, a character (for example, a character of Audrey Hepburn) of an agent is set by a character setting unit 2276.

An emotion determination unit 2232 determines an emotion value indicating an emotion of the user 10 and an emotion value of the agent. Here, the emotion value indicating the emotion of the user 10 is estimated from various sensors included in the sensor unit 2200B mounted on the smart glasses 2720. For example, in a case where a heart rate of the user 10 detected by the heart rate sensor 2208 is elevated, the emotion value of “anxiety”, “fear”, or the like is estimated to be large.

Further, for example, in a case where a body temperature of the user exceeds an average body temperature as a result of measuring the body temperature using the temperature sensor 2207, the emotion value of “pain”, “suffering”, or the like is estimated to be large. Further, for example, in a case where it is detected by the acceleration sensor 2206 that the user 10 is performing any kind of sport, the emotion value of “pleasure” or the like is estimated to be large.

Further, for example, the emotion value of the user 10 may be estimated from a speech or utterance content of the user 10 acquired by the microphone 2201 mounted on the smart glasses 2720. For example, in a case where the user 10 is raising his/her voice, the emotion value of “anger” or the like is estimated to be large.

In a case where the emotion value estimated by the emotion determination unit 2232 is larger than a predetermined value, the agent system 2700 causes the smart glasses 2720 to acquire information regarding a surrounding situation. Specifically, for example, the 2D camera 2203 is caused to capture an image or a moving image indicating the surrounding situation (for example, a person or an object) of the user 10. Further, the microphone 2201 is caused to record ambient environmental sound. Examples of other information regarding the surrounding situation include a date, a time, location information, and information indicating weather. The information regarding the surrounding situation is stored in history data 2222 together with the emotion value. The history data 2222 may be implemented by an external cloud storage. As described above, the surrounding situation obtained by the smart glasses 2720 is stored in the history data 2222 as a so-called life log in a state of being associated with the emotion value of the user 10 at that time.

In the agent system 2700, information indicating the surrounding situation is stored in the history data 2222 in association with the emotion value. As a result, the agent system 2700 grasps personal information such as a hobby, a preference, or a personality of the user 10. For example, in a case where an image indicating a scene of watching baseball is associated with the emotion value of “happy” or “pleasure”, the agent system 2700 grasps the fact that the hobby of the user 10 is watching baseball and grasps a favorite team or player of the user 10 from the information stored in the history data 2222.

Then, in the case of having a dialogue with the user 10 or performing a behavior for the user 10, the agent system 2700 determines a dialogue content or a behavior content in consideration of a content of the surrounding situation stored in the history data 2222. It is a matter of course that the dialogue content or the behavior content may be determined in consideration of a dialogue history stored in the history data 2222 as described above in addition to the surrounding situation.

As described above, a behavior determination unit 2236 generates an utterance content based on a sentence generated by a sentence generation model. Specifically, the behavior determination unit 2236 generates the utterance content of the agent by inputting, to the sentence generation model, a text or speech input by the user 10, the emotions of both the user 10 and the agent determined by the emotion determination unit 2232, the conversation history stored in the history data 2222, a personality of the agent, and the like. Further, the behavior determination unit 2236 generates the utterance content of the agent by inputting the surrounding situation stored in the history data 2222 to the sentence generation model.

The generated utterance content is output by voice from the speaker mounted on the smart glasses 2720 to the user 10, for example. In this case, a synthesized voice corresponding to the character of the agent is used as the voice. A behavior control unit 2250 generates the synthesized voice by reproducing a voice style of the character (for example, Audrey Hepburn) of the agent, and generates the synthesized voice corresponding to the emotion of the character (for example, a voice with a forcible tone in a case where the emotion is “anger”). Further, the utterance content may be displayed on the display instead of or together with the voice output.

An RPA 2274 performs an operation according to a command (for example, a command of the agent acquired from a speech or text uttered by the user 10 through a dialogue with the user 10). For example, the RPA 2274 performs a behavior related to use of a service provider, such as information search, restaurant reservation, ticket arrangement, purchase of products or services, payment, route guidance, or translation.

Further, as another example, the RPA 2274 performs an operation of transmitting a content input by voice from the user 10 (for example, a child) through a dialogue with the agent to a counterpart (for example, parents). Examples of transmission means include message application software, chat application software, and mail application software.

In a case where the operation is performed by the RPA 2274, for example, a speech indicating that the operation is finished is output from the speaker mounted on the smart glasses 2720. For example, a speech such as “The reservation of the restaurant is completed” is output to the user 10. Further, for example, in a case where the restaurant is fully booked, a speech such as “The reservation could not be made. What would you like to do?” is output to the user 10.

As described above, the smart glasses 2720 use the agent system 2700 to provide various services to the user 10. In addition, since the smart glasses 2720 are worn by the user 10, the agent system 2700 can be used in various scenes such as at home, at work, and at a place outside the house.

In addition, since the smart glasses 2720 are worn by the user 10, the smart glasses 2720 are suitable for collecting the so-called life log of the user 10. Specifically, the emotion value of the user 10 is estimated based on detection results of various sensors or the like mounted on the smart glasses 2720 or recording results of the 2D camera 2203 or the like. Therefore, the emotion value of the user 10 can be collected in various scenes, and the agent system 2700 can provide a service or utterance content appropriate for the emotion of the user 10.

Further, in the smart glasses 2720, the surrounding situation of the user 10 can be obtained by the 2D camera 2203, the microphone 2201, and the like. Then, the surrounding situation and the emotion value of the user 10 are associated with each other. As a result, it is possible to estimate what kind of emotion the user 10 has in what kind of situation. As a result, accuracy in a case where the agent system 2700 grasps the hobby and the preference of the user 10 can be improved. Then, as the agent system 2700 accurately grasps the hobby and the preference of the user 10, the agent system 2700 can provide a service or an utterance content appropriate for the hobby and the preference of the user 10.

Further, the agent system 2700 can also be applied to other wearable terminals (electronic equipment that can be worn on the body of the user 10, such as a pendant, a smart watch, an earring, a bracelet, or a hairband). In a case where the agent system 2700 is applied to a smart pendant, a speaker serving as the control target 2252B outputs a speech representing various types of information to the user 10. The speaker is, for example, a speaker capable of outputting a sound having directionality. The speaker is set to have directionality toward the ear of the user 10. As a result, the sound is suppressed from reaching a person other than the user 10. The microphone 2201 acquires a speech uttered by the user 10 or an environmental sound around the smart pendant. The smart pendant is worn so as to be suspended from the neck of the user 10. Therefore, the smart pendant is positioned relatively close to the mouth of the user 10 while being worn. As a result, acquisition of a speech uttered by user 10 is facilitated.

In the above embodiment, a case where the robot 100 recognizes the user 10 by using a face image of the user 10 has been described, but the disclosed technology is not limited to such an aspect. For example, the robot 100 may recognize the user 10 by using a voice uttered by the user 10, a mail address of the user 10, an ID of a social network service (SNS) of the user 10, an ID card in which a wireless IC tag is embedded and which is possessed by the user 10, or the like.

The robot 100 is an example of electronic equipment including the behavior control system. An application target of the behavior control system is not limited to the robot 100, and the behavior control system can be applied to various types of electronic equipment. Further, functions of a server 300 may be implemented by one or more computers. At least some functions of the server 300 may be implemented by a virtual machine. Further, at least some functions of the server 300 may be implemented on a cloud.

FIG. 4 schematically shows an example of a hardware configuration of a computer 1200 that functions as the smartphone 50, the robot 100, the server 300, and the agent systems 2500 and 2700.

### Eighth Embodiment

A robot 100 further includes a specific processing unit 290 in the configurations of the first to sixth embodiments.

Processing performed by the specific processing unit 290 in a case where the robot 100 performs processing of creating pitching information regarding a next pitch to be thrown by a specific pitcher as the specific processing will be described.

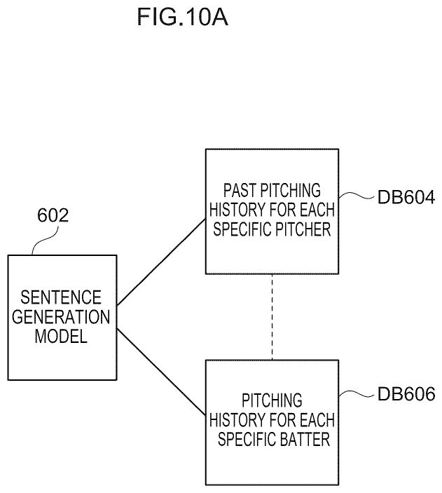

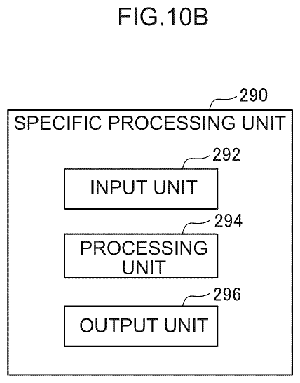

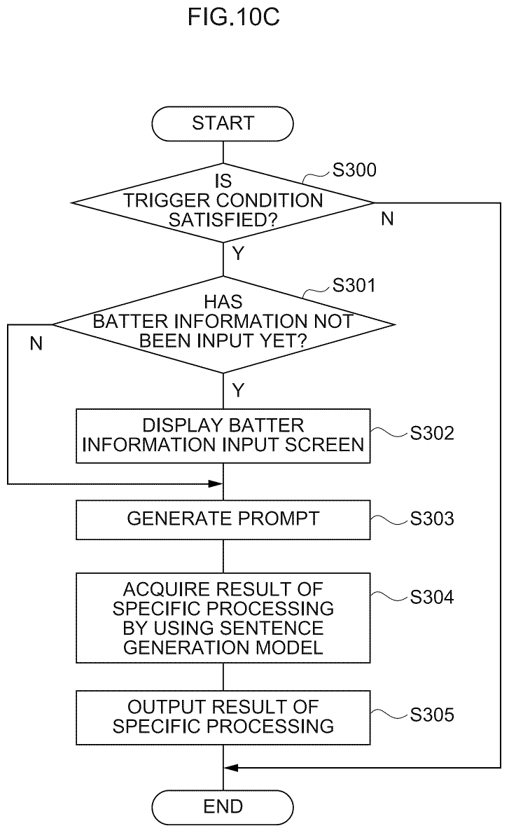

In the specific processing in the present embodiment, as shown in FIG. 10A, a sentence generation model 602 used to create the pitching information is connected to a past pitching history DB 604 for each specific pitcher and a past pitching history DB 606 for each specific batter. The past pitching history DB 604 for each specific pitcher stores a past pitching history associated with each registered specific pitcher. Specific examples of a content stored in the past pitching history DB 604 for each specific pitcher include a pitching date, the number of pitches, a pitch type, a pitching course, an opposing batter, and a result (such as hit, strikeout, or homerun). The past pitching history DB 606 for each specific batter stores a past pitching history associated with each registered specific batter. Specific examples of a content stored in the past pitching history DB 606 for each specific batter include a pitching date, the number of pitches, a pitch type, a pitching course, an opposing batter, and a result (such as hit, strikeout, or homerun). The specific sentence generation model 602 is subjected to fine tuning in advance to additionally learn each piece of information stored in the DBs 604 and 606.

As shown in FIG. 10B, the specific processing unit 290 includes an input unit 292, a processing unit 294, and an output unit 296.

The input unit 292 receives a user input. Specifically, a speech input from a user, a text input via a mobile terminal, or the like is acquired. For example, the user inputs a text or speech requesting the pitching information regarding the next pitch to be thrown by the specific pitcher, such as “Give me information regarding the next pitch to be thrown by the specific pitcher XX YY”.

The processing unit 294 determines whether or not a predetermined trigger condition is satisfied. For example, the trigger condition is reception of the text or speech requesting the pitching information regarding the next pitch to be thrown by the specific pitcher, such as “Give me information regarding the next pitch to be thrown by the specific pitcher XX YY”.

The processing unit 294 may optionally cause the user to input opposing batter information in a case where the trigger condition is satisfied. The batter information may be a specific batter (batter name) or may be simply a distinction between a left-handed batter and a right-handed batter.

Then, the processing unit 294 inputs a text indicating an instruction for obtaining data for the specific processing to the sentence generation model, and acquires a processing result based on an output of the sentence generation model. More specifically, as the specific processing, the processing unit 294 performs processing of generating a sentence (prompt) for instructing creation of the pitching information regarding the next pitch to be thrown by the specific pitcher, the pitching information being received by the input unit 292, and inputting the generated sentence to the sentence generation model 602, and acquires the pitching information regarding the next pitch to be thrown by the specific pitcher. For example, the processing unit 294 generates a prompt such as “Specific pitcher XX YY, count of 2 balls, 1 strike, and 2 outs, opposing batter YY XX, please create pitching information regarding the next pitch”. The pitching information includes the pitch type and the pitch course (distinguishing between outside, inside, high, and low). Then, the processing unit 294 acquires, for example, an answer such as “Specific pitcher XX YY, the next pitch is likely to be outside, low, and a fastball” from the sentence generation model 602.

The processing unit 294 may perform the specific processing using a state of the user or a state of the robot 100 and the sentence generation model. Furthermore, the processing unit 294 may perform the specific processing using an emotion of the user or an emotion of the robot 100 and the sentence generation model.

The output unit 296 controls a behavior of the robot 100 so as to output a result of the specific processing. Specifically, the pitching information regarding the next pitch to be thrown by the specific pitcher is displayed on a display device provided in the robot 100 or is uttered by the robot 100, or these pieces of information are transmitted as a message to a user of a message application of the mobile terminal of the user.

A part of the robot 100 (for example, a sensor module unit 210, a storage unit 220, and a control unit 228) may be provided outside the robot 100 (for example, a server), and the robot 100 may function as each unit of the robot 100 by communicating with the outside.

FIG. 10C schematically shows an example of an operation flow related to an operation in which the robot 100 performs the specific processing of creating the pitching information regarding the next pitch to be thrown by the specific pitcher. The operation flow shown in FIG. 4C is repeatedly and automatically performed, for example, every lapse of a certain period of time.

In step S300, the processing unit 294 determines whether or not the predetermined trigger condition is satisfied. For example, the processing unit 294 determines whether or not information indicating a request for creation of the pitching information regarding the next pitch to be thrown by the specific pitcher, such as “Give me information regarding the next pitch to be thrown by the specific pitcher XX YY” has been input from the user 10. In a case where the trigger condition is satisfied, the processing proceeds to step S301. On the other hand, in a case where the trigger condition is not satisfied, the specific processing ends.

In step S301, the processing unit 294 determines whether or not the opposing batter information has been input from the user, and in a case where the opposing batter information has not been input, the processing unit 294 displays an input screen for causing the user to input the information on the display device provided in the robot 100 in step S302, and requests the user to input the opposing batter information. In a case where the opposing batter information has been input from the user, the processing proceeds to step S303.

In a case where the batter information has been input by the user or there is no input for a predetermined time, the processing proceeds to step S303, and the processing unit 294 adds an instructional sentence for obtaining a result of the specific processing to a text representing the input and generates a prompt. For example, the processing unit 294 generates a prompt such as “Specific pitcher XX YY, count of 2 balls, 1 strike, and 2 outs, opposing batter YY XX, please create pitching information regarding the next pitch”.

In step S304, the processing unit 294 inputs the generated prompt to the sentence generation model 602, and acquires an output of the sentence generation model 602, that is, the pitching information regarding the next pitch to be thrown by the specific pitcher.

In step S305, the output unit 296 controls the behavior of the robot 100 so as to output the result of the specific processing, and ends the specific processing. As the output result of the specific processing, for example, a text such as “Specific pitcher XX YY, the next pitch is likely to be outside, low, and a fastball” is displayed.

Based on the pitching information, a batter facing the specific pitcher XX YY can predict the next pitch and prepare at the plate according to the pitching information.

Next, processing performed by the specific processing unit 290 in a case where the agent system 2500 of the fourth embodiment and the agent system 2700 of the seventh embodiment described above perform the specific processing using the sentence generation model 602 will be described.

In the specific processing in the agent system, the specific processing for the pitching information regarding the next pitch to be thrown by the specific pitcher is performed, and a behavior of the agent is controlled so as to output a result of the specific processing. At this time, an utterance content of the agent for having a conversation with the user 10 is determined as the behavior of the agent, and the utterance content of the agent is output by a speaker or a display serving as a control target 252B as at least one of a speech and a text.

The processing unit 294 may perform the specific processing using the state of the user or a state of the agent and the sentence generation model. Furthermore, the processing unit 294 may perform the specific processing using the emotion of the user or an emotion of the agent and the sentence generation model.

### (Supplementary Note 1)

A behavior control system including:
an emotion determination unit that determines an emotion of a user or an emotion of a robot; and
a behavior determination unit that generates a behavior content of the robot for a behavior of the user and the emotion of the user or the emotion of the robot based on a dialogue function of causing the user and the robot to have a dialogue with each other, and determines a behavior of the robot corresponding to the behavior content,
in which the behavior determination unit generates, as the behavior content, an utterance content for a consultation from the user based on information regarding a specific person.

### (Supplementary Note 2)

The behavior control system according to Supplementary Note 1, in which the behavior determination unit generates the utterance content for a content of the consultation from the user.

### (Supplementary Note 3)

The behavior control system according to Supplementary Note 2, in which the behavior determination unit reflects a voice or a speech habit of the specific person in the utterance content.

### (Supplementary Note 4)

The behavior control system according to Supplementary Note 3, in which the behavior determination unit determines a gesture of the robot corresponding to the utterance content.

### (Supplementary Note 5)

A behavior control system including:
an emotion determination unit that determines an emotion of a user or an emotion of a robot; and
a behavior determination unit that generates a behavior content of the robot for a behavior of the user and the emotion of the user or the emotion of the robot based on a dialogue function that causes the user and the robot to have a dialogue with each other, and determines a behavior of the robot corresponding to the behavior content,
in which in a case where it is determined that the user is a specific user including an individual who lives alone in isolation, the behavior determination unit switches to a specific mode in which the behavior of the robot is determined based on a communication count larger than a communication count in a normal mode in which the behavior is determined for a user other than the specific user.

### (Supplementary Note 6)

The behavior control system according to Supplementary Note 5, in which in a case where there is no dialogue with the specific user for a certain period of time in the specific mode, the behavior determination unit contacts a predetermined emergency contact.

### (Supplementary Note 7)

A behavior control system including:
an emotion determination unit that determines an emotion of a user or an emotion of a robot; and
a behavior determination unit that generates a behavior content of the robot for a behavior of the user and the emotion of the user or the emotion of the robot based on a dialogue function of causing the user and the robot to have a dialogue with each other, and determines a behavior of the robot corresponding to the behavior content, in which
the robot is installed in a meeting room, and
the behavior determination unit acquires a result of summarizing minutes of a past meeting held at the meeting room, and in a case where a statement whose content is similar to the summarized minutes has been made in a new meeting different from the past meeting, the behavior determination unit determines, as the behavior of the robot, outputting of advice information for the statement.

### (Supplementary Note 8)

A behavior control system including:
a user state recognition unit that recognizes a user state including a behavior of a user;
an emotion determination unit that determines an emotion of the user or an emotion of a robot; and
a behavior determination unit that determines a behavior of the robot corresponding to the user state and the emotion of the user or the emotion of the robot based on a sentence generation model having a dialogue function of causing the user and the robot to have a dialogue with each other,
in which the behavior determination unit generates a question corresponding to a concern of the user by using the sentence generation model, and determines, as the behavior of the robot, to make an utterance corresponding to the question.

### (Supplementary Note 9)

The behavior control system according to Supplementary Note 8, in which the behavior determination unit analyzes a content of an answer from the user for the question, and a facial expression, the emotion, and a motion of the user, and determines whether a mental condition of the user is favorable or poor.

### (Supplementary Note 10)

The behavior control system according to Supplementary Note 9, in which the behavior determination unit acquires a solution or advice for the concern of the user by using the sentence generation model according to a result of determining whether the mental condition of the user is favorable or poor, and determines, as the behavior of the robot, to make an utterance corresponding to the acquired solution or advice.

### (Supplementary Note 11)

The behavior control system according to Supplementary Note 8, in which the robot is mounted on a stuffed toy or is connected wirelessly or by wire to control target equipment mounted on a stuffed toy.

### (Supplementary Note 12)

The behavior control system according to Supplementary Note 11, in which
the control target equipment is a speaker, and
a microphone or a camera is mounted on the stuffed toy.

### (Supplementary Note 13)

The behavior control system according to Supplementary Note 12, in which the camera is attached to an eye included in a face of the stuffed toy, the microphone is attached to an ear, and the speaker is attached to a mouth.

### (Supplementary Note 14)

The behavior control system according to Supplementary Note 11, in which
a wireless power receiving unit that receives wireless power supply from an external wireless power transmitting unit is disposed inside the stuffed toy, and
the control target equipment or the robot receives power via the wireless power receiving unit.

### (Supplementary Note 15)

A behavior control system including:
a user state recognition unit that recognizes a user state including a behavior of a user;
an emotion determination unit that determines an emotion of the user or an emotion of electronic equipment; and
a behavior determination unit that determines a behavior of the electronic equipment corresponding to the user state and the emotion of the user or the emotion of the electronic equipment based on a sentence generation model having a dialogue function of causing the user and the electronic equipment to have a dialogue with each other,
in which the behavior determination unit determines the behavior of the electronic equipment that supports health management of the user.

### (Supplementary Note 16)

The behavior control system according to Supplementary Note 15, in which the electronic equipment is mounted on a stuffed toy or is connected wirelessly or by wire to control target equipment mounted on a stuffed toy.

### (Supplementary Note 17)

The behavior control system according to Supplementary Note 16, in which
the control target equipment is a speaker, and
a microphone or a camera is mounted on the stuffed toy.

### (Supplementary Note 18)

The behavior control system according to Supplementary Note 17, in which the camera is attached to an eye included in a face of the stuffed toy, the microphone is attached to an ear, and the speaker is attached to a mouth.

### (Supplementary Note 19)

The behavior control system according to Supplementary Note 16, in which
a wireless power receiving unit that receives wireless power supply from an external wireless power transmitting unit is disposed inside the stuffed toy, and
the control target equipment or the electronic equipment receives power via the wireless power receiving unit.

### (Supplementary Note 20)

The behavior control system according to any one of Supplementary Notes 15 to 19, in which the electronic equipment is a robot.

### (Supplementary Note 21)

A behavior control system including:
a state recognition unit that recognizes a user state including a behavior of a user and a state of electronic equipment;
an emotion determination unit that determines an emotion of the user or an emotion of the electronic equipment; and
a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing, in which
the equipment operation includes comforting the user, and
in a case where the behavior determination unit determines, as the behavior of the electronic equipment, to comfort the user, the behavior determination unit determines an utterance content corresponding to the user state and the emotion of the user.

### (Supplementary Note 22)

The behavior control system according to Supplementary Note 21, in which
the electronic equipment is a robot, and
the behavior determination unit determines, as a behavior of the robot, any one of a plurality of types of robot behaviors including doing nothing.

### (Supplementary Note 23)

The behavior control system according to Supplementary Note 22, in which
the behavior determination model is a sentence generation model having a dialogue function, and
the behavior determination unit inputs a text representing at least one of the user state, a state of the robot, the emotion of the user, and an emotion of the robot and a text for inquiry about the robot behavior to the sentence generation model, and determines the behavior of the robot based on an output of the sentence generation model.

### (Supplementary Note 24)

The behavior control system according to Supplementary Note 22 or 23, in which the robot is mounted on a stuffed toy or is connected wirelessly or by wire to control target equipment mounted on a stuffed toy.

### (Supplementary Note 25)

The behavior control system according to Supplementary Note 22 or 23, in which the robot is an agent for having a dialogue with the user.

### (Supplementary Note 26)

A behavior control system including:
a state recognition unit that recognizes a user state including a behavior of a user and a state of electronic equipment;
an emotion determination unit that determines an emotion of the user or an emotion of the electronic equipment;
a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing; and
a storage control unit that stores, in history data, event data including an emotion value determined by the emotion determination unit and data including the behavior of the user, in which
the equipment operation includes provision of advice on health to the user, and
in a case where the behavior determination unit determines, as the behavior of the electronic equipment, to provide the advice on health to the user, the behavior determination unit provides the advice on health to the user.

### (Supplementary Note 27)

The behavior control system according to Supplementary Note 26, in which
the electronic equipment is a robot, and
the behavior determination unit determines, as a behavior of the robot, any one of a plurality of types of robot behaviors including doing nothing.

### (Supplementary Note 28)

The behavior control system according to Supplementary Note 27, in which
the behavior determination model is a sentence generation model having a dialogue function, and
the behavior determination unit inputs a text representing at least one of the user state, a state of the robot, the emotion of the user, and an emotion of the robot and a text for inquiry about the robot behavior to the sentence generation model, and determines the behavior of the robot based on an output of the sentence generation model.

### (Supplementary Note 29)

The behavior control system according to Supplementary Note 27 or 28, in which the robot is mounted on a stuffed toy or is connected wirelessly or by wire to control target equipment mounted on a stuffed toy.

### (Supplementary Note 30)

The behavior control system according to Supplementary Note 27 or 28, in which the robot is an agent for having a dialogue with the user.

### (Supplementary Note 31)

A behavior control system including:
a state recognition unit that recognizes a user state including a behavior of a user and a state of electronic equipment;
an emotion determination unit that determines an emotion of the user or an emotion of the electronic equipment; and
a behavior determination unit that determines, as a behavior of the electronic equipment, any one of a plurality of types of equipment operations including performing no operation by using at least one of the user state, the state of the electronic equipment, the emotion of the user, and the emotion of the electronic equipment, and a behavior determination model at a predetermined timing, in which
the equipment operation includes provision of advice on a pregnant woman, and
in a case where the behavior determination unit determines, as the behavior of the electronic equipment, to provide the advice on a pregnant woman, the behavior determination unit collects information regarding at least one of a pregnancy period and a post-partum period, and provides the advice on a pregnant woman based on the collected information.

### (Supplementary Note 32)

The behavior control system according to Supplementary Note 31, in which
the electronic equipment is a robot, and
the behavior determination unit determines, as a behavior of the robot, any one of a plurality of types of robot behaviors including doing nothing.

### (Supplementary Note 33)

The behavior control system according to Supplementary Note 32, in which
the behavior determination model is a sentence generation model having a dialogue function, and
the behavior determination unit inputs a text representing at least one of the user state, a state of the robot, the emotion of the user, and an emotion of the robot and a text for inquiry about the robot behavior to the sentence generation model, and determines the behavior of the robot based on an output of the sentence generation model.

### (Supplementary Note 34)

The behavior control system according to Supplementary Note 32 or 33, in which the robot is mounted on a stuffed toy or is connected wirelessly or by wire to control target equipment mounted on a stuffed toy.

### (Supplementary Note 35)

The behavior control system according to claim 2 or 3, in which the robot is an agent for having a dialogue with the user.

### (Supplementary Note 36)

An information processing system including:
an input unit that receives a user input;
a processing unit that performs specific processing using a sentence generation model that generates a sentence corresponding to input data; and
an output unit that controls a behavior of electronic equipment so as to output a result of the specific processing,
in which in a case where pitching information regarding a next pitch to be thrown by a specific pitcher is requested, the processing unit performs, as the specific processing, processing of generating a sentence for instructing creation of the pitching information received by the input unit, and inputting the generated sentence to the sentence generation model, and causes the output unit to output the created pitching information as the result of the specific processing.

### (Supplementary Note 37)

The information processing system according to Supplementary Note 36, in which the pitching information includes pitch type information and pitch course information.

### (Supplementary Note 38)

The information processing system according to Supplementary Note 36, in which
the input unit receives an input of the specific pitcher from a user, and
the processing unit uses, as the sentence generation model, a model that has learned a past pitching history of the input specific pitcher.

### (Supplementary Note 39)

The information processing system according to claim 3, in which the processing unit uses, as the sentence generation model, a model trained based on a past pitching history and a result of the specific pitcher.

### (Supplementary Note 40)

The information processing system according to Supplementary Note 36, in which
the input unit receives an input of a specific batter from a user, and
the processing unit uses, as the sentence generation model, a model that has learned past pitching history information corresponding to the input specific batter.

### (Supplementary Note 41)

The information processing system according to Supplementary Note 40, in which the processing unit uses, as the sentence generation model, a model trained based on a past pitching history and a result associated with the specific batter.

### (Supplementary Note 42)

The information processing system according to Supplementary Note 36, in which the electronic equipment is an information communication terminal or a wearable terminal.

### (Supplementary Note 43)

The information processing system according to claim 7, in which the wearable terminal is a glasses-type terminal.

### (Supplementary Note 44)

The information processing system according to Supplementary Note 36, in which the electronic equipment is a robot.

### (Supplementary Note 45)

The information processing system according to Supplementary Note 44, in which the robot is mounted on a stuffed toy or is connected wirelessly or by wire to control target equipment mounted on a stuffed toy.

The disclosures of Japanese Patent Application No. 2023-065943, Japanese Patent Application No. 2023-065924, Japanese Patent Application No. 2023-064496, Japanese Patent Application No. 2023-072773, Japanese Patent Application No. 2023-073826, Japanese Patent Application No. 2023-120321, Japanese Patent Application No. 2023-075229, Japanese Patent Application No. 2023-081014 and Japanese Patent Application No. 2023-083456 are incorporated herein by reference in their entireties.

All documents, patent applications, and technical standards mentioned herein are incorporated herein by reference to the same extent as if each individual document, patent application, and technical standard were specifically and individually stated.

1. A behavior control system comprising:
a memory; and
at least one processor coupled to the memory,
the at least one processor being configured to:
determine an emotion of a user or an emotion of a robot; and
generate a behavior content of the robot for a behavior of the user and the emotion of the user or the emotion of the robot based on a dialogue function of causing the user and the robot to have a dialogue with each other, and determines a behavior of the robot corresponding to the behavior content,
wherein the at least one processor generates, as the behavior content, an utterance content for a consultation from the user based on information regarding a specific person.

2. The behavior control system according to claim 1, wherein the at least one processor generates the utterance content for a content of the consultation from the user.

3. The behavior control system according to claim 2, wherein the at least one processor reflects a voice or a speech habit of the specific person in the utterance content.

4. The behavior control system according to claim 3, wherein the at least one processor determines a gesture of the robot corresponding to the utterance content.

5. The behavior control system according to claim 1,
wherein the at least one processor is configured, when it is determined that the user is a specific user including an individual who lives alone in isolation, the behavior determination unit switches to a specific mode in which the behavior of the robot is determined based on a communication count larger than a communication count in a normal mode in which the behavior is determined for a user other than the specific user.

6. The behavior control system according to claim 1,
the robot is installed in a meeting room, and
wherein the at least one processor acquires a result of summarizing minutes of a past meeting held at the meeting room, and in a case where a statement whose content is similar to the summarized minutes has been made in a new meeting different from the past meeting, the behavior determination unit determines, as the behavior of the robot, outputting of advice information for the statement.

7. The behavior control system according to claim 1,
wherein the at least one processor generates a question corresponding to a concern of the user by using the sentence generation model, and determines, as the behavior of the robot, to make an utterance corresponding to the question.

8-11. (canceled)
